(19) United States
(12) Patent Application Publication
(10) Pub. No.: US 2009/0099187 A1
(43) Pub. Date: Apr. 16, 2009

(54) Novel 8-Sulfonyl-3 Aminosubstituted Chroman or Tetrahydronaphtalene Derivatives Modulating the 5HT6 Receptor

(75) Inventors: Gunnar Nordvall, Sodertalje (SE); Carl Petersson, Sodertalje (SE); Laszlo Rakos, Sodertalje (SE); Daniel Sohn, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 11/914,566

(22) PCT Filed: May 22, 2006
(86) PCT No.: PCT/SE06/00592

§ 371 Date: Aug. 26, 2008

(30) Foreign Application Priority Data
May 23, 2005 (SE) 0501165-5
May 23, 2005 (SE) 0501167-1

(51) Int. Cl. A61K 31/5375; C07C 311/21; A61K 31/18; C07D 207/09; C07D 311/58; A61K 31/352; A61K 31/402; C07D 265/30

(52) U.S. Cl. 514/239.5; 564/86; 514/603; 544/159; 548/578; 514/429; 514/456; 549/404

(57) ABSTRACT

The present invention relates to new compounds of formula (I), wherein R¹ to R¹², P, X, Q and n are as defined as in formula I, or salts, solvates or solvated salts thereof, processes for their preparation and to new intermediates used in the preparation thereof, pharmaceutical formulations containing said compounds and to the use of said compounds in therapy.

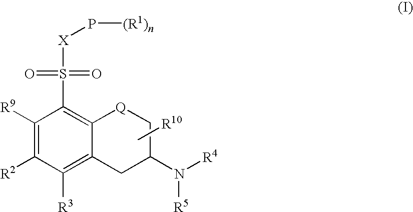

### FIELD OF THE INVENTION

The present invention relates to new compounds, to pharmaceutical formulations containing said compounds and to the use of said compounds in therapy. The present invention further relates to processes for the preparation of said compounds and to intermediates use in the preparation thereof.

### BACKGROUND OF THE INVENTION

Serotonin (5-hydroxy-tryptamine) (5-HT) receptors play an important role in many physiological and pathological functions like anxiety, sleep regulation, aggression, feeding and depression. The 5-HT receptors are distributed throughout the body and can be divided into seven different 5-HT receptor subtypes, i.e. 5-HT1-5-HT7, with different properties. The 5-HT6 receptor is mostly found in the central nervous system (CNS). From in situ hybridization studies it is known that the 5-HT6 receptor in rat brain is localized in areas like striatum, nucleus accumbens, olfactory tubercle and hippocampal formation (Ward et al., Neuroscience, 64, p 1105-1111, 1995).

Scientific research has revealed a potential therapeutic use for modulators of the 5-HT6 receptor, especially with regard to various CNS disorders. Blocking 5-HT6 receptor function has been shown to enhance cholinergic transmission (Bentley et al, Br J Pharmacol 126: 1537-1542, 1999; Riemer et al J Med Chem 46, 1273-1276). 5-HT6 antagonist have also been shown to reverse cognitive deficits in in vivo cognition models induced by the muscarinic antagonist scopolamine (Woolley et al. Psychopharmacology, 170, 358-367, 2003; Foley et al. Neuropsychopharmacology, 29 93-100, 2004)

Studies have shown that 5-HT6 antagonists increase levels of glutamate and aspartate in the frontal cortex and dorsal hippocampus as well as acetylcholine in the frontal cortex. These neurochemicals are known to be involved in memory and cognition (Dawson et al., Neuropsychopharmacology, 25(5), p 662-668, 2001) (Gerard et al., Brain Res., 746, p 207-219, 1997) (Riemer et al J Med Chem 46(7), p 1273-1276, 2003).

Acetylcholinesterase inhibitors increase the levels of acetylcholine in the CNS and are used in the treatment of cognitive disorders such as Alzheimer's disease. 5-HT6 antagonists may therefore be used in the treatment of cognitive disorders.

Studies have also shown that 5-HT6 antagonist increases the level of dopamine and noradrenaline in the medial prefrontal cortex (Lacroix et al. Synapse 51, 158-164, 2004). In addition, 5-HT₆ receptor antagonists have been shown to improve performance in the attentional set shifting task (Hatcher et al. Psychopharmacology 181(2):253-9, 2005). Therefore, 5-HT6 ligands are expected to be useful in the treatment of disorders where cognitive deficits are a feature, such as schizophrenia. Several antidepressants and atypical antipsychotics bind to the 5-HT6 receptor and this may be a factor in their profile of activities (Roth et al., J. Pharm. Exp. Therapeut., 268, 1402-1420, 1994; Sleight et al., Exp. Opin. Ther. Patents, 8, 1217-1224, 1998; Kohen et al., J. Neurochem., 66(1), p 47-56, 1996; Sleight et al. Brit. J. Pharmacol., 124, p 556-562, 1998; Bourson et al., Brit. J. Pharmacol., 125, p 1562-1566, 1998).

Stean et al., (Brit. J. Pharmacol. 127 Proc. Supplement 131P, 1999) have described the potential use of 5-HT6 modulators in the treatment of epilepsy. 5-HT6 receptors have also been linked to generalized stress and anxiety states (Yoshioka et al., Life Sciences, 62, 17/18, p 1473-1477, 1998). 5-HT6 agonists have been shown to elevate levels of GABA in brain regions associated with anxiety and shown positive effects in models predictive of obsessive-compulsive disorder (Schechter et al. NeuroRx. 2005 October; 2(4): 590-611). The use of modulators for this receptor is therefore expected for a wide range of CNS disorders.

Pullagurla et al (Pharmacol Biochem Behav. 78(2):263-8, 2004) have described the potential use of 5-HT6 antagonists in disorders were the dopamine transmission is affected, for example a combination between a 5-HT6 antagonist and a dopamine enhancer for example levodopa/carbidopa or amantadine would be expected to have a advantages compared to a dopamine enhancer alone.

Moreover, a reduction in food intake in rats has been reported using 5-HT6 receptor modulators (Bentley et al., Br. J. Pharmacol. Suppl. 126, P66, 1999; Bentley et al. J. Psychopharmacol. Supl. A64, 255, 1997; Pendharkar et al Society for Neuroscience, 2005). 5-HT6 receptor modulators may therefore also be useful in the treatment of feeding disorders like anorexia, obesity, bulimia and similar disorders and also type 2 diabetes.

The object of the present invention is to provide compounds exhibiting a modulating activity at the 5-hydroxy-tryptamine 6 receptor (5HT6). The compounds of the present invention have excellent selectivity and activity for the 5HT6 receptor.

### DETAILED DESCRIPTION OF THE INVENTION

The object of the present invention is to provide compounds exhibiting a modulating activity at the 5HT6 receptor.

The present invention provides compounds of formula I

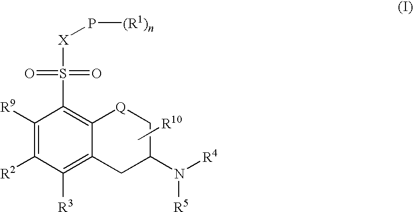

wherein:
P is C₆₋₁₀arylC₀₋₆alkyl, C₅₋₁₁heteroarylC₀₋₆alkyl, C₃₋₇cycloalkylC₀₋₆alkyl, C₃₋₇heterocycloalkylC₀₋₆alkyl or C₁₋₁₀alkyl;
R¹ is hydrogen, hydroxy, halogen, C₁₋₁₀alkyl, C₂₋₁₀alkenyl, C₂₋₁₀alkynyl, C₁₋₁₀alkoxy, N(R¹¹)₂, C₆₋₁₀arylC₀₋₆alkyl, C₅₋₆heteroarylC₀₋₆alkyl, C₁₋₆haloalkyl, C₁₋₆haloalkylO, R⁷OC₀₋₆alkyl, cyano, SR⁷, R⁷SO₂C₀₋₆alkyl, SOR⁷, R⁷CON(R⁸)C₀₋₆alkyl, NR⁸SO₂R⁷, COR⁷, COOR⁷, OSO₂R⁷, (R⁸)₂NCOC₀₋₆alkyl, SO₂N(R⁸)₂, N(R⁸)CON(R⁸)₂, NO₂ or oxo;
n is 0, 1, 2, 3 or 4;
X is a single bond, O, C₁₋₃alkyl or NR⁶, or X is N in a C₅₋₁₂heteroaryl;

### Q is CH or O;

R² is hydrogen, hydroxy, halogen, C₁₋₁₀alkyl, C₂₋₁₀alkenyl, C₂₋₁₀alkynyl, C₁₋₁₀alkoxy, N(R¹¹)₂, C₆₋₁₀arylC₀₋₆alkyl, C₅₋₆heteroarylC₀₋₆alkyl, C₁₋₆haloalkyl, C₁₋₆haloalkylO, R⁷OC₀₋₆alkyl, cyano, SR⁷, SO₂R⁸, SOR⁷, N(R⁸)COR⁷, N(R⁸)SO₂R⁷, COR⁷, COOR⁷, OSO₂R⁷, CON(R⁸)₂ or SO₂N(R⁸)₂;
R³ is hydrogen, hydroxy, halogen, C₁₋₁₀alkyl, C₂₋₁₀alkenyl, C₂₋₁₀alkynyl, C₁₋₁₀alkoxy, N(R¹¹)₂, C₆₋₁₀arylC₀₋₆alkyl, C₅₋₆heteroarylC₀₋₆alkyl, C₁₋₆haloalkyl, C₁₋₆haloalkylO, R⁷OC₀₋₆alkyl, cyano, SR⁷, SO₂R⁷, SOR⁷, N(R⁸)COR⁷, N(R⁸)SO₂R⁷, COR⁷, COOR⁷, OSO₂R⁷, CON(R⁸)₂ or SO₂N(R⁸)₂;
R⁴ and R⁵ are selected independently from hydrogen, C₁₋₅alkyl, C₁₋₅haloalkyl, C₂₋₅alkenyl, C₂₋₅alkynyl, C₃₋₆cycloalkyl, C₅₋₆arylC₁₋₂alkyl and C₅₋₆heteroarylC₁₋₂alkyl, and may be substituted by one or more groups selected independently from halogen, hydroxyl, cyano and C₁₋₅alkoxy, or
R⁴ and R⁵ form together a C₃₋₇heterocycloalkyl, and may be substituted by one or more groups selected independently from hydrogen, halogen, C₁₋₆alkyl, C₁₋₆haloalkyl, COR¹², OR¹², SO₂R¹², SO₂N(R¹¹)₂, C₅₋₆aryl, C₅₋₆heteroaryl, cyano, and oxo substituted on β or γ position;
R⁶ is hydrogen, C₁₋₆alkyl, C₃₋₆cycloakyl, R⁷OC₁₋₆alkyl, C₁₋₆haloalkyl, C₁₋₆cyanoalkyl, (R¹¹)₂NCOC₀₋₆alkyl or R¹²SO₂C₁₋₆alkyl;
R⁷ is C₁₋₁₀alkyl, C₆₋₁₀arylC₀₋₆alkyl, C₅₋₆heteroarylC₀₋₆alkyl, C₃₋₇cycloalkylC₀₋₆alkyl or C₁₋₆haloalkyl;
R⁸ is hydrogen, C₁₋₁₀alkyl, C₁₋₆haloalkyl, C₃₋₇cycloalkylC₀₋₆alkyl, C₆₋₁₀arylC₀₋₆alkyl or C₅₋₆heteroarylC₀₋₆alkyl, or
R⁷ and R⁸ form together a C₅₋₆heteroaryl or C₃₋₇heterocycloalkyl,
whereby any aryl and heteroaryl under R¹, R⁷ and R⁸ may be substituted by one or more groups selected independently from hydrogen, halogen, hydroxy, C₁₋₆haloalkyl, cyano, OR¹²C₁₋₆alkyl, oxo, SR¹¹, CON(R¹¹)₂, N(R¹¹)COR¹², SO₂R¹², SOR¹², N(R¹¹)₂ and COR¹²;
   R⁹ is hydrogen, halogen, hydroxy, C₁₋₆alkoxy, C₁₋₆haloalkoxy, C₁₋₆haloalkyl, C₁₋₆alkyl or COR¹²;
   R¹⁰ is hydrogen, C₁₋₆alkyl, C₁₋₆alkoxy or C₁₋₆haloalkyl;
   R¹¹ is hydrogen, C₁₋₆alkyl or C₁₋₆haloalkyl; and
   R¹² is C₁₋₆alkyl or C₁₋₆haloalkyl, or
   R¹¹ and R¹² form together a C₃₋₇cycloalkyl or C₃₋₇heterocycloalkyl, whereby R¹¹ and R¹² may be substituted by one or more groups selected independently from hydrogen, halogen, hydroxy, cyano, C₁₋₃alkyl, C₁₋₃alkoxy and C₁₋₃haloalkyl,
   or salts, solvates or solvated salts thereof.

Another embodiment of the invention relates to compounds of formula I wherein:

wherein:
P is C₆₋₁₀arylC₀₋₃alkyl, C₅₋₁₁heteroarylC₀₋₃alkyl or C₃₋₇cycloalkylC₀₋₃alkyl;
R¹ is hydrogen, halogen, C₁₋₁₀alkoxy, C₁₋₆haloalkyl or R⁷OC₀₋₆alkyl;
n is 0, 1, 2 or 3;
X is a single bond, O or NR⁶, or X is N in a C₅₋₁₂heteroaryl;

### Q is CH or O;

R² is hydrogen or halogen;

R³ is hydrogen, C₁₋₁₀alkyl or C₁₋₁₀alkoxy;

R⁴ and R⁵ are selected independently from hydrogen, C₁₋₅alkyl and C₁₋₅haloalkyl, or
R⁴ and R⁵ form together a C₃₋₇heterocycloalkyl, and may be substituted by one or more groups selected independently from hydrogen, C₅₋₆aryl and C₅₋₆heteroaryl;
R⁶ is hydrogen or C₁₋₆cyanoalkyl;
R⁷ is C₁₋₁₀alkyl or C₃₋₇cycloalkylC₀₋₄alkyl;
R⁹ is hydrogen; and
R¹⁰ is hydrogen;
or salts, solvates or solvated salts thereof.

In a further embodiment of the invention P is phenyl or naphtyl.

In yet another embodiment of the invention P is pyridinyl, pyrimidyl, quinoline, iso-quinoline, cyclohexyl or 1,2-methylenedioxybenzene.

The invention also relates to compounds of formula I wherein P is tetraline, chromane or indane.

In another embodiment of the invention P is substituted with 0, 1, 2, 3 or 4 groups R¹, wherein the number of R¹ substituents is designated by the term n. In another embodiment of the invention n is 0, 1 or 2.

Where P is substituted by more than one R¹ group it is to be understood that the R¹ substituent may be the same or different.

In a further embodiment of the invention R¹ is hydrogen, chloro, fluoro, bromo, methoxy, ethoxy or propoxy.

In another embodiment R¹ is C₁₋₆haloalkyl or C₁₋₆haloalkylO. In yet another embodiment R¹ is fluoromethyl, difluoromethyl, trifluoromethyl, fluoromethoxy, difluoromethoxy, trifluoromethoxy or cyano.

In one embodiment of the invention R³ is methyl, ethyl, methoxy, ethoxy or propoxy. In another embodiment R³ is hydrogen, halogen, C₁₋₆haloalkyl or C₁₋₆haloalkylO. In yet another embodiment R³ is fluoromethyl, difluoromethyl, trifluoromethyl, fluoromethoxy, difluoromethoxy or trifluoromethoxy.

In a further embodiment X is NR⁶ or O. In yet a further embodiment X is N in a C₈₋₁₂heteroaryl. In one embodiment X is N in an indol, indoline, tetrahydroquinoline, tetrahydroisoquinoline, benzoxazepine, isoindoline, pyrrole, oxindole or benzazepine.

In one embodiment of the invention R⁴ and R⁵ are selected independently from C₁₋₃alkyl and C₁₋₃haloalkyl. In another embodiment R⁴ and R⁵ are selected independently from hydrogen, methyl, ethyl, i-propyl, n-propyl and fluoroethyl.

In a further embodiment R⁴ and R⁵ form together a C₅₋₆heterocycloalkyl ring. In yet a further embodiment R⁴ and R⁵ form together a pyrrolidine.

In another embodiment R⁴ and R⁵ form together morpholine, aminolactam optionally substituted on the lactam nitrogen or N-substituted piperazine, whereby the substituent on the piperazine nitrogen may be selected independently from hydrogen, C₁₋₆alkyl, C₅₋₆aryl, C₅₋₆heteroaryl, COR¹², SO₂R¹² and SO₂N(R¹¹)₂.

In one embodiment R⁶ is hydrogen, C₁₋₆ alkyl or C₁₋₆cyanoalkyl. In a further embodiment R⁶ is hydrogen, methyl, cyanomethyl or fluoroethyl.

Another embodiment of the invention relates to compounds selected from the group consisting of
(6S)—N-(5-Chloro-2-methoxyphenyl)-6-(dimethylamino)-4-methoxy-5,6,7,8-tetrahydronaphthalene-1-sulfonamide,
(6S)-6-(Dimethylamino)-4-methoxy-N-phenyl-5,6,7,8-tetrahydronaphthalene-1-sulfonamide,
(6S)—N-(3,5-Dichloro-2-methoxyphenyl)-6-(dimethylamino)-4-methoxy-5,6,7,8-tetrahydronaphthalene-1-sulfonamide,
(6S)-6-(Dimethylamino)-N-(3-fluorophenyl)-4-methoxy-5,6,7,8-tetrahydronaphthalene-1-sulfonamide,
(6R)-6-(Dimethylamino)-4-methoxy-N-phenyl-5,6,7,8-tetrahydronaphthalene-1-sulfonamide,
(6R)-6-(Dimethylamino)-N-(3-fluorophenyl)-4-methoxy-5,6,7,8-tetrahydronaphthalene-1-sulfonamide,
(6R)—N-(5-Chloro-2-methoxyphenyl)-6-(dimethylamino)-4-methoxy-5,6,7,8-tetrahydronaphthalene-1-sulfonamide,
(6S)—N-(3,5-Dichlorophenyl)-6-(dimethylamino)-4-methoxy-5,6,7,8-tetrahydronaphthalene-1-sulfonamide,
(6S)—N-(3-Chloro-4-fluorophenyl)-6-(dimethylamino)-4-methoxy-5,6,7,8-tetrahydronaphthalene-1-sulfonamide,
(6S)-6-(Dimethylamino)-N-(6-fluoropyridin-3-yl)-4-methoxy-5,6,7,8-tetrahydronaphthalene-1-sulfonamide,
(6S)-6-(Dimethylamino)-4-methoxy-N-[(2S)-8-methoxy-1,2,3,4-tetrahydronaphthalen-2-yl]-5,6,7,8-tetrahydronaphthalene-1-sulfonamide,
(6S)—N-(3,5-Dichlorophenyl)-6-[isopropyl(methyl)amino]-4-methoxy-5,6,7,8-tetrahydronaphthalene-1-sulfonamide,
(6S)—N-(5-Chloro-2-methoxyphenyl)-4-methoxy-6-pyrrolidin-1-yl-5,6,7,8-tetrahydronaphthalene-1-sulfonamide,
(6S)—N-(3,5-Dichlorophenyl)-4-methoxy-6-pyrrolidin-1-yl-5,6,7,8-tetrahydronaphthalene-1-sulfonamide,
(6S)—N-(3-Chloro-4-fluorophenyl)-4-methoxy-6-morpholin-4-yl-5,6,7,8-tetrahydronaphthalene-1-sulfonamide,
(6S)-4-Methoxy-6-(methylamino)-N-phenyl-5,6,7,8-tetrahydronaphthalene-1-sulfonamide,
(6S)-6-(Dimethylamino)-4-methoxy-N-pyrimidin-2-yl-5,6,7,8-tetrahydronaphthalene-1-sulfonamide,
(6S)-6-(Dimethylamino)-4-methoxy-N-pyridin-2-yl-5,6,7,8-tetrahydronaphthalene-1-sulfonamide,
(6S)-6-(Dimethylamino)-4-methoxy-N-quinolin-2-yl-5,6,7,8-tetrahydronaphthalene-1-sulfonamide,
4-Methoxy-6-pyrrolidin-1-yl-5,6,7,8-tetrahydro-naphthalene-1-sulfonic acid 3,4-dichloro-phenyl ester,
[5-(3,4-Dihydro-1H-isoquinoline-2-sulfonyl)-8-methoxy-1,2,3,4-tetrahydro-naphthalen-2-yl]-dimethyl-amine,
(6S)—N-cyclohexyl-6-(dimethylamino)-4-methoxy-5,6,7,8-tetrahydronaphthalene-1-sulfonamide,
(6S)—N-(3-Chloro-4-fluorophenyl)-4-methoxy-6-pyrrolidin-1-yl-5,6,7,8-tetrahydronaphthalene-1-sulfonamide,
(6S)—N-(5-chloro-2-methoxyphenyl)-N-(cyanomethyl)-4-methoxy-6-pyrrolidin-1-yl-5,6,7,8-tetrahydronaphthalene-1-sulfonamide,
(6S)—N-(4-chlorophenyl)-4-methoxy-6-(methylamino)-5,6,7,8-tetrahydronaphthalene-1-sulfonamide,
(6S)-4-methoxy-6-pyrrolidin-1-yl-N-[3-(trifluoromethyl)phenyl]-5,6,7,8-tetrahydronaphthalene-1-sulfonamide,
(6S)-4-methoxy-N-phenyl-6-pyrrolidin-1-yl-5,6,7,8-tetrahydronaphthalene-1-sulfonamide,
(6S)-6-[(2-fluoroethyl)amino]-4-methoxy-N-phenyl-5,6,7,8-tetrahydronaphthalene-1-sulfonamide,
(2S)-5-(2,3-dihydro-1H-indol-1-ylsulfonyl)-8-methoxy-N,N-dimethyl-1,2,3,4-tetrahydronaphthalen-2-amine,
(6S)—N-(5-chloro-2-methoxyphenyl)-4-methoxy-6-(methylamino)-5,6,7,8-tetrahydronaphthalene-1-sulfonamide,
(6S)—N-(4-chlorophenyl)-6-(dimethylamino)-4-methoxy-5,6,7,1-tetrahydronaphthalene-1-sulfonamide,
2-{[(6S)-4-methoxy-6-pyrrolidin-1-yl-5,6,7,8-tetrahydronaphthalen-1-yl]sulfonyl}-1,2,3,4-tetrahydroisoquinoline-7-carbonitrile,
(6S)—N-(4-chlorophenyl)-4-methoxy-6-pyrrolidin-1-yl-5,6,7,8-tetrahydronaphthalene-1-sulfonamide,
(6S)—N-(3,4-dichlorophenyl)-4-methoxy-6-pyrrolidin-1-yl-5,6,7,8-tetrahydronaphthalene-1-sulfonamide,
(6S)—N-(3,4-difluorophenyl)-4-methoxy-6-pyrrolidin-1-yl-5,6,7,8-tetrahydronaphthalene-1-sulfonamide,
(6S)—N-(5-chloropyridin-2-yl)-6-(dimethylamino)-4-methoxy-5,6,7,8-tetrahydronaphthalene-1-sulfonamide,
(6S)-6-(dimethylamino)-4-methoxy-N-pyridin-3-yl-5,6,7,8-tetrahydronaphthalene-1-sulfonamide,
(6S)—N-1,3-benzodioxol-5-yl-4-methoxy-6-pyrrolidin-1-yl-5,6,7,8-tetrahydronaphthalene-1-sulfonamide,
(6S)—N-(5-chloro-2-methoxyphenyl)-6-[(2-fluoroethyl)amino]-4-methoxy-5,6,7,8-tetrahydronaphthalene-1-sulfonamide,
(6S)—N-(5-chloro-2-methoxyphenyl)-6-[(2-fluoroethyl)(methyl)amino]-4-methoxy-5,6,7,8-tetrahydronaphthalene-1-sulfonamide,
(6S)-4-methoxy-6-(methylamino)-N-[4-(trifluoromethyl)phenyl]-5,6,7,8-tetrahydronaphthalene-1-sulfonamide,
(6S)—N-(4-chlorophenyl)-4-methoxy-N-methyl-6-(methylamino)-5,6,7,8-tetrahydronaphthalene-1-sulfonamide,
(2S)-5-(1H-indol-1-ylsulfonyl)-8-methoxy-N-methyl-1,2,3,4-tetrahydronaphthalen-2-amine,
(2S)-5-[(5-chloro-1H-indol-1-yl)sulfonyl]-8-methoxy-N-methyl-1,2,3,4-tetrahydronaphthalen-2-amine,
(2S)-8-methoxy-N-methyl-5-{[6-(trifluoromethyl)-1H-indol-1-yl]sulfonyl}-1,2,3,4-tetrahydronaphthalen-2-amine,
1-{[(6S)-4-methoxy-6-(methylamino)-5,6,7,8-tetrahydronaphthalen-1-yl]sulfonyl}-1H-indole-6-carbonitrile,
(2S)-5-[(7-fluoro-1H-indol-1-yl)sulfonyl]-8-methoxy-N-methyl-1,2,3,4-tetrahydronaphthalen-2-amine,
(2S)-5-[(4-fluoro-1H-indol-1-yl)sulfonyl]-8-methoxy-N-methyl-1,2,3,4-tetrahydronaphthalen-2-amine,
(2S)-8-methoxy-5-[(4-methoxy-1H-indol-1-yl)sulfonyl]-N-methyl-1,2,3,4-tetrahydronaphthalen-2-amine,
(2S)-5-(5H-[1,3]dioxolo[4,5-f]indol-5-ylsulfonyl)-8-methoxy-N-methyl-1,2,3,4-tetrahydronaphthalen-2-amine,
(2S)-5-[(7-chloro-1H-indol-1-yl)sulfonyl]-8-methoxy-N-methyl-1,2,3,4-tetrahydronaphthalen-2-amine,
(2S)-8-methoxy-N-methyl-5-(1H-pyrrolo[2,3-b]pyridin-1-ylsulfonyl)-1,2,3,4-tetrahydronaphthalen-2-amine,
(6S)-6-(dimethylamino)-4-methoxy-N-quinolin-3-yl-5,6,7,8-tetrahydronaphthalene-1-sulfonamide,
(6S)-6-(dimethylamino)-N-isoquinolin-3-yl-4-methoxy-5,6,7,8-tetrahydronaphthalene-1-sulfonamide,
(6S)—N-1,3-benzothiazol-6-yl-6-(dimethylamino)-4-methoxy-5,6,7,8-tetrahydronaphthalene-1-sulfonamide,
(2S)-5-[(3-chloro-1H-pyrrolo[2,3-b]pyridin-1-yl)sulfonyl]-8-methoxy-N,N-dimethyl-1,2,3,4-tetrahydronaphthalen-2-amine,
(2S)-5-(1H-benzimidazol-1-ylsulfonyl)-8-methoxy-N,N-dimethyl-1,2,3,4-tetrahydronaphthalen-2-amine,
(6S)—N-(4-cyanophenyl)-4-methoxy-6-(methylamino)-5,6,7,8-tetrahydronaphthalene-1-sulfonamide, and
(6S)-6-(methylamino)-N-[4-(trifluoromethyl)phenyl]-5,6,7,8-tetrahydronaphthalene-1-sulfonamide,
   or salts, solvates or solvated salts thereof.

A further embodiment of the invention relates to compounds selected from the group
(3R)—N-(5-Chloro-2-methoxyphenyl)-3-(dimethylamino)-5-methoxychromane-8-sulfonamide,
(3R)—N-(5-Chloro-2-methoxyphenyl)-3-(diethylamino)-5-methoxychromane-8-sulfonamide,
(3R)—N-(5-Chloro-2-methoxyphenyl)-3-(dipropylamino)-5-methoxychromane-8-sulfonamide,
(3R)—N-(5-Chloro-2-methoxyphenyl)-5-methoxy-3-pyrrolidin-1-ylchromane-8-sulfonamide,
(3R)—N-(3-Chloro-4-fluorophenyl)-3-(dimethylamino)-5-methoxychromane-8-sulfonamide,
(3R)—N-(3-Chloro-4-fluorophenyl)-3-(isopropylamino)-5-methoxychromane-8-sulfonamide,
(3R)—N-(3-Chloro-4-fluorophenyl)-3-[isopropyl(methyl)amino]-5-methoxychromane-8-sulfonamide,
(3R)—N-(3-Chloro-4-fluorophenyl)-5-methoxy-3-pyrrolidin-1-ylchromane-8-sulfonamide,
(3R)—N-(3,5-Dichlorophenyl)-3-(dimethylamino)-methoxychromane-8-sulfonamide,
(3R)—N-(3,5-Dichlorophenyl)-5-methoxy-3-pyrrolidin-1-ylchromane-8-sulfonamide,
(3R)-3-(Dimethylamino)-5-methoxy-N-phenylchromane-8-sulfonamide,
(3R)-5-Methoxy-3-(methylamino)-N-phenylchromane-8-sulfonamide,
(3R)—N-(3-Chloro-4-fluorophenyl)-3-(dimethylamino)-5-ethylchromane-8-sulfonamide,
(3R)-6-Chloro-N-phenyl-3-pyrrolidin-1-ylchromane-8-sulfonamide,
(3R)—N-(4-Chlorophenyl)-5-methoxy-3-(methylamino)chromane-8-sulfonamide,
(3R)-5-Methoxy-3-(methylamino)-N-[4-(trifluoromethyl)phenyl]chromane-8-sulfonamide,
(3R)—N-(3,4-Dichlorophenyl)-5-methoxy-3-(methylamino)chromane-8-sulfonamide,
(3R)-5-Methoxy-3-(methylamino)-N-[3-(trifluoromethyl)phenyl]chromane-8-sulfonamide,
(3R)-5-Methoxy-3-(methylamino)-N-quinolin-2-ylchromane-8-sulfonamide,
(3R)—N-(3-Cyanophenyl)-5-methoxy-3-(methylamino)chromane-8-sulfonamide,
(3R)—N-(4-Cyanophenyl)-5-methoxy-3-(methylamino)chromane-8-sulfonamide,
(3R)—N-(4-Chlorophenyl)-3-(dimethylamino)-5-methoxychromane-8-sulfonamide,
(3R)—N-(3-Cyanophenyl)-3-(dimethylamino)-5-methoxychromane-8-sulfonamide, and
(3R)—N-(4-Cyanophenyl)-3-(dimethylamino)-5-methoxychromane-8-sulfonamide,
   or salts, solvates or solvated salts thereof.

Listed below are definitions of various terms used in the specification and claims to describe the present invention.

For the avoidance of doubt it is to be understood that where in this specification a group is qualified by ‘hereinbefore defined’, ‘defined hereinbefore’ or ‘defined above’ the said group encompasses the first occurring and broadest definition as well as each and all of the other definitions for that group.

For the avoidance of doubt it is to be understood that in this specification ‘C₁₋₆’ means a carbon group having 1, 2, 3, 4, 5 or 6 carbon atoms.

In this specification, unless stated otherwise, the term “alkyl” includes both straight and branched chain alkyl groups and may be, but are not limited to methyl, ethyl, n-propyl, i-propyl, n-butyl, i-butyl, s-butyl, t-butyl, n-pentyl, i-pentyl, neo-pentyl, n-hexyl or i-hexyl. The term C₁₋₄ allyl having 1 to 4 carbon atoms and may be but are not limited to methyl, ethyl, n-propyl, i-propyl or tert-butyl.

The term ‘C₀’ means a bond or does not exist. For example when “arylC₀alkyl” is equivalent with “aryl”, “C₂alkylOC₀alkyl” is equivalent with “C₂alkylO”.

In this specification, unless stated otherwise, the term “alkenyl” includes both straight and branched chain alkenyl groups. The term “C₂₋₆alkenyl” having 2 to 6 carbon atoms and one or two double bonds, may be, but is not limited to vinyl, allyl, propenyl, butenyl, crotyl, pentenyl, or hexenyl, and a butenyl group may for example be buten-2-yl, buten-3-yl or buten-4-yl.

In this specification, unless stated otherwise, the term “alkynyl” includes both straight and branched chain alkynyl groups. The term “C₂₋₆alkynyl” having 2 to 6 carbon atoms and one or two trippel bonds, may be, but is not limited to ethynyl, propargyl, pentynyl or hexynyl and a butynyl group may for example be butyn-3-yl or butyn-4-yl.

The term “alkoxy”, unless stated otherwise, refers to radicals of the general formula —O—R, wherein R is selected from a hydrocarbon radical. The term “alkoxy” may include, but is not limited to methoxy, ethoxy, propoxy, isopropoxy, butoxy, t-butoxy, isobutoxy, cyclopropylmethoxy, allyloxy or propargyloxy.

In this specification, unless stated otherwise, the term “amine” or “amino” refers to radicals of the general formula —NRR′, wherein R and R′ are selected independently from hydrogen or a hydrocarbon radical.

In this specification, unless stated otherwise, the term “cycloalkyl” refers to an optionally substituted, partially or completely saturated cyclic hydrocarbon ring system. The term “C₃₋₇cycloalkyl” may be, but is not limited to cyclopropyl, cyclobutyl, cyclopentyl, cyclohexyl, cycloheptyl or cyclopentenyl.

The term “heterocycloalkyl” denotes a non-aromatic, partially or completely saturated hydrocarbon group, which contains at least one ring and at least one heteroatom. Examples of said heterocycle include, but are not limited to pyrrolidinyl, pyrrolidinonyl, piperidinyl, piperazinyl, morpholinyl, oxazolyl, 2-oxazolidinyl or tetrahydrofuranyl.

In this specification, unless stated otherwise, the term “aryl” refers to an optionally substituted monocyclic, bicyclic or tricyclic hydrocarbon ring system with at least one unsaturated aromatic ring. Examples of “aryl” may be, but are not limited to phenyl, naphthyl or tetralinyl.

In this specification, unless stated otherwise, the term “heteroaryl” refers to an optionally substituted monocyclic, bicyclic or tricyclic hydrocarbon ring system with at least one unsaturated ring and containing at least one heteroatom selected independently from N, O or S. Examples of “heteroaryl” may be, but are not limited to pyridinyl, pyrrolyl, furyl, thienyl, imidazolyl, oxazolyl, isoxazolyl, thiazolyl, pyrazolyl, benzofuryl, indolyl, indolinyl isoindolyl, benzimidazolyl, pyridazinyl, pyrimidinyl, pyrazinyl, pyrrolo[2,3-b]pyridinyl, benzimidazolyl, 1,2,3,4-tetrahydroquinolinyl, 1,2,3,4-tetrahydroisoquinolinyl, 1,3-benzothiazolyl, imidazo[2,1-b][1,3]thiazolyl, quinolinyl, isoquinolinyl, benzothiophenyl, benzoxadiazolyl, 1,3-benzodioxolyltetrazolyl, triazolyl, quinazolinyl or isothiazolyl. For the avoidance of doubt, a C₅heteroaryl refers to a 5 membered aromatic ring system containing at least one heteroatom.

In this specification, unless stated otherwise, the terms “arylalkyl” and “heteroarylalkyl” refer to a substituent that is attached via the alkyl group to an aryl or heteroaryl group.

In this specification, unless stated otherwise, the terms “halo” and “halogen” may be fluoro, iodo, chloro or bromo.

In this specification, unless stated otherwise, the term “haloalkyl” means an alkyl group as defined above, which is substituted with halo as defined above. The term “C₁₋₆haloalkyl” may include, but is not limited to fluoromethyl, difluoromethyl, trifluoromethyl, fluoroethyl, difluoroethyl or bromopropyl. The term “C₁₋₆haloalkylO” may include, but is not limited to fluoromethoxy, difluoromethoxy, trifluoromethoxy, fluoroethoxy or difluoroethoxy.

The present invention relates to the compounds of formula I, as hereinbefore defined as well is as to the salts, solvates or solvated salts thereof. Salts for use in pharmaceutical formulations will be pharmaceutically acceptable salts, but other salts may be useful in the production of the compounds of formula I.

A suitable pharmaceutically acceptable salt of the compounds of the invention is, for example, an acid-addition salt, for example a salt with an inorganic or organic acid. In addition, a suitable pharmaceutically acceptable salt of the compounds of the invention is an alkali metal salt, an alkaline earth metal salt or a salt with an organic base.

Other pharmaceutically acceptable salts and methods of preparing these salts may be found in, for example, Remington's Pharmaceutical Sciences (18^{th} Edition, Mack Publishing Co.).

Some compounds of formula I may have chiral centres and/or geometric isomeric centres (E- and Z-isomers), and it is to be understood that the invention encompasses all such optical, diastereoisomeric and geometric isomers.

The invention also relates to any and all tautomeric forms of the compounds of formula I.

### Methods of Preparation

One embodiment of the invention relates to processes for the preparation of the compound of formula I wherein R¹ to R¹², P, Q, X and n, unless otherwise specified, are defined as in formula I and PG is a suitable protecting group.

### Detailed Process Description:

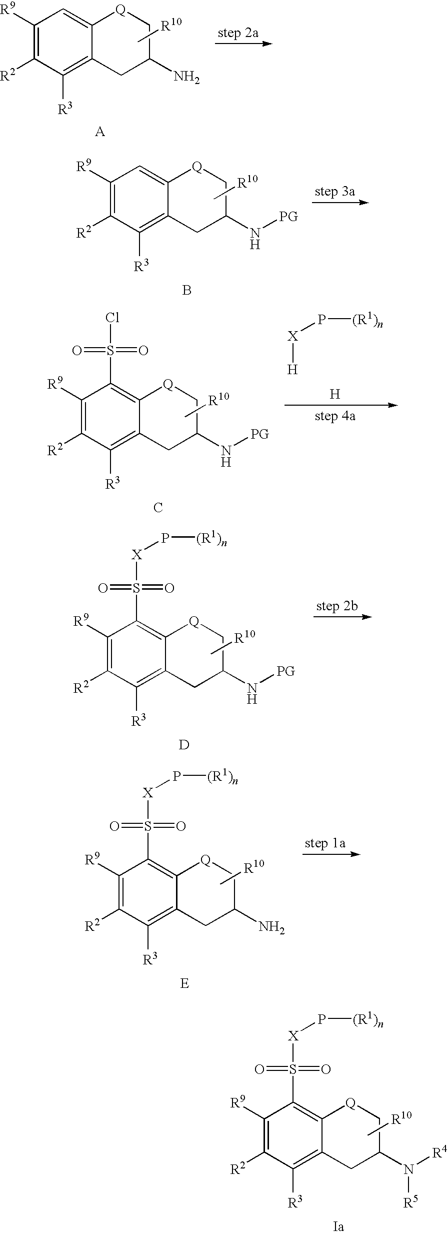

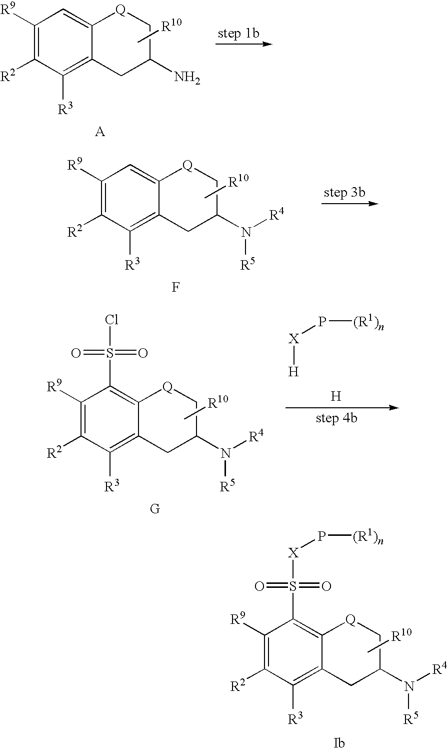

### Step 1a and 1b, E to Ia and A to F

A compound Ia may be prepared from a compound E by alkylation with a compound R⁴Y or R⁵Y where Y may be a suitable leaving group such as a halogen, mesylate or triflate, such as for example described in “Comprehensive Organic Transformations, a Guide to Functional Group Preparation”, R. C. Larock, John Wiley & sons, New York, 1999. Typically, E and R⁴Y or R⁵Y are mixed in a solvent such as DMF, ethanol, dichloromethane or toluene in the presence of a base such as sodium bicarbonate, sodium carbonate, potassium carbonate, triethylamine or diisopropylethylamine and optionally, if Y=Cl, Br, a catalytic amount of potassium iodide. The reaction may be performed at temperatures between 25° C. and the reflux temperature of the solvent and the reaction time may be between 1 and 100 hours. The reaction mixture may be either worked up by extraction and then purified by column chromatography or the reaction mixture may be concentrated and purified by column chromatography. The reaction temperature may be elevated above the reflux temperature of the solvent and reaction times shortened by the use of microwave heating. For compounds where R⁴ and R⁵ form a ring, a compound YR⁴R⁵Y may be reacted with a compound E.

Alternatively, a compound Ia may be prepared from a compound E using reductive amination. Typically E may be mixed with a carbonyl compound such as an aldehyde or a ketone in the presence of a reducing agent such as sodium borohydride, sodium cyanoborohydride, sodium triacetoxyborohydride or hydrogen in the presence of a suitable catalyst such as for example described in “Advanced Organic Chemistry, Reactions, Mechanisms and Structure”, J. March, John Wiley & Sons, New York, 1992. An acid such as formic acid or acetic acid may be added to control the pH of the reaction. The reaction may be performed in a solvent such as water, methanol, ethanol, dichloromethane, THF, formic acid, acetic acid or mixtures thereof at temperatures between 0° C. and the reflux temperature of the solvent, preferably at RT. The reaction mixture may be either worked up by extraction and then purified by column chromatography or the reaction mixture may be concentrated and purified by column chromatography.

A compound Ia may also be prepared from a compound E by first preparing the amide or carbamate followed by reduction using an appropriate reducing agent. The amide may for example be prepared by reaction of E with an acid chloride or with a carboxylic acid in the presence of a coupling reagent, such as for example described in “Comprehensive Organic Transformations, a Guide to Functional Group Preparation”, R. C. Larock, John Wiley & sons, New York, 1999. The carbamate may be prepared by the reaction of an alkylchloroformate with a compound E in a solvent such as dichloromethane in the presence of a base such as triethylamine or pyridine at temperatures between 0° C. and the reflux temperature of the solvent. The reduction of the carbamate or the amide may be performed with a reducing agent such as lithium aluminum hydride in a solvent such as tetrahydrofuran or diethyl ether at temperatures between 0° C. and the reflux temperature of the solvent, preferably between 25° C. and the reflux temperature. The reduction of the amide may also be performed using borane as the reducing agent.

The same procedures as described for the transformation of a compound E to a compound Ia may be used to transform a compound A into a compound F.

### Step 2a and 2b, A to B and D to E

A compound A may be transformed into a compound B or a compound D may be transformed into a compound E using standard protecting groups. Conventional procedures for using such protecting groups, as well as examples of suitable protecting groups are described in, for example, “Protective Groups in Organic Synthesis” T. W. Green, P. G. M. Wuts, Wiley-Interscience, New York, 1999.

### Step 3a and 3b, B to C and F to G

A compound B may be transformed into a compound C by chlorosulfonylation. Compound B may be dissolved in a solvent such as dichloromethane, chloroform or ethyl acetate and cooled to a temperature between −72° C. and 0° C. The reaction may also be run neat in chlorosulfonic acid. Chlorosulfonic acid, optionally diluted in a solvent such as chloroform or methylene chloride, may be added dropwise while cooling. The reaction may be stirred at temperatures between −72° C. and the reflux temperature of the solvent for 1 to 100 hours. Optionally a chlorinating agent such as thionyl chloride may be added to the reaction mixture. The reaction may be quenched by adding the reaction mixture to ice-water, optionally containing a base such as sodium bicarbonate and the raw product may be isolated by extraction or filtration and used without further purification or if stable enough, purified by column chromatography.

To ensure complete transformation of B into C via the sulfonic acid, the crude may be dissolved in a solvent such as chloroform or toluene and a chlorinating agent such as thionyl chloride or oxalyl chloride may be added. Optionally a catalytic amount of DMF may be added and the mixture may be heated to between 25° C. and the reflux temperature of the solvent. The workup and purification may be performed as in the previous section. The same reaction conditions may be used for the transformation of a compound F to a compound G.

### Step 4a and 4b, G to Ib and C to D

A compound Ib may be prepared by the reaction of a compound of formula H with a compound G. A compound G may be reacted with a compound H in the presence of an organic base such as pyridine, triethylamine or diisopropylethylamine or an inorganic base such as sodium hydroxide or potassium carbonate in a solvent such as dichloromethane, acetonitrile, DMF or THF at a temperature between 0° C. and the reflux temperature of the solvent, preferably at RT. The product may be isolated by column chromatography or by extraction followed by column chromatography.

Alternatively a compound G may be reacted with ammonia or a compound R⁶NH₂ in a solvent such as methanol or dioxane at temperatures between 0° C. and the reflux temperature of the solvent to form an intermediate. This intermediate may then be reacted with an electron poor aromatic or heteroaromatic compound with a halogen leaving group such as chlorine or fluorine, in an aprotic solvent such as DMF in the presence of a base such as sodium hydride at temperatures between RT and the reflux temperature of the solvent, preferably at temperatures between 70° C. and the reflux temperature of the solvent for 1-24 hours. The reaction may also be performed using microwave irradiation as a heating source.

The same procedures as described for the transformation of a compound G to a compound Ib may be used to transform a compound C into a compound D.

### Step 5a and 5b, Introduction of R⁶

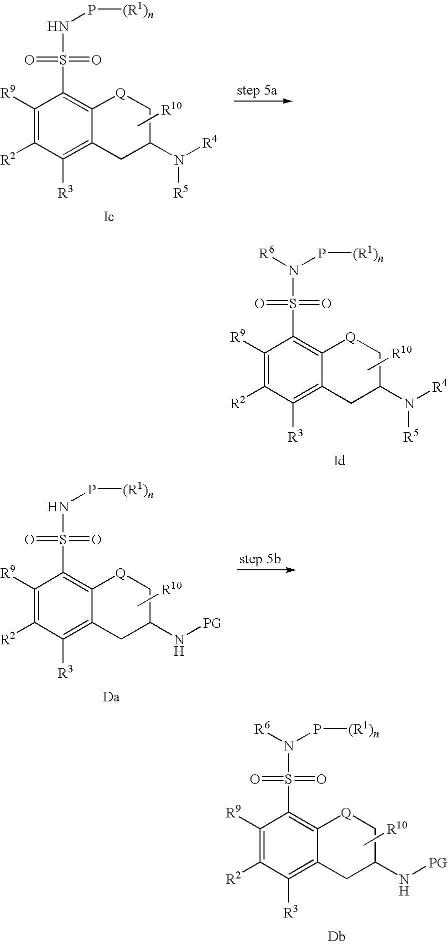

A compound Ic may be transformed into a compound Id, R⁶ may not be H, by alkylation using a compound R⁶Y where Y may be a suitable leaving group such as iodine, bromine, chlorine, mesylate or triflate. A compound Ic may be mixed with a strong base such as sodium hydride in a solvent such as DMF, THF or dioxane and R⁶Y may be added. The reaction may be performed at temperatures between RT and the reflux temperature of the solvent for 1-24 hours. The product may be isolated by column chromatography.

The same method can be used to transform a compound Da into a compound Db.

### Intermediates

A further embodiment of the invention relates to compounds selected from the group consisting of compound of

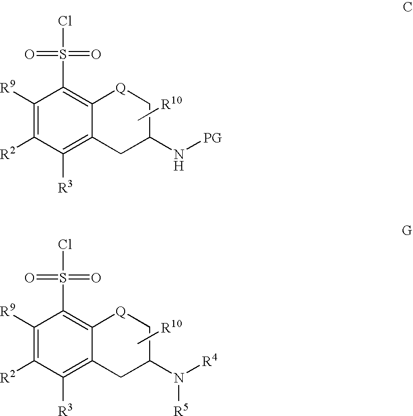

wherein R¹ to R⁹ are defined as hereinbefore and PG is a suitable leaving group,
with the proviso that R² and R⁹ are not both methyl, and
(6S)-6-(dimethylamino)-4-methoxy-5,6,7,8-tetrahydronaphthalene-1-sulfonyl chloride,
(6R)-6-(dimethylamino)-4-methoxy-5,6,7,8-tetrahydronaphthalene-1-sulfonyl chloride,
(6S)-4-methoxy-6-[(trifluoroacetyl)amino]-5,6,7,8-tetrahydronaphthalene-1-sulfonyl chloride,
(3R)-5-methoxy-3-[(trifluoroacetyl)amino]chromane-8-sulfonyl chloride,
(3R)-5-ethyl-3-[(trifluoroacetyl)amino]chromane-8-sulfonyl chloride, and
(3R)-6-chloro-3-[(trifluoroacetyl)amino]chromane-8-sulfonyl chloride,
   which may be used as intermediates in the preparation of compounds suited for the treatment of 5HT6 mediated disorders, especially for use as intermediates for the preparation of compounds of formula I.

### Pharmaceutical Composition

According to one embodiment of the present invention there is provided a pharmaceutical composition comprising as active ingredient a therapeutically effective amount of the compound of formula I, or salts, solvates or solvated salts thereof, in association with one or more pharmaceutically acceptable diluents, excipients and/or inert carriers.

The composition may be in a form suitable for oral administration, for example as a tablet, pill, syrup, powder, granule or capsule, for parenteral injection (including intravenous, subcutaneous, intramuscular, intravascular or infusion) as a sterile solution, suspension or emulsion, for topical administration e.g. as an ointment, patch or cream, for rectal administration e.g. as a suppository or for inhalation.

In general the above compositions may be prepared in a conventional manner using one or more conventional excipients, pharmaceutical acceptable diluents and/or inert carriers.

Suitable daily doses of the compounds of formula I in the treatment of a mammal, including man, are approximately 0.01 to 250 mg/kg bodyweight at peroral administration and about 0.001 to 250 mg/kg bodyweight at parenteral administration.

The typical daily dose of the active ingredient varies within a wide range and will depend on various factors such as the relevant indication, severity of the illness being treated, the route of administration, the age, weight and sex of the patient and the particular compound being used, and may be determined by a physician.

### Medical Use

Interestingly, it has been found that the compounds according to the present invention are useful in therapy. The compounds of formula I, or salts, solvates or solvated salts thereof, as well as their corresponding active metabolites, exhibit a high degree of potency and selectivity for 5-hydroxy-tryptamine 6 (5HT6) receptors. Accordingly, the compounds of the present invention are expected to be useful in the treatment of conditions associated with altered activation of 5HT6 receptors.

The compounds may be used to produce a modulating effect of 5HT6 receptors in mammals, including man.

The compounds of formula I are expected to be suitable for the treatment of disorders relating to or affected by the 5HT6 receptor including cognitive, personality, behaviour, psychiatric and neurodegenerative disorders.

Examples of such disorder may be selected from the group comprising of Alzheimer's disease anxiety, depression, convulsive disorders such as epilepsy, personality disorders, obsessive compulsive disorders, migraine, cognitive disorders such as memory dysfunction, sleep disorders, feeding disorders such as anorexia, obesity, bulimia, panic attacks, withdrawal from drug abuse, schizophrenia, attention deficit hyperactive disorder (ADHD), attention deficit disorder (ADD), dementia, memory loss, disorders associated with spinal trauma and/or head injury, stroke, diabetes type 2, binge disorders, bipolar disorders, psychoses, Parkinson's disease, Huntington's disease, neurodegenerative disorders characterized by impaired neuronal growth, and pain.

Further relevant disorders may be selected from the group comprising gastrointestinal disorders such as gastro-esophageal reflux disease (GERD) and irritable bowel syndrome (IBS).

The compounds may also be used for treatment of tolerance to 5HT6 activators.

One embodiment of the invention relates to the compounds of formula I as hereinbefore defined, for use in therapy.

Another embodiment of the invention relates to the compounds of formula I as hereinbefore defined, for use in treatment of 5HT6 mediated disorders.

A further embodiment of the invention relates to the compounds of formula I as hereinbefore defined, for use in treatment of Alzheimer's disease.

Another embodiment of the invention relates to the compounds of formula I as hereinbefore defined, for use in treatment of cognitive impairment associated with schizophrenia.

Yet a further embodiment of the invention relates to the compounds of formula I as hereinbefore defined, for use in treatment of obesity.

One embodiment of the invention relates to the compounds of formula I as hereinbefore defined, for use in Parkinson's disease.

Another embodiment of the invention relates to the use of the compounds of formula I as hereinbefore defined, in the manufacture of a medicament for treatment of 5HT6 mediated disorders, Alzheimer's disease, cognitive impairment associated with schizophrenia, obesity and/or Parkinson's disease, and any other disorder mentioned above.

A further embodiment of the invention relates to a method of treatment of 5HT6 mediated disorders, Alzheimer's disease, cognitive impairment associated with schizophrenia, obesity and/or Parkinson's disease, and any other disorder mentioned above, comprising administering to a mammal, including man in need of such treatment, a therapeutically effective amount of the compounds of formula I, as hereinbefore defined.

Yet another embodiment of the invention relates to a pharmaceutical composition comprising a compound of formula I as hereinbefore defined, for use in treatment of 5HT6 mediated disorders, Alzheimer's disease, cognitive impairment associated with schizophrenia, obesity and/or Parkinson's disease, and any other disorder mentioned above.

One embodiment of the invention relates to an agent for the prevention or treatment of 5HT6 mediated disorders, Alzheimer's disease, cognitive impairment associated with schizophrenia, obesity and/or Parkinson's disease, and any other disorder mentioned above, which comprises as active ingredient a compound of formula I as hereinbefore defined.

In the context of the present specification, the term “therapy” and “treatment” includes prevention and prophylaxis, unless there are specific indications to the contrary. The terms “treat”, “therapeutic” and “therapeutically” should be construed accordingly.

In this specification, unless stated otherwise, the terms “inhibitor” and “antagonist” mean a compound that by any means, partly or completely, blocks the transduction pathway leading to the production of a response by the agonist.

The compounds according to the present invention are modulators of the 5HT6 receptors, and may be inhibitors, as well as agonists, inverse-agonists or partial-agonist.

The term “disorder”, unless stated otherwise, means any condition and disease associated with 5HT6 receptor activity.

### Non-Medical Use

In addition to their use in therapeutic medicine, the compounds of formula I, or salts, solvates or solvated salts thereof, are also useful as pharmacological tools in the development and standardisation of in vitro and in vivo test systems for the evaluation of the effects of modulators of 5HT6 related activity in laboratory animals such as cats, dogs, rabbits, monkeys, rats and mice, as part of the search for new therapeutics agents.

### EXAMPLES

### General Methods

The invention will now be illustrated by the following Examples in which, generally:
(i) operations were carried out at ambient or room temperature, i.e. in the range 17 to 25° C. and under an atmosphere of an inert gas such as argon unless otherwise stated;
(ii) evaporations were carried out by rotary evaporation under reduced pressure and work-up procedures were carried out after removal of residual solids by filtration;
(iii) HPLC analyses were performed on an Agilent HP 1000 system consisting of G1379A Micro Vacuum Degasser, G1312A Binary Pump, G1367A Wellplate auto-sampler, G1316A Thermostatted Column Compartment and G1315B Diode Array Detector. Column: X-Terra MS, Waters, 4.6×50 mm, 3.5 μm. The column temperature was set to 40° C. and the flow rate to 1.5 ml/min. The Diode Array Detector was scanned from 210-300 nm, step and peak width were set to 2 nm and 0.05 min, respectively. A linear gradient was applied, run from 0% to 100% acetonitrile, in 4 min. Mobile phase: acetonitrile/10 mM ammonium acetate in 5% acetonitrile in MilliQ Water.
(iv) Thin layer chromatography (TLC) was performed on Merck TLC-plates (Silica gel 60 F₂₅₄) and UV visualized the spots. Flash chromatography was preformed on a Combi Flash® Companion™ using RediSep™ normal-phase flash columns or on Merck Silica gel 60 (0.040-0.063 mm). Typical solvents used for flash chromatography were mixtures of chloroform/methanol, methylenechloride/methanol, chloroform/methanol/ammonia, toluene/ethyl acetate and ethyl acetate/heptane.
(v) ¹H and ¹³C NMR spectra were recorded at 400 MHz for proton and 100 MHz for carbon-13 either on a Varian Unity+ 400 NMR Spectrometer equipped with a 5 mm BBO probe with Z-gradients, or a Bruker Avance 400 NMR spectrometer equipped with a 60 μl dual inverse flow probe with Z-gradients, or a Bruker DPX400 NMR spectrometer equipped with a 4-nucleus probe equipped with Z-gradients. The following reference signals were used: the middle line of DMSO-d₆ δ 2.50 (¹H); the middle line of CD₃OD δ 3.31 (¹H); acetone-d₆ 2.04(¹H); and CDCl₃ δ 7.26 (¹H) (unless otherwise indicated);
(vi) Mass spectra were recorded on a Waters LCMS consisting of an Alliance 2795 (LC) and a ZQ single quadrupole mass spectrometer. The mass spectrometer was equipped with an electrospray ion source (ESI) operated in a positive or negative ion mode. The capillary voltage was 3 kV and the mass spectrometer was scanned from m/z 100-700 with a scan time of 0.3 or 0.8 s. Separations were performed on either Waters X-Terra MS, C8-columns, (3.5 μm, 50 or 100 mm×2.1 mm i.d.), or a ScantecLab's ACE 3 AQ column (100 mm×2.1 mm i.d.). The column temperature was set to 40° C. A linear gradient was applied using a neutral or acidic mobile phase system, running at 0% to 100% organic phase in 4-5 minutes, flow rate 0.3 ml/min. Mobile phase system: acetonitrile/[10 mM NH₄OAc (aq.)/MeCN (95:5)], or [10 mM NH₄OAc (aq.)/MeCN (1/9)]/[10 m OAc(aq.)/MeCN(9/1)]. Acidic mobile phase system: [133 mM HCOOH(aq.)/MeCN(5/95)]/[8 mM HCOOH(aq.)/MeCN(98/2)];
(vii) Alternatively a LC-MS system (Sample Manager 2777C, 1525μ binary pump, 1500 Column Oven, ZQ, PDA2996 and ELS detector, Sedex 85) from Waters was used. Separation was performed using a Zorbax column (CS, 3.0×50 mm, 3 μm). A four minutes linear gradient was used starting at 100% A (A=10 mM NH4OAc in 5% MeOH) and ending at 100% B (MeOH). The ZQ was equipped with a combined APPI/APCI ion source and scanned in the positive mode between m/z 120-800 with a scan time of 0.3 s. The APPI repeller and the APCI corona were set to 0.86 kV and 0.80 μA, respectively. In addition, the desolvation temperature (300° C.), desolvation gas (400 L/Hr) and cone gas (5 L/Hr) were constant for both APCI and APPI mode;
(viii) Preparative chromatography was run on a Gilson auto-preparative HPLC with a diode array detector. Column: XTerra MS C8, 19×300 mm, 7 μm. Gradient with acetonitrile/0.1M ammonium acetate in 5% acetonitrile in MilliQ Water, run from 20% to 60% acetonitrile, in 13 min. Flow rate: 20 ml/min. Alternatively, purification was achieved on a semi preparative Shimadzu LC-8A HPLC with a Shimadzu SPD-10A UV-vis.-detector equipped with a Waters Symmetry® column (C18, 5 μm, 100 mm×19 mm). Gradient with acetonitrile/0.1% trifluoroacetic acid in MilliQ Water, run from 35% to 60% acetonitrile in 20 min. Flow rate: 10 ml/min;
(ix) All solvents used were analytical grade and commercially available anhydrous solvents for reactions. Reactions were typically run under an inert atmosphere of nitrogen or argon;
(x) yields, where present, are not necessarily the maximum attainable;
(xi) intermediates were not necessarily fully purified but their structures and purity were assessed by thin layer chromatographic, HPLC, infra-red (IR), MS and/or NMR analysis;
(xii) melting points are uncorrected and were determined using a Mettler SP62 automatic melting point apparatus or an oil-bath apparatus; melting points for the end-products of the Formula I were determined after crystallisation from an appropriate organic solvent or solvent mixture;
(xiii) the following abbreviations have been used:
   HPLC high performance liquid chromatography
   LC liquid chromatography
   MS mass spectrometry
   ret. time retention time
   TFA trifluoroacetic acid
   THF tetrahydrofurane
   DMF dimethylformamide

### DIPEA N,N-diisopropylethylamine

DMSO dimethylsulfoxide
NMP 1-methyl-2-pyrrolidinone
THF tetrahydrofuran
MeOH methanol
RT room temperature
EtOAc ethyl acetate
LAH lithium aluminumhydride

Throughout the following description of such processes it is understood that, where appropriate, suitable protecting groups will be added to, and subsequently removed from, the various reactants and intermediates in a manner that will be readily understood by one skilled in the art of organic synthesis. The specific sequence of reactions depicted is not critical. For many of the compounds described the order of the reaction steps may be varied.

The invention will now be illustrated by the following non-limiting examples.

Starting materials were prepared according to the following references:
(2S)-8-methoxy-N,N-dimethyl-1,2,3,4-tetrahydronaphthalen-2-amine and (2R)-8-Methoxy-N,N-dimethyl-1,2,3,4-tetrahydronaphthalen-2-amine (J. Med. Chem. 1989, 32, 779-783),
(2S)-8-methoxy-1,2,3,4-tetrahydronaphthalen-2-amine hydrochloride (Acta Chem. Scand., Ser. B 1988, 42, 231-236),
(3R)-5-methoxy-3-[(trifluoroacetyl)amino]chromane (J. Med. Chem. 2000, 43, 2837),
(3R)-3-[(2,2,2-trifluoroacetyl)amino]-3,4-dihydro-2H-chromen-5-yl trifluoromethanesulfonate (J. Med. Chem. 2000, 43, 2837).

Other starting materials used were either available from commercial sources or prepared according to literature procedures.

### Example 1

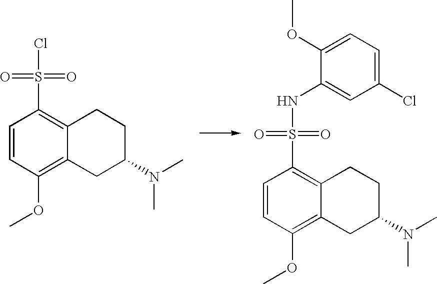

### (i) (6S)—N-(5-Chloro-2-methoxyphenyl)-6-(dimethylamino)-4-methoxy-5,6,7,8-tetrahydronaphthalene-1-sulfonamide

(6S)-6-(Dimethylamino)-4-methoxy-5,6,7,8-tetrahydronaphthalene-1-sulfonyl chloride (68 mg, 0.22 mmol), dissolved in acetonitrile (300 μl), was added to a solution of triethylamine (50 μl, 0.33 mmol) and 5-chloro-2-methoxyaniline (39 μl, 0.24 mmol) in acetonitrile (300 μl). The reaction was stirred for 2 hours and was then purified by preparative HPLC to give the title compound (24 mg, 26%) as a solid. ¹H NMR (400 MHz, CDCl₃) δ ppm 7.89 (d, 1H) 7.34 (d, 1H) 6.92 (dd, 1H) 6.70 (dd, 2H) 3.86 (s, 3H) 3.79 (s, 3H) 3.46-3.60 (m, 1H) 2.88-3.07 (m, 2H) 2.41-2.58 (m, 2H) 2.38 (s, 6H) 2.09-2.25 (m, 1H) 1.44-1.64 (m, 1H); MS m/z M+H 425.

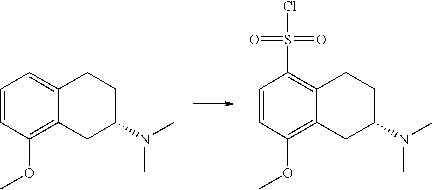

### (ii) (6S)-6-(Dimethylamino)-4-methoxy-5,6,7,8-tetrahydronaphthalene-1-sulfonyl chloride

(2S)-8-Methoxy-N,N-dimethyl-1,2,3,4-tetrahydronaphthalen-2-amine (360 mg, 1.75 mmol) was dissolved in anhydrous chloroform (5 ml) and cooled to −15° C. To the cooled solution chlorosulfonic acid (0.5 ml. 7.5 mmol) in anhydrous chloroform (5 ml) was added dropwise under 15 minutes. The reaction was stirred at −15° C. for 15 minutes and then allowed to come to room temperature for 15 minutes. The reaction mixture was added to a slurry of sodium bicarbonate (3 g)/ice and the product was extracted with chloroform (×3), dried (MgSO₄), filtered and evaporated to give the title compound (0.64 g, 87%) as a foam. The product was used without further purification; MS m/z M+H 304.

### Example 2

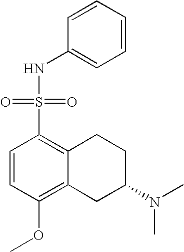

### (i) (6S)-6-(Dimethylamino)-4-methoxy-N-phenyl-5,6,7,8-tetrahydronaphthalene-1-sulfonamide

The title compound was synthesized by the analogous preparation of Example 1(i) and was isolated as an oil (18 mg, 31%). ¹H NMR (400 MHz, CDCl₃) δ ppm 7.90 (d, 1H) 7.16-7.25 (m, 2H) 6.96-7.09 (m, 3H) 6.69 (d, 1H) 3.85 (s, 3H) 3.47-3.62 (m, 1H) 2.83-3.06 (m, 2H) 2.41-2.56 (m, 2H) 2.37 (s, 6H) 2.06-2.22 (m, 1H) 1.43-1.61 (m, 1H); MS m/z M+H 361, M−H 359.

### Example 3

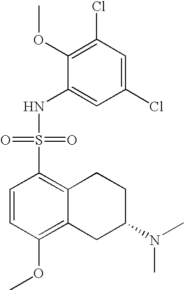

### (i) (6S)—N-(3,5-Dichloro-2-methoxyphenyl)-6-(dimethylamino)-4-methoxy-5,6,7,8-tetrahydronaphthalene-1-sulfonamide

The title compound was synthesized by the analogous preparation of Example 1(i) and was isolated as a solid (15 mg, 7%). ¹H NMR (400 MHz, CD₃OD) δ ppm 7.85 (d, 1H) 7.24 (d, 1H) 7.03 (d, 1H) 6.89 (d, 1H) 3.88 (s, 3H) 3.71 (s, 3H) 3.57-3.68 (m, 1H) 2.99-3.13 (m, 1H) 2.88-3.00 (m, 1H) 2.59-2.74 (m, 1H) 2.45-2.54 (m, 1H) 2.43 (s, 6H) 2.21 (dd, 1H) 1.44-1.66 (m, 1H); MS m/z M+H 459, M−H 457.

### Example 4

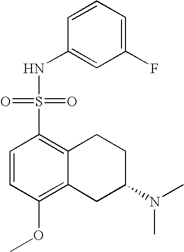

### (6S)-6-(Dimethylamino)-N-(3-fluorophenyl)-4-methoxy-5,6,7,8-tetrahydronaphthalene-1-sulfonamide

The title compound was synthesized by the analogous preparation of Example 1(i) and was isolated as an oil (28 mg, 32%). ¹H NMR (400 MHz, CD₃OD) δ ppm 7.89 (d, 1H) 7.06-7.23 (m, 1H) 6.75-6.91 (m, 3H) 6.66 (t, 1H) 3.86 (s, 3H) 3.49-3.71 (m, 1H) 3.00 (dd, 1H) 2.77-2.95 (m, 1H) 2.50-2.65 (m, 1H) 2.38-2.48 (m, 1H) 2.37 (s, 6H) 2.12-2.26 (m, 1H) 1.41-1.58 (m, 1H); MS m/z M+H 379, M−H 377.

### Example 5

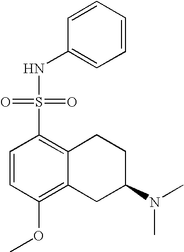

### (6R)-6-(Dimethylamino)-4-methoxy-N-phenyl-5,6,7,8-tetrahydronaphthalene-1-sulfonamide

The title compound was synthesized by the analogous preparation of Example 1(i) and was isolated as a film (19 mg, 32%). ¹H NMR (400 MHz, CDCl₃) δ ppm 7.89 (d, 1H) 7.21 (t, 2H) 6.95-7.12 (m, 3H) 6.69 (d, 1H) 3.85 (s, 3H) 3.44-3.63 (m, 1H) 2.84-3.06 (m, 2H) 2.42-2.57 (m, 1H) 2.37 (s, 6H) 2.05-2.20 (m, 1H) 1.42-1.65 (m, 1H); MS m/z M+H 361, M−H 359.

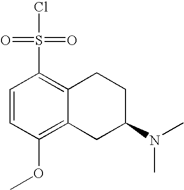

### (ii) (6R)-6-(Dimethylamino)-4-methoxy-5,6,7,8-tetrahydronaphthalene-1-sulfonyl chloride

The title compound was synthesized by the analogous preparation of Example 1 (ii) and was isolated as an oil (150 mg, 15%); MS m/z M+H 304.

### Example 6

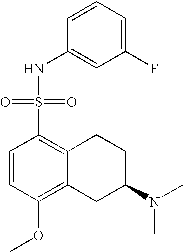

### (6R)-6-(Dimethylamino)-N-(3-fluorophenyl)-4-methoxy-5,6,7,8-tetrahydronaphthalene-1-sulfonamide

The title compound was synthesized by the analogous preparation of Example 1(i) and was isolated as an oil (18 mg, 29%). ¹H NMR (400 MHz, CDCl₃) δ ppm 7.92 (d, 1H) 7.11-7.22 (m, 1H) 6.75-6.86 (m, 3H) 6.72 (d, 1H) 3.86 (s, 3H) 3.49-3.60 (m, 1H) 2.86-3.02 (m, 2H) 2.39-2.57 (m, 2H) 2.37 (s, 6H) 2.09-2.20 (m, 1H) 1.46-1.63 (m, 1H); MS m/z M+H 379, M−H 377.

### Example 7

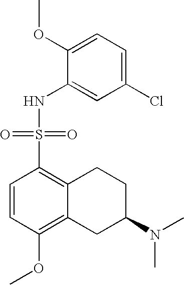

### (6R)—N-(5-Chloro-2-methoxyphenyl)-6-(dimethylamino)-4-methoxy-5,6,7,8-tetrahydronaphthalene-1-sulfonamide

The title compound was synthesized by the analogous preparation of Example 1(i) and was isolated as an oil (13 mg, 19%). ¹H NMR (400 MHz, CDCl₃) δ ppm 7.90 (d, 1H) 7.34 (d, 1H) 6.92 (dd, 1H) 6.65-6.77 (m, 2H) 3.86 (s, 3H) 3.80 (s, 3H) 3.47-3.60 (m, 1H) 2.86-3.05 (m, 2H) 2.42-2.58 (m, 2H) 2.38 (s, 6H) 2.11-2.25 (m, 1H) 1.48-1.63 (m, 1H); MS m/z M+H 425, M−H 423.

### Example 8

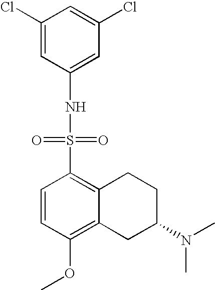

### (i) (6S)—N-(3,5-Dichlorophenyl)-6-(dimethylamino)-4-methoxy-5,6,7,8-tetrahydronaphthalene-1-sulfonamide

Sodium cyanoborohydride (48 mg, 0.76 mmol) was added portionwise to a stirred mixture of (6S)-6-amino-N-(3,5-dichlorophenyl)-4-methoxy-5,6,7,8-tetrahydronaphthalene-1-sulfonamide (110 mg, 0.252 mmol) and 37% aqueous formaldehyde solution (204 mg, 2.5 mmol) in methanol (2 ml), followed by the addition of glacial acetic acid (50 μl). The resulting mixture was stirred for 6 hours, then the solvent was evaporated, the residue was taken up in dichloromethane, washed with saturated aqueous sodium hydrogen carbonate solution, dried with sodium sulfate and concentrated. The compound was purified by preparative reversed phase HPLC to afford the title compound as a solid (71 mg, 65%). ¹H NMR (400 MHz, CDCl₃) δ ppm 7.96 (d, 1H) 7.04 (d, 2H) 6.88 (s, 1H) 6.75 (d, 1H) 3.88 (s, 3H) 3.64-3.75 (m, 1H) 3.31-3.43 (m, 1H) 3.09 (dd, 1H) 2.90-3.03 (m, 1H) 2.67 (s, 6H) 2.49-2.61 (m, 1H) 2.32-2.41 (m, 1H) 1.63-1.78 (m, 1H). MS m/z M+H 429, 431, 433, M−H 427, 429, 431.

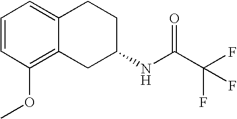

### (ii) 2,2,2-trifluoro-N-[(2S)-8-methoxy-1,2,3,4-tetrahydronaphthalen-2-yl]acetamide

Trifluoroacetic anhydride (11.4 g, 54.3 mmol) was added dropwise over a period of 20 minutes to a mixture of (2S)-8-methoxy-1,2,3,4-tetrahydronaphthalen-2-amine hydrochloride (8.29 g, 38.8 mmol) and pyridine (9.4 ml, 0.116 mol) in dichloromethane (200 ml) at ambient temperature. The resulting solution was stirred for one hour and then washed with water and 1 M aqueous sodium hydrogen carbonate solution, dried over sodium sulfate and evaporated to give the title compound as a solid (10.43 g, 98%). ¹H NMR (400 MHz, CDCl₃) δ ppm 7.15 (t, 1H) 6.75 (d, 1H) 6.70 (d, 1H) 6.41 (s, 1H) 4.27-4.37 (m, 1H) 3.83 (s, 3H) 3.17 (dd, 1H) 2.83-2.99 (m, 2H) 2.55 (dd, 1H) 2.06-2.15 (m, 1H) 1.78-1.90 (m, 1H). MS m/z M−H 272.

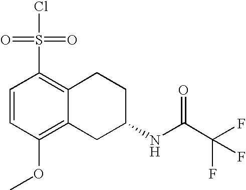

### (iii) (6S)-4-Methoxy-6-[(trifluoroacetyl)amino]-5,6,7,8-tetrahydronaphthalene-1-sulfonyl chloride

A solution of chlorosulfonic acid (9.6 g, 82 mmol) in chloroform (5 ml) was added drop-wise to a solution of 2,2,2-trifluoro-N-[(2S)-8-methoxy-1,2,3,4-tetrahydronaphthalen-2-yl]acetamide (5.42 g, 19.84 mmol) in chloroform (100 ml) at 10° C. After completion of the addition, the mixture was stirred at ambient temperature for one hour and then poured out on ice. The phases were separated and the aqueous phase was extracted with dichloromethane. The combined organic phases were washed with aqueous sodium hydrogen carbonate solution, dried over sodium sulfate and concentrated to give a solid (6.12 g, 83%) ¹H NMR (400 MHz, CDCl₃) δ ppm 8.04 (d, 1H) 6.85 (d, 1H) 6.36 (s, 1H) 3.95 (s, 3H) 3.51-3.61 (m, 1H) 3.24-3.36 (m, 2H) 2.55 (dd, 1H) 2.20-2.28 (m, 1H) 1.83-1.94 (m, 1H) 1.60 (s, 1H). MS m/z M+NH 389, 391, M−H 370, 372, M−HCl 334.

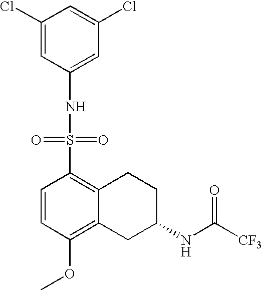

### (iv) N-((2S)-5-{[(3,5-Dichlorophenyl)amino]sulfonyl}-8-methoxy-1,2,3,4-tetrahydronaphthalen-2-yl)-2,2,2-trifluoroacetamide

Pyridine (374 μl, 4.62 mmol) was added to a suspension of (6S)-4-methoxy-6-[(trifluoroacetyl)amino]-5,6,7,8-tetrahydronaphthalene-1-sulfonyl chloride (1.145 g, 3.080 mmol) and 3,5-dichloroaniline (0.496 g, 3.080 mmol) in dichloromethane (10 ml) and the resulting mixture was stirred at ambient temperature for 16 hours. The precipitate that had formed was filtered, washed with diethyl ether and dried under vacuum to give the title compound as a solid (1.23 g, 80%). The compound was used in the subsequent step without further purification. ¹H NMR (400 MHz, CD₃OD) δ ppm 7.95 (d, 1H) 7.02 (s, 3H) 6.95 (d, 1H) 4.01-4.14 (m, 1H) 3.89 (s, 3H) 3.42-3.53 (m, 1H) 3.12 (dd, 1H) 2.97-3.08 (m, 1H) 2.53 (dd, 1H) 2.06-2.15 (m, 1H) 1.74-1.87 (m, 1H). MS m/z M+H 497, 499, 500, 501, M+NH₄ 514, 516, 517, 518, M−H 495, 497, 498, 499.

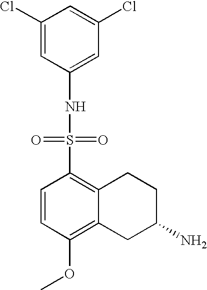

### (v) (6S)-6-Amino-N-(3,5-dichlorophenyl)-4-methoxy-5,6,7,8-tetrahydronaphthalene-1-sulfonamide

A solution of N-((2S)-5-{[(3,5-dichlorophenyl)amino]sulfonyl}-8-methoxy-1,2,3,4-tetrahydronaphthalen-2-yl)-2,2,2-trifluoroacetamide (512 mg, 1.03 mmol) in methanol (10 ml) was stirred with 2 M aqueous sodium hydroxide solution (5 ml) at ambient temperature for 6 hours and then stored at 10° C. for 16 hours. The mixture was neutralized with 1 M aqueous hydrochloric acid (8.5 ml) to pH 9. The precipitate formed was filtered, washed with water and ethyl acetate and dried to afford the title compound as the hydrochloride salt (336 mg, 75%). ¹H NMR (400 MHz, DMSO-d₆) δ ppm 7.73 (d, 1H) 6.83 (d, 1H) 6.69 (d, 2H) 6.50 (t, 1H) 3.80 (s, 3H) 3.61-3.73 (m, 1H) 3.28-3.39 (m, 1H) 3.04 (dd, 1H) 2.85-2.99 (m, 1H) 2.37 (dd, 1H) 2.01-2.10 (m, 1H) 1.48-1.62 (m, 1H). MS m/z M+H 401, 403, 404, 405, M−H 399, 401, 402, 403.

### Example 9

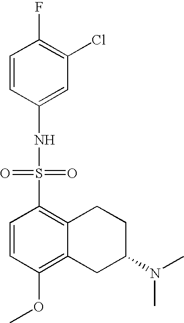

### (i) (6S)—N-(3-Chloro-4-fluorophenyl)-6-(dimethylamino)-4-methoxy-5,6,7,8-tetrahydronaphthalene-1-sulfonamide

(6S)-6-Amino-N-(3-chloro-4-fluorophenyl)-4-methoxy-5,6,7,8-tetrahydronaphthalene-1-sulfonamide (259 mg, 0.67 mmol) was transformed according to the method as described in Example 8(i) to the title compound which was obtained as a solid (156 mg, 56%). ¹H NMR (400 MHz, CDCl₃) δ ppm 7.83 (d, 1H) 7.18 (dd, 1H) 6.89-6.95 (m, 1H) 6.82 (t, 1H) 6.68 (d, 1H) 3.84 (s, 3H) 3.64-3.73 (m, 1H) 3.28-3.39 (m, 1H) 2.90-3.11 (m, 2H) 2.66 (s, 6H) 2.53 (dd, 1H) 2.27-2.41 (m, 1H) 1.60-1.76 (m, 1H). MS m/z M+H 413, 415.

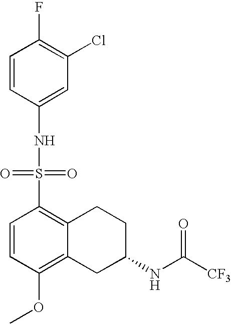

### (ii) N-((2S)-5-{[(3-Chloro-4-fluorophenyl)amino]sulfonyl}-8-methoxy-1,2,3,4-tetrahydronaphthalen-2-yl)-2,2,2-trifluoroacetamide

Pyridine (176 μl, 2.17 mmol) was added to a suspension of (6S)-4-methoxy-6-[(trifluoroacetyl)amino]-5,6,7,8-tetrahydronaphthalene-1-sulfonyl chloride (538 mg, 1.45 mmol) and 3-chloro-4-fluoroaniline (210 mg, 1.45 mmol) in dichloromethane (7 ml) and the resulting mixture was stirred at ambient temperature for 16 hours. The mixture was diluted with dichloromethane, washed with 1 M hydrochloric acid, water and saturated aqueous sodium hydrogen carbonate solution, dried with sodium sulfate and evaporated. The residue was dried under vacuum to give the title compound as a solid (539 mg, 77%), which was used without further purification. MS m/z M+H 481, 483, M+NH₄ 498, 500, M−H 479, 481.

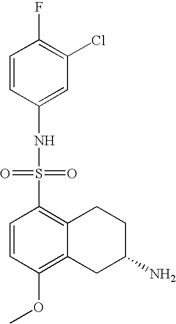

### (iii) (6S)-6-Amino-N-(3-chloro-4-fluorophenyl)-4-methoxy-5,6,7,8-tetrahydronaphthalene-1-sulfonamide

N-((2S)-5-{[(3-Chloro-4-fluorophenyl)amino]sulfonyl}-8-methoxy-1,2,3,4-tetrahydronaphthalen-2-yl)-2,2,2-trifluoroacetamide (330 mg, 686 mmol) in methanol (5 ml) was stirred together with 2 M aqueous sodium hydroxide solution (7 ml) at ambient temperature for 6 hours and then stored at 10° C. for 16 hours. The mixture was neutralized with 1 M aqueous hydrochloric acid (7 ml) and then made basic with solid sodium hydrogen carbonate. The mixture was extracted with ethyl acetate and dichloromethane, the combined extracts were dried over sodium sulfate and concentrated to give a solid (259 mg, 98%), which was used without further purification. MS m/z M+H 385, 387, M−H 383, 385.

### Example 10

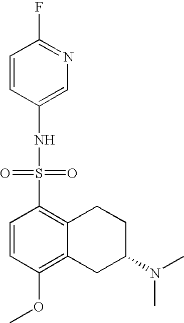

### (i) (6S)-6-(Dimethylamino)-N-(6-fluoropyridin-3-yl)-4-methoxy-5,6,7,8-tetrahydronaphthalene-1-sulfonamide

The method as described in Example 8(i) was used to convert (6S)-6-amino-N-(6-fluoropyridin-3-yl)-4-methoxy-5,6,7,8-tetrahydronaphthalene-1-sulfonamide into the title compound which was obtained as a solid (104 mg, 33%). ¹H NMR (400 MHz, CDCl₃) δ ppm 7.89 (d, 1H) 7.77 (d, 1H) 7.54-7.62 (m, 1H) 6.63-6.72 (m, 2H) 3.84 (s, 3H) 3.71 (d, 1H) 3.30-3.42 (m, 1H) 2.96-3.12 (m, 2H) 2.68 (s, 6H) 2.55 (dd, 1H) 2.32-2.40 (m, 1H) 1.70 (dd, 1H). MS m/z M+H 380, M−H 378.

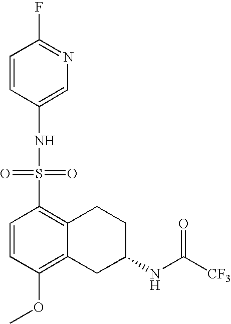

### (ii) 2,2,2-Trifluoro-N-((2S)-5-{[(6-fluoropyridin-3-yl)amino]sulfonyl}-8-methoxy-1,2,3,4-tetrahydronaphthalen-2-yl)acetamide

The method as described in Example 9(ii) was used to convert (6S)-6-amino-N-(6-fluoropyridin-3-yl)-4-methoxy-5,6,7,8-tetrahydronaphthalene-1-sulfonamide into the title compound, which was obtained as a solid (409 mg, 99%). ¹H NMR (400 MHz, DMSO-d₆) δ ppm 10.51 (br. s., 1H) 9.50 (d, 1H) 7.84-7.91 (m, 1H) 7.77 (d, 1H) 7.59-7.66 (m, 1H) 7.08 (dd, 1H) 6.94 (d, 1H) 3.95-4.12 (m, 1H) 3.83 (s, 3H) 3.35-3.45 (m, 1H) 3.17 (d, 1H) 2.92-3.05 (m, 2H) 1.95-2.05 (m, 1H) 1.67-1.80 (m, 1H). MS m/z M+H 448, M−H 446.

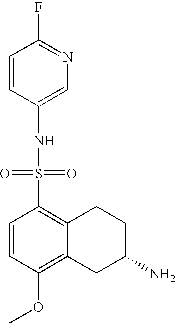

### (iii) (6S)-6-Amino-N-(6-fluoropyridin-3-yl)-4-methoxy-5,6,7,8-tetrahydronaphthalene-1-sulfonamide

The method as described in Example 9(iii) was used to convert 2,2,2-trifluoro-N-((2S)-5-{[(6-fluoropyridin-3-yl)amino]sulfonyl}-8-methoxy-1,2,3,4-tetrahydronaphthalen-2-yl)acetamide into the title compound which was obtained as a solid. MS m/z M+H 352, M−H 350.

### Example 11

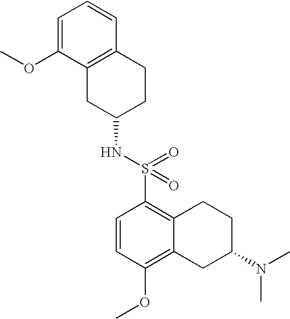

### (i) (6S)-6-(Dimethylamino)-4-methoxy-N-[(2S)-8-methoxy-1,2,3,4-tetrahydronaphthalen-2-yl]-5,6,7,8-tetrahydronaphthalene-1-sulfonamide

The method as described in Example 8(i) was used to transform (6S)-6-amino-4-methoxy-N-[(2S)-8-methoxy-1,2,3,4-tetrahydronaphthalen-2-yl]-5,6,7,8-tetrahydronaphthalene-1-sulfonamide into the title compound, which was obtained as a solid (121 mg, 60%). ¹H NMR (400 MHz, CDCl₃) δ ppm 7.94 (d, 1H) 7.06 (t, 1H) 6.74 (d, 1H) 6.65 (d, 1H) 6.60 (d, 1H) 5.01 (d, 1H) 3.88 (s, 3H) 3.73 (s, 3H) 3.54 (d, 2H) 3.08-3.20 (m, 2H) 2.90-3.02 (m, 1H) 2.72-2.90 (m, 3H) 2.62 (s, 6H) 2.51-2.59 (m, 1H) 2.46 (dd, 1H) 2.21-2.31 (m, 1H) 1.86-1.95 (m, 1H) 1.64-1.77 (m, 1H) 1.52-1.64 (m, 1H). MS m/z M+H 445, M−H 443.

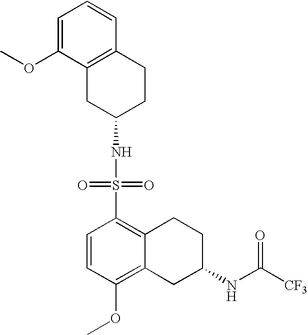

### (ii) 2,2,2-Trifluoro-N-[(2S)-8-methoxy-5-({[(2S)-8-methoxy-1,2,3,4-tetrahydronaphthalen-2-yl]amino}sulfonyl)-1,2,3,4-tetrahydronaphthalen-2-yl]acetamide

A mixture of (2S)-8-methoxy-1,2,3,4-tetrahydronaphthalen-2-amine hydrochloride (96 mg, 0.449 mmol), (6S)-4-methoxy-6-[(trifluoroacetyl)amino]-5,6,7,8-tetrahydronaphthalene-1-sulfonyl chloride (167 mg, 0.449 mmol) and DIPEA (174 mg, 1.348 mmol) in dichloromethane (10 ml) was stirred for four days at ambient temperature. The mixture was diluted with dichloromethane, washed with 1 M hydrochloric acid, water and saturated aqueous sodium hydrogen carbonate solution, and then dried over sodium sulfate and evaporated to give the title compound as a solid (165 mg, 72%), which was used in the next reaction step without further purification. MS m/z M+H 513.

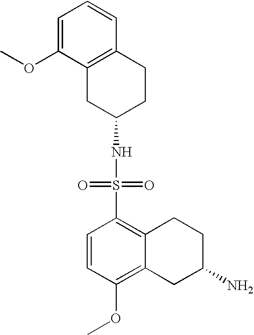

### (iii) (6S)-6-Amino-4-methoxy-N-[(2S)-8-ethoxy-1,2,3,4-tetrahydronaphthalen-2-yl]-5,6,7,8-tetrahydronaphthalene-1-sulfonamide

The method as described in Example 9(iii) was used to convert 2,2,2-trifluoro-N-[(2S)-8-methoxy-5-({[(2S)-8-methoxy-1,2,3,4-tetrahydronaphthalen-2-yl]amino}sulfonyl)-1,2,3,4-tetrahydronaphthalen-2-yl]acetamide into the title compound which was obtained as a solid (180 mg, 0.433 mmol). MS m/z M+H 417, M−H 415.

### Example 12

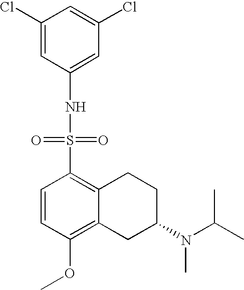

### (i) (6S)—N-(3,5-Dichlorophenyl)-6-[isopropyl(methyl)amino]-4-methoxy-5,6,7,8-tetrahydronaphthalene-1-sulfonamide

Sodium cyanoborohydride (50 mg, 0.789 mmol) was added portion-wise to a stirred mixture of (6S)-6-amino-N-(3,5-dichlorophenyl)-4-methoxy-5,6,7,8-tetrahydronaphthalene-1-sulfonamide (115 mg, 0.263 mmol) and acetone (153 mg, 2.63 mmol) in methanol (2 ml), followed by the addition of glacial acetic acid (50 μl). After 3 hours reaction time the starting material was fully converted to the monoisopropylamine derivative as evidenced by HPLC-MS (m/z M+H 443, 445, 446, 447, M−H 441, 443, 444, 445). Then 37% aqueous formaldehyde solution (79 mg, 2.63 mmol) was added followed by portion-wise addition of sodium cyanoborohydride (50 mg, 0.789 mmol) and finally addition of glacial acetic acid (50 μl). The resulting mixture was stirred for 20 hours. The methanol was evaporated and the residue was taken up in dichloromethane and washed with saturated aqueous sodium hydrogen carbonate solution, dried over sodium sulfate and evaporated. Purification by column chromatography on silica using increasingly polar mixtures of dichloromethane-methanol gave the title compound as a solid (100 mg, 83%). ¹H NMR (400 MHz, CDCl₃) δ ppm 7.92 (d, 1H) 6.97 (d, 2H) 6.94 (d, 1H) 6.72 (d, 1H) 5.52 (br. s., 1H) 3.85 (s, 3H) 3.50-3.61 (m, 1H) 3.15-3.28 (m, 1H) 2.86-3.02 (m, 3H) 2.46-2.58 (m, 1H) 2.33 (s, 3H) 2.08-2.18 (m, 1H) 1.55-1.69 (m, 1H) 1.11 (dd, 6H). MS m/z M+H 457, 459, 460, 461, M−H 455, 457, 459.

### Example 13

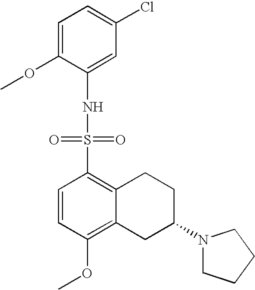

### (i) (6S)—N-(5-Chloro-2-methoxyphenyl)-4-methoxy-6-pyrrolidin-1-yl-5,6,7,8-tetrahydronaphthalene-1-sulfonamide

A mixture of (6S)-6-amino-N-(5-chloro-2-methoxyphenyl)-4-methoxy-5,6,7,8-tetrahydronaphthalene-1-sulfonamide (161 mg, 0.406 mmol), 1,4-dibromobutane (61 μl, 0.507 mmol), sodium hydrogen carbonate (215 mg, 2.03 mmol) and potassium iodide (6 mg, 0.04 mmol) in toluene was heated to reflux for 20 hours. Additional 1,4-dibromobutane (30 μl, 0.253 mmol) was added and heating was continued for 2.5 hours. The mixture was cooled to room temperature, diluted with dichloromethane, washed with saturated aqueous sodium hydrogen carbonate solution, and then dried over sodium sulfate and concentrated. Purification of the residue by preparative HPLC gave the title compound as a solid (49 mg, 27%). ¹H NMR (400 MHz, CDCl₃) δ ppm 7.90 (d, 1H) 7.33 (d, 1H) 6.93 (dd, 1H) 6.71 (d, 1H) 6.69 (d, 1H) 3.86 (s, 3H) 3.80 (s, 3H) 3.49-3.54 (m, 1H) 3.45-3.49 (m, 1H) 3.12 (d, 1H) 2.89-3.04 (m, 5H) 2.55-2.67 (m, 2H) 2.24-2.33 (m, 1H) 1.89-1.95 (m, 4H) 1.66-1.80 (m, 1H).). MS m/z M+H 451, 453, M−H 449, 450, 451.

### (ii) N-((2S)-5-{[(5-Chloro-2-methoxyphenyl)amino]sulfonyl}-8-methoxy-1,2,3,4-tetrahydronaphthalen-2-yl)-2,2,2-trifluoroacetamide

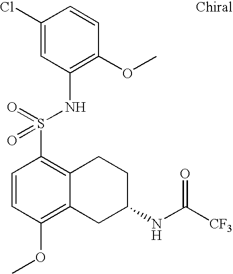

The title compound was prepared, using 2-chloro-5-methoxyaniline as the amine (1.062 g, 6.736 mmol), with the same method as described in Example 9(ii). Purification of the product by silica column chromatography using a gradient of heptane/ethyl acetate reaching from 0-100% of ethyl acetate gave the title product as a solid (2.50 g, 79%).

¹H NMR (400 MHz, CDCl₃) δ ppm 7.94 (d, 1H) 7.29 (d, 1H) 7.16 (s, 1H) 6.92 (dd, 1H) 6.72 (dd, 2H) 6.52 (d, 1H) 4.15-4.25 (m, 1H) 3.85 (s, 3H) 3.81 (s, 3H) 3.41-3.51 (m, 1H) 3.05-3.21 (m, 2H) 2.47 (dd, 1H) 2.09-2.19 (m, 1H) 1.73-1.86 (m, 1H). MS m/z M+H 493, 495, M+N 510, 512, M−H 491, 493.

### (iii) (6S)-6-Amino-N-(5-chloro-2-methoxyphenyl)-4-methoxy-5,6,7,8-tetrahydronaphthalene-1-sulfonamide

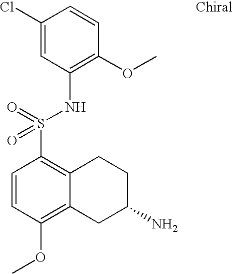

A mixture of N-((2S)-5-{[(5-chloro-2-methoxyphenyl)amino]sulfonyl}-8-methoxy-1,2,3,4-tetrahydronaphthalen-2-yl)-2,2,2-trifluoroacetamide (2.50 g, 5.07 mmol) in chloroform (50 ml) and 2 M aqueous sodium hydroxide solution (25 ml) was vigorously stirred at ambient temperature for 4.5 hours and then acidified with 5 M hydrochloric acid to pH 4. The precipitated product was filtered, washed with water and ethyl acetate and dried under vacuum to give the title compound as the hydrochloride salt (1.77 g, 81%). ¹H NMR (400 MHz, DMSO-d₆) δ ppm 7.64 (d, 1H) 7.06 (d, 1H) 6.78 (d, 1H) 6.73 (d, 1H) 6.65 (dd, 1H) 4.90 (br. s., 4H) 3.77 (s, 3H) 3.63-3.71 (m, 1H) 3.61 (s, 3H) 3.17-3.28 (m, 1H) 3.04 (dd, 1H) 2.88-3.00 (m, 1H) 2.36 (dd, 1H) 2.00-2.11 (m, 1H) 1.47-1.60 (m, 1H). MS m/z M+H 397, 399, M−H 395, 397.

### Example 14

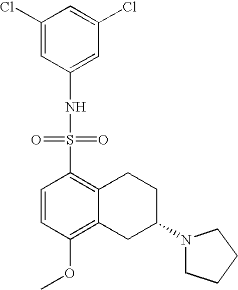

### (6S)—N-(3,5-Dichlorophenyl)-4-methoxy-6-pyrrolidin-1-yl-5,6,7,8-tetrahydronaphthalene-1-sulfonamide

A mixture of (6S)-6-amino-N-(3,5-dichlorophenyl)-4-methoxy-5,6,7,8-tetrahydronaphthalene-1-sulfonamide (61 mg, 0.14 mmol), 1,4-dibromobutane (33 μl, 0.28 mmol), DIPEA (81 μl, 0.49 mmol) and potassium iodide (2.3 mg, 0.014 mmol) in toluene (5 ml) was heated to reflux for 7 hours. The mixture was cooled to ambient temperature, diluted with ethyl acetate, washed with aqueous citric acid solution (pH 4) followed by saturated aqueous sodium hydrogen carbonate solution, dried over sodium sulfate and evaporated. Purification by reversed phase HPLC gave the title product as a solid (10 mg, 15%). ¹H NMR (400 MHz, CDCl₃) δ ppm 7.94 (d, 1H) 6.99 (d, 1H) 6.97 (d, 2H) 6.75 (d, 1H) 3.88 (s, 3H) 3.44-3.56 (m, 1H) 3.09 (d, 1H) 2.89-3.01 (m, 1H) 2.86 (br. s., 4H) 2.52-2.67 (m, 2H) 2.20-2.30 (m, 1H) 1.90 (br. s., 4H) 1.67-1.81 (m, 1H). MS m/z M+H 455, 457, 459, M−H 453, 455, 456, 457.

### Example 15

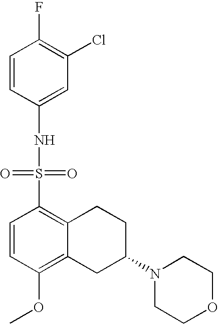

### (6S)—N-(3-Chloro-4-fluorophenyl)-4-methoxy-6-morpholin-4-yl-5,6,7,8-tetrahydronaphthalene-1-sulfonamide

A mixture of (6S)-6-amino-N-(3-chloro-4-fluorophenyl)-4-methoxy-5,6,7,8-tetrahydronaphthalene-1-sulfonamide (35 mg, 0.090 mmol), 2,2′-dibromodiethyl ether (25 mg, 0.11 mmol) and DIPEA (30 μl, 0.18 mmol) in toluene (5 ml) was heated to 80° C. for 64 hours under an atmosphere of argon. The mixture was cooled to room temperature, diluted with dichloromethane, washed with water and saturated aqueous sodium hydrogen carbonate solution, dried over sodium sulfate and evaporated. Purification by reversed phase HPLC gave the title compound as a solid (5.5 mg, 13%). ¹H NMR (400 MHz, CDCl₃) δ ppm 7.84 (d, 1H) 7.09 (dd, 1H) 6.99 (t, 1H) 6.85-6.91 (m, 1H) 6.72 (d, 1H) 3.88 (s, 3H) 3.77 (t, 4H) 3.50 (dt, 1H) 3.00 (dd, 1H) 2.86-2.96 (m, 1H) 2.63-2.72 (m, 4H) 2.56-2.63 (m, 1H) 2.47-2.57 (m, 1H) 2.12-2.21 (m, 1H) 1.51-1.68 (m, 2H). MS m/z M+H 455, 457, M−H 453, 455.

### Example 16

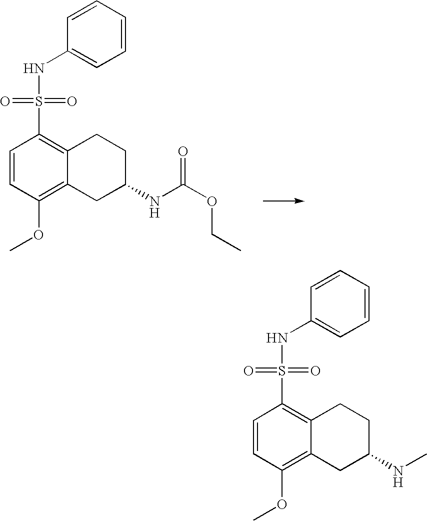

### (i) (6S)-4-Methoxy-6-(methylamino)-N-phenyl-5,6,7,8-tetrahydronaphthalene-1-sulfonamide

To a suspension of lithium aluminum hydride (39 mg, 1 mmol) in anhydrous THF (1 ml) was ethyl [(2S)-5-(anilinosulfonyl)-8-methoxy-1,2,3,4-tetrahydronaphthalen-2-yl]carbamate (140 mg, 0.35 mmol), in THF (2 ml), added dropwise. The reaction stirred for 1 hour at room temperature and was then refluxed for 10 minutes. The reaction was carefully quenched with saturated aqueous sodium sulfate (200 μl), the reaction mixture was filtered, washed with THF and the solvent was evaporated. The remains were purified by preparative HPLC to give the title compound as an oil (64 mg, 53%). ¹H NMR (400 MHz, CD₃OD) δ ppm 7.85 (d, 1H) 7.16 (t, 2H) 7.03 (d, 2H) 6.96 (t, 1H) 6.83 (d, 1H) 3.85 (s, 3H) 3.45-3.55 (m, 1H) 3.09 (dd, 1H) 2.86-3.00 (m, 1H) 2.66-2.76 (m, 1H) 2.45 (s, 3H) 2.25 (dd, 1H) 2.10-2.19 (m, 1H) 1.39-1.52 (m, 1H); MS m/z M+H 347, M−H 345.

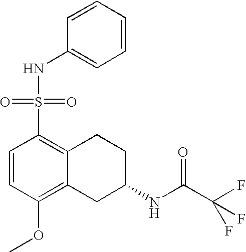

### (ii) N-[(2S)-5-(Anilinosulfonyl)-8-methoxy-1,2,3,4-tetrahydronaphthalen-2-yl]-2,2,2-trifluoroacetamide

The title compound was synthesized from (6S)-6-amino-4-methoxy-N-phenyl-5,6,7,8-tetrahydronaphthalene-1-sulfonamide by the analogous preparation of Example 8(iv) and was obtained as a solid (306 mg, 88%). ¹H NMR (400 MHz, CD₃OD) δ ppm 7.88 (d, 1H) 7.17 (t, 2H) 7.05 (d, 2H) 6.97 (t, 1H) 6.86 (d, 1H) 3.99-4.11 (m, 1H) 3.86 (s, 3H) 3.46-3.58 (m, 1H) 2.96-3.15 (m, 2H) 2.49 (dd, 1H) 2.03-2.14 (m, 1H) 1.69-1.85 (m, 1H); MS m/z M+H 429, M−H 427.

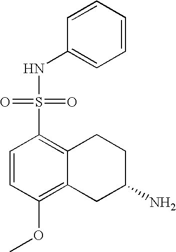

### (iii) (6S)-6-Amino-4-methoxy-N-phenyl-5,6,7,8-tetrahydroizaphthalene-1-sulfonamide

The title compound was synthesized from N-[(2S)-5-(anilinosulfonyl)-8-methoxy-1,2,3,4-tetrahydronaphthalen-2-yl]-2,2,2-trifluoroacetamide by the analogous preparation of Example 8(v) as a white solid (230 mg, 98%). ¹H NMR (400 MHz, DMSO-d₆) δ ppm 7.78 (d, 1H) 7.17 (t, 2H) 7.03 (d, 2H) 6.86-6.97 (m, 2H) 3.81 (s, 3H) 3.35-3.45 (m, 1H) 3.31 (s, 2H under H₂O peak) 2.80-3.02 (m, 3H) 2.16 (dd, 1H) 1.89 (d, 1H) 1.32-1.47 (m, 1H); MS m/z M+H 333, M−H 331.

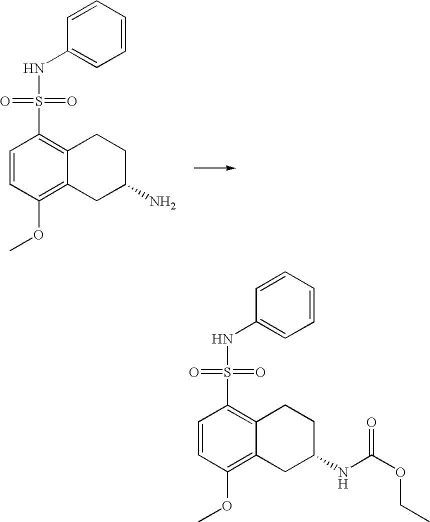

### (iv) Ethyl [(2S)-5-(Anilinosulfonyl)-8-methoxy-1,2,3,4-tetrahydronaphthalen-2-yl]carbamate

(6S)-6-Amino-4-methoxy-N-phenyl-5,6,7,8-tetrahydronaphthalene-1-sulfonamide (220 mg, 0.66 mmol) was suspended in anhydrous dichloromethane (5 ml), triethylamine (165 μl. 1.2 mmol) and ethyl chloroformate (65 μl, 0.73 mmol) was added. The reaction mixture stirred for 10 minutes at room temperature. The solvent was evaporated, ethyl acetate and a sodium bicarbonate solution were added. The mixture was extracted with ethyl acetate (×2), dried (MgSO₄), filtered and evaporated. The remains were purified on silica (45% ethyl acetate/hexane) to give the title compound as a foam (140 mg, 52%). ¹H NMR (400 MHz, CDCl₃) δ ppm 7.92 (d, 1H) 7.20 (t, 2H) 7.07 (d, 1H) 6.99-7.05 (m, 2H) 6.69 (d, 1H) 4.70 (d, 1H) 4.06-4.21 (m, 2H) 3.88-3.97 (m, 1H) 3.83 (s, 3H) 3.37-3.51 (m, 1H) 3.04-3.16 (m, 2H) 2.41 (dd, 1H) 2.05-2.11 (m, 1H) 1.64-1.77 (m, 1H) 1.20-1.32 (m, 3H); MS m/z M+H 405, M−H 403.

### Example 17

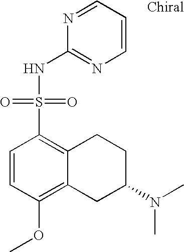

### (i) (6S)-6-(Dimethylamino)-4-methoxy-N-pyrimidin-2-yl-5,6,7,8-tetrahydronaphthalene-1-sulfonamide

(6S)-6-Amino-4-methoxy-N-pyrimidin-2-yl-5,6,7,8-tetrahydronaphthalene-1-sulfonamide (148 mg, 0.44 mmol) and formaldehyde (37% aqueous solution, 360 μL, 4.4 mmol) were mixed in methanol (5 ml) and was stirred at ambient temperature for 1 hour. Acetic acid (53 μl) and sodium cyanoborohydride (84 mg, 1.32 mmol) was added and the reaction was stirred at ambient temperature for 16 hours. The solvents were evaporated and the solid was redissolved in water and dichloromethane. The phases were separated and the water phase was washed with ethyl acetate (×3). The water phase was evaporated to dryness, acetone was added and the mixture was filtered. The acetone was evaporated and the residue was purified by preparative HPLC to afford the title compound (2.2 mg, 1.3%), ¹H NMR (400 MHz, Acetone-d6) δ ppm 8.51-8.56 (m, 2H) 8.21 (d, 1H) 7.08-7.11 (m, 1H) 7.04-7.08 (m, 1H) 4.02 (s, 3H) 3.69-3.79 (m, 1H) 2.97-3.13 (m, 2H) 2.51-2.70 (m, 2H) 2.42 (s, 6H) 2.16-2.23 (m, 1H) 1.54-1.68 (m, 1H). MS m/z M+H 363.

### (ii) N-[(2S)-5-(aminosulfonyl)-8-methoxy-1,2,3,4-tetrahydronaphthalen-2-yl]-2,2,2-trifluoroacetamide

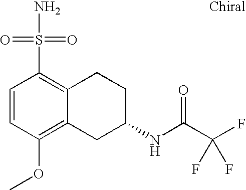

(6S)-4-Methoxy-6-[(trifluoroacetyl)amino]-5,6,7,8-tetrahydronaphthalene-1-sulfonyl chloride (60 mg, 0.16 mmol) was dissolved in methanol (1.5 ml) and ammonia (7 M in methanol, 60 μl, 0.32 mmol). The mixture was stirred at ambient temperature for 16 hours. The precipitate was collected by filtration and washed with water to give the title compound as a solid (45 mg, 80%). MS m/z M−H 351.

### (iii) 2,2,2-Trifluoro-N-{(2S)-8-methoxy-5-[(pyrimidin-2-ylamino)sulfonyl]-1,2,3,4-tetrahydronaphthalen-2-yl}acetamide

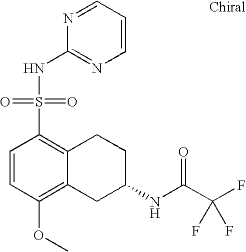

N-[(2S)-5-(Aminosulfonyl)-8-methoxy-1,2,3,4-tetrahydronaphthalen-2-yl]-2,2,2-trifluoroacetamide (88 mg, 0.25 mmol) was dissolved in DMF (3 ml) and sodium hydride (7.2 mg, 0.30 mmol) was added and the mixture was put under argon atmosphere. When the gas evolution had stopped, DIPEA (180 μl, 1.0 mmol) and 2-chloropyrimidine (98 mg, 0.85 mmol) was added and the mixture was heated at 100° C. for 6 hours by microwave irradiation. The solvent was evaporated and the crude was used without further purification. MS m/z M+H 430.

### (iv) (6S)-6-amino-4-methoxy-N-pyrimidin-2-yl-5,6,7,8-tetrahydronaphthalene-1-sulfonamide

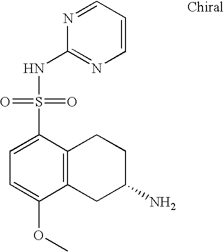

Crude 2,2,2-trifluoro-N-{(2S)-8-methoxy-5-[(pyrimidin-2-ylamino)sulfonyl]-1,2,3,4-tetrahydronaphthalen-2-yl}acetamide (from previous section) was dissolved in methanol (4 ml) and aqueous sodium hydroxide (1 M, 1.5 ml) was added. The mixture was stirred at ambient temperature for 4 hours. Hydrochloric acid (2M) was added to pH 7 and the solvents were evaporated. The solid was treated with acetone (3×30 ml) followed by methanol (5 ml). The organic solutions were combined and the solvent was removed to give a crude product (148 mg) that was used in the next step without further purification. MS m/z M+H 335.

### Example 18

### (i) (6S)-6-(Dimethylamino)-4-methoxy-N-pyridin-2-yl-5,6,7,8-tetrahydronaphthalene-1-sulfonamide

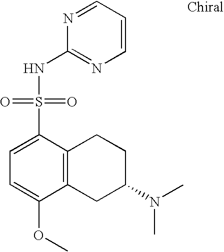

(6S)-6-Amino-4-methoxy-N-pyridin-2-yl-5,6,7,8-tetrahydronaphthalene-1-sulfonamide (166 mg, 0.5 mmol) was dissolved in methanol (4 ml), formaldehyde (aqueous solution 37%, 410 μl, 5 mmol) was added and the mixture was stirred at ambient temperature for 1 hour. Acetic acid (60 μl) was added followed by portionwise addition of sodium borohydride. The mixture was stirred at ambient temperature for 2 hours. The solvent was evaporated and ethyl acetate and dichloromethane (1:1) was added followed by saturated sodium hydrogen carbonate solution. The phases were separated and the aqueous phase was extracted with dichloromethane. The combined organic phases were dried (Na₂SO₄), filtered and the solvents were evaporated. The product was isolated by column chromatography (25 mg, 14%). ¹H NMR (400 MHz, DMSO-d₆) δ ppm 7.94-7.99 (m, 1H) 7.83-7.87 (m, 1H) 7.63-7.69 (m, 1H) 7.06 (d, 1H) 6.90 (d, 1H) 6.78-6.84 (m, 1H) 3.83 (s, 3H) 3.47-3.58 (m, 1H) 2.83-2.92 (m, 1H) 2.74-2.83 (m, 1H) 2.39-2.45 (m, 1H) 2.29-2.39 (m, 1H) 2.23 (s, 6H) 1.93-2.02 (m, 1H) 1.36-1.51 (m, 1H). MS m/z M+H 362, M−H 360

### (ii) 2,2,2-Trifluoro-N-{(2S)-8-methoxy-5-[(pyridin-2-ylamino)sulfonyl]-1,2,3,4-tetrahydronaphthalen-2-yl}acetamide

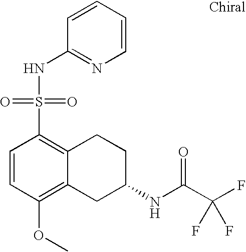

(6S)-4-Methoxy-6-[(trifluoroacetyl)amino]-5,6,7,8-tetrahydronaphthalene-1-sulfonyl chloride (186 mg, 0.50 mmol) and pyridin-2-amine (52 mg, 0.55 mmol) was dissolved in dichloromethane (5 ml). Pyridine (80 μl, 1 mmol) was added and the mixture was stirred at ambient temperature for 16 hours. The solvents were evaporated and the crude was used in the nest step without further purification. MS m/z M+H 430, M−H 428.

### (iii) (6S)-6-Amino-4-methoxy-N-pyridin-2-yl-5,6,7,8-tetrahydronaphthalene-1-sulfonamide

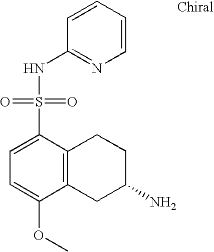

Crude 2,2,2-trifluoro-N-{(2S)-8-methoxy-5-[(pyridin-2-ylamino)sulfonyl]-1,2,3,4-tetrahydronaphthalen-2-yl}acetamide (from previous section) was dissolved in methanol (5 ml) and aqueous sodium hydroxide solution (1 M, 5 ml) was added. The mixture was stirred at ambient temperature for 2 hours. The pH was adjusted to 7 by addition of hydrochloric acid (2M) and the solvents were evaporated. The crude was used in the next step without further purification. MS m/z M+H 334 M−H 332

### Example 19

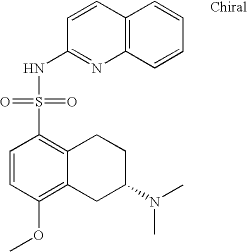

### (i) (6S)-6-(Dimethylamino)-4-methoxy-N-quinolin-2-yl-5,6,7,8-tetrahydronaphthalene-1-sulfonamide

To a solution of 2,2,2-trifluoro-N-{(2S)-8-methoxy-5-[(quinolin-2-ylamino)sulfonyl]-1,2,3,4-tetrahydronaphthalen-2-yl}acetamide (120 mg, 0.25 mmol) in methanol (5 ml) was added aqueous sodium hydroxide (1 M, 5 ml) and the mixture was stirred at ambient temperature for 2 hours. The pH was adjusted to 7 by addition of hydrochloric acid (2 M). Formaldehyde (37% aqueous solution, 0.22 ml, 2.7 mmol) and methanol (3 ml) was added and the mixture was stirred for 1 hour. Sodium cyanoborohydride (52 mg, 0.83 mmol) was added followed by acetic acid (30 μl) and the reaction mixture was stirred at ambient temperature for 16 hours. The solvents were evaporated and the residue was purified by preparative HPLC to give the title compound (5.7 mg, 3% over 2 steps). ¹H NMR (400 MHz, CD₃OD) δ ppm 8.04-8.08 (m, 1H) 7.98-8.03 (m, 1H) 7.72-7.76 (m, 1H) 7.62-7.67 (m, 1H) 7.52-7.57 (m, 1H) 7.40-7.44 (m, 1H) 7.34-7.39 (m, 1H) 6.86-6.91 (m, 1H) 3.88 (s, 3H) 3.73-3.81 (m, 1H) 2.98-3.07 (m, 2H) 2.50-2.62 (m, 1H) 2.38-2.46 (m, 1H) 2.36 (s, 6H) 2.13-2.21 (m, 1H) 1.42-1.56 (m, 1H). MS m/z M+H 412, M−H 410.

### (ii) 2,2,2-Trifluoro-N-{(2S)-8-methoxy-5-[(quinolin-2-ylamino)sulfonyl]-1,2,3,4-tetrahydronaphthalen-2-yl}acetamide

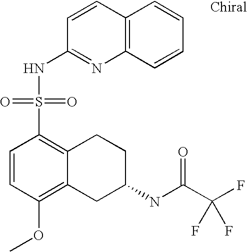

Quinolin-2-amine (79 mg, 0.55 mmol) was dissolved in dichloromethane (2.5 ml) and a solution of 2,2,2-trifluoro-N-[(2S)-8-methoxy-1,2,3,4-tetrahydronaphthalen-2-yl]acetamide in dichloromethane (2.5 ml) was added slowly. The mixture was heated by microwave irradiation at 100° C. for 3 hours. The solvent was evaporated and the crude product was used in the next step. MS m/z M+H 480.

### Example 20

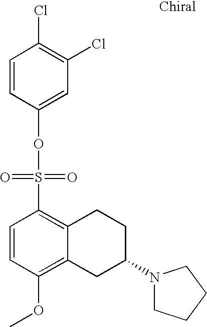

### (i) 4-Methoxy-6-pyrrolidin-1-yl-5,6,7,8-tetrahydro-naphthalene-1-sulfonic acid 3,4-dichloro-phenyl ester

A mixture of 4-methoxy-6-(2,2,2-trifluoro-acetylamino)-5,6,7,8-tetrahydro-naphthalene-1-sulfonic acid 3,4-dichloro-phenyl ester (190 mg, 0.381 mmol) and LiOH×H₂O (80 mg, 1.91 mmol) in acetonitrile (1.5 ml) was irradiated by microwaves at 100° C. for 90 min. 1,4-Dibromobutane was added and the resulting mixture was irradiated by microwaves at 120° C. for 5 min. The solvent was evaporated and the residue was purified by preparative HPLC and then by passing the product as a solution in dichloromethane through a column of silica bound amine to give the title product as a solid (52 mg, 30%). ¹H NMR (400 MHz, CDCl₃) δ ppm 7.70 (d, 1H) 7.34 (d, 1H) 7.18 (m, 1H) 6.82 (dd, 1H) 6.71 (d, 1H) 3.90 (s, 3H) 3.53-3.65 (m, 1H) 3.05-3.23 (m, 3H) 2.56-3.05 (m, 5H) 2.27-2.36 (m, 1H) 1.86-2.08 (m, 5H). MS m/z M+H 456, 458.

### (ii) 4-Methoxy-6-(2,2,2-trifluoro-acetylamino)-5,6,7,8-tetrahydro-naphthalene-1-sulfonic acid 3,4-dichloro-phenyl ester

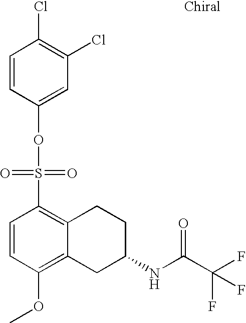

3,4-Dichlorophenol (98 mg, 0.603 mmol) and pyridine (98 μL, 1.206 mmol) were added to a solution of 4-methoxy-6-(2,2,2-trifluoro-acetylamino)-5,6,7,8-tetrahydro-naphthalene-1-sulfonyl chloride (224 mg, 0.603 mmol) in dichloromethane (2 ml). The resulting mixture was stirred at room temperature for 60 hours and then diluted with dichloromethane, washed with 1 M hydrochloric acid, water, and saturated aqueous bicarbonate. The solution was dried over Na₂SO₄ and evaporated to give an oil (200 mg, 67%). MS m/z M+NH₄ 515, 517, M−H 496, 498, 500.

### Example 21

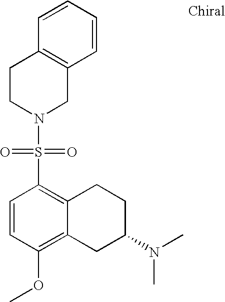

### (i) [5-(3,4-Dihydro-1H-isoquinoline-2-sulfonyl)-8-methoxy-1,2,3,4-tetrahydro-naphthalen-2-yl]-dimethyl-amine

5-(3,4-Dihydro-1H-isoquinoline-2-sulfonyl)-8-methoxy-1,2,3,4-tetrahydro-naphthalen-2-ylamine was reacted according to the method described in Example 8(i). After purification by preparative HPLC the title compound was obtained as a solid (41 mg, 36%). ¹H NMR (400 MHz, CDCl₃) δ ppm 7.97 (d, 1H) 7.15-7.21 (m, 2H) 7.10-7.15 (m, 1H) 7.01-7.08 (m, 1H) 6.79 (d, 1H) 4.36 (s, 2H) 3.90 (s, 3H) 3.43-3.65 (m, 4H) 3.13-3.22 (m, 1H) 2.95-3.06 (m, 1H) 2.83-2.95 (m, 3H) 2.62 (s, 6H) 2.21-2.33 (m, 1H) 1.57-1.74 (m, 1H). MS m/z M+H 401.

### (ii) N-[(2S)-5-(3,4-dihydroisoquinolin-2(1H)-ylsulfonyl)-8-methoxy-1,2,3,4-tetrahydronaphthalen-2-yl]-2,2,2-trifluoroacetamide

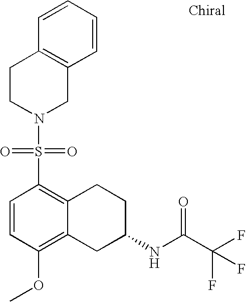

The title compound was prepared by the method described in Example 9(ii) using 1,2,3,4-tetrahydro-isoquinoline. The product was purified by chromatography on a silica column, using gradient elution by stepwise increasing amounts of ethyl acetate in heptane, starting with 10% and ending at 50% affording the title product as a white solid (135 mg, 70%). ¹H NMR (400 MHz, DMSO-d₆) δ ppm 9.50 (d, 1H) 7.85 (d, 1H) 7.10-7.24 (m, 4H) 7.04 (d, 1H) 4.32 (s, 2H) 3.95-4.07 (m, 1H) 3.86-3.91 (m, 3H) 3.39-3.47 (m, 2H) 2.86-3.07 (m, 2H) 2.79-2.87 (m, 2H) 1.90-2.00 (m, 1H) 1.60-1.76 (m, 1H) 1.23-1.31 (m, 1H). MS m/z M+H 469, M−H 467.

### (iii) 5-(3,4-Dihydro-1H-isoquinoline-2-sulfonyl)-8-methoxy-1,2,3,4-tetrahydro-naphthalen-2-ylamine

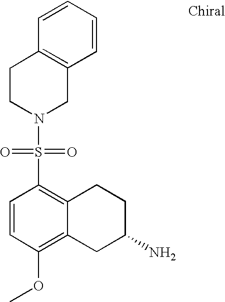

N-[5-(3,4-Dihydro-1H-isoquinoline-2-sulfonyl)-8-methoxy-1,2,3,4-tetrahydro-naphthalen-2-yl]-2,2,2-trifluoro-acetamide (132 mg, 0.282 mmol) was stirred together with 2 M NaOH (aq) in methanol-dichloromethane (1:1, 4 ml) at room temperature for 20 hours. The pH was adjusted to about 8 by the addition of 1 M hydrochloric acid and saturated aqueous bicarbonate, and then the mixture was extracted dichloromethane (×5). The combined extracts were dried over sodiumsulfate and evaporated to give the title product as an oil (116 mg, 100%). MS m/z M+H 373.

### Example 22

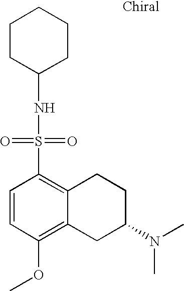

### (i) (6S)—N-cyclohexyl-6-(dimethylamino)-4-methoxy-5,6,7,8-tetrahydronaphthalene-1-sulfonamide

The title compound was prepared using the method described in Example 8 (i) and the product was obtained as a solid (48 mg, 68%).

¹H NMR (400 MHz, CDCl₃) δ ppm 7.91 (d, 1H) 6.73 (d, 1H) 4.36 (d, 1H) 3.89 (s, 3H) 3.48-3.58 (m, 1H) 3.00-3.14 (m, 2H) 2.85-2.99 (m, 1H) 2.55-2.66 (m, 1H) 2.44-2.54 (m, 1H) 2.42 (s, 6H) 2.13-2.22 (m, 1H) 1.69-1.84 (m, 2H) 1.47-1.69 (m, 4H) 1.06-1.31 (m, 4H). MS m/z M+H 367, M−H 365.

### (ii) N-(5-Cyclohexylsulfamoyl-8-methoxy-1,2,3,4-tetrahydro-naphthalen-2-yl)-2,2,2-trifluoro-acetamide

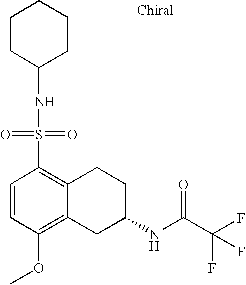

The title compound was prepared using the method described in Example 9 (ii) and the product was obtained as a solid (95 mg, 54%). MS m/z M+H 435, M−H 433.

### (iii) 6-Amino-4-methoxy-5,6,7,8-tetrahydro-naphthalene-1-sulfonic acid cyclohexylamide

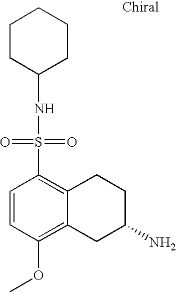

N-(5-Cyclohexylsulfamoyl-8-methoxy-1,2,3,4-tetrahydro-naphthalen-2-yl)-2,2,2-trifluoro-acetamide (93 mg, 0.21 mmol) was stirred together with 2 M aqueous NaOH (1 ml) in methanol (2 ml) at room temperature for 20 hours. The pH was adjusted to about 8 by the addition of 1 M hydrochloric acid and saturated aqueous bicarbonate, and then the mixture was extracted with five portions of dichloromethane. The combined extracts were dried over sodium sulfate and the solvent was evaporated to give the title product as a solid (65 mg, 90%). MS m/z M+H 339, M−H 337.

### Example 23

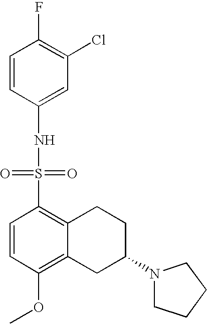

### (6S)—N-(3-Chloro-4-fluorophenyl)-4-methoxy-6-pyrrolidin-1-yl-5,6,7,8-tetrahydronaphthalene-1-sulfonamide

A mixture of (6S)-6-amino-N-(3-chloro-4-fluorophenyl)-4-methoxy-5,6,7,8-tetrahydronaphthalene-1-sulfonamide (53 mg, 0.138 mmol), 1,4-dibromobutane (36 mg, 0.165 mmol), DIPEA (50 μl, 0.304 mmol) and potassium iodide (2 mg, 0.014 mmol) in toluene (5 ml) was refluxed for 65 hours under an atmosphere of argon. The mixture was cooled to room temperature, diluted with dichloromethane, washed with water and saturated aqueous sodium hydrogen carbonate solution, dried over sodium sulfate and evaporated. Purification by reversed phase HPLC gave the title compound as a solid (46 mg, 76%). ¹H NMR (400 MHz, CDCl₃) δ ppm 7.84 (d, 1H) 7.19 (dd, 1H) 6.91-6.97 (m, 1H) 6.89 (t, 1H) 6.69 (d, 1H) 3.85 (s, 3H) 3.55-3.66 (m, 1H) 3.08-3.22 (m, 5H) 2.94-3.08 (m, 2H) 2.69 (dd, 1H) 2.28-2.39 (m, 1H) 1.98-2.02 (m, 4H) 1.80-1.95 (m, 1H). MS m/z M+H 439, 441, M−H 437, 439.

### Example 24

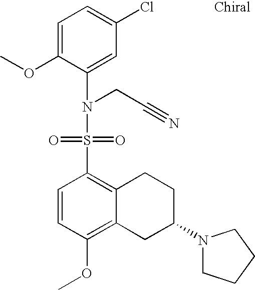

### (i) (6S)—N-(5-chloro-2-methoxyphenyl)-N-(cyanomethyl)-4-methoxy-6-pyrrolidin-1-yl-5,6,7,8-tetrahydroizaphthalene-1-sulfonamide

A solution of (6S)—N-(5-chloro-2-methoxyphenyl)-4-methoxy-6-pyrrolidin-1-yl-5,6,7,8-tetrahydronaphthalene-1-sulfonamide (250 mg, 0.554 mmol) in anhydrous THF (3 ml) was added to a suspension of NaH (18 mg, 0.69 mmol) in anhydrous THF (4 ml). The mixture was stirred at ambient temperature for 10 minutes and then cooled to −50° C. Bromoacetonitrile (83 mg, 069 mmol) was added and the mixture was warmed to room temperature and stirred for 18 hours. Anhydrous DMF (1 ml) was added, a second portion of NaH (18 mg, 0.69 mmol) was added and the mixture was stirred at room temperature for 2 hours, heated with microwave irradiation to 100° C. for 5 min and then to 120° C. for 20 min. The solvents were evaporated and the residue was taken up in dichloromethane, washed with water and dried over Na₂SO₄. Evaporation and purification by flash chromatography using gradient elution (0-15% methanol in dichloromethane) gave a mixture containing the starting material and the title compound. The mixture was dissolved in anhydrous DMF (2 ml) and anhydrous K₂CO₃ (40 mg, 0.29 mmol) and bromoacetonitrile (52 mg, 0.43 mmol) were added to the solution. The resulting mixture was heated by microwave irradiation at 140° C. for 10 min. The mixture was filtered, the solvent was evaporated and the residue was purified by preparative HPLC to afford the title compound as a solid (20 mg, 7%). ¹H NMR (400 MHz, CDCl₃) δ ppm 7.74 (d, 1H) 7.45 (d, 1H) 7.29 (dd, 1H) 6.79 (d, 1H) 6.68 (d, 1H) 4.54 (dd, 2H) 3.87 (s, 3H) 3.63 (s, 3H) 3.10-3.29 (m, 3H) 2.48-3.01 (m, 5H) 2.15-2.26 (m, 1H) 1.92-2.11 (m, 6H). MS m/z M+H 490, 492.

### Example 25

### (i) (6S)—N-(4-chlorophenyl)-4-methoxy-6-(methylamino)-5,6,7,8-tetrahydronaphthalene-1-sulfonamide

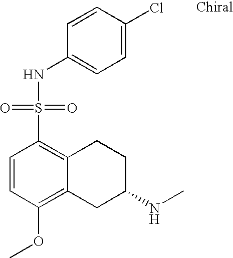

A solution of ethyl ((2S)-5-{[(4-chlorophenyl)amino]sulfonyl}-8-methoxy-1,2,3,4-tetrahydronaphthalen-2-yl)carbamate (133 mg, 0.30 mmol) in THF (3.5 ml) was added dropwise to a suspension of lithium aluminum hydride (36 mg, 0.91 mmol) in THF (1 ml). The mixture was stirred at ambient temperature for 2 hours and was heated at reflux for 1 hour. The reaction was quenched by dropwise addition of saturated aqueous Na₂SO₄ (400 μl). The mixture was filtrated and the solvent was evaporated. The product was isolated by preparative HPLC to give a solid (55 mg, 48%). ¹H NMR (400 MHz, CDCl₃) δ ppm 7.89 (d, 1H) 7.00-7.09 (m, 4H) 6.70 (d, 1H) 3.85 (s, 3H) 3.63-3.72 (m, 1H) 3.30-3.39 (m, 1H) 3.22-3.30 (m, 1H) 2.94-3.09 (m, 2H) 2.77 (s, 3H) 2.58 (dd, 1H) 2.37-2.45 (m, 1H). MS m/z M+H 381, 383, M−H 379, 381.

### (ii) N-((2S)-5-{[(4-Chlorophenyl)amino]sulfonyl}-8-methoxy-1,2,3,4-tetrahydronaphthalen-2-yl)-2,2,2-trifluoroacetamide

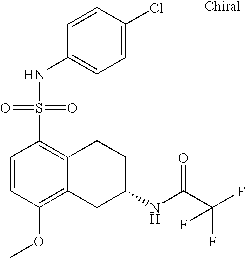

The title compound was prepared using the method described in Example 9 (ii) and the product was obtained as a solid (240 mg, 96%). MS m/z M+H 463, 465, M−H 461, 463.

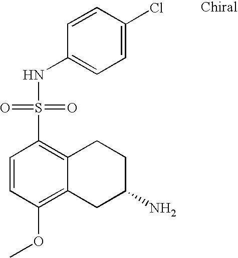

### (iii) (6S)-6-Amino-N-(4-chlorophenyl)-4-methoxy-5,6,7,8-tetrahydronaphthalene-1-sulfonamide

The title compound was prepared using the method described in Example 9 (iii) and the product was obtained as a solid (180 mg, 95%). MS m/z M+H 367, 369, M−H 365, 367.

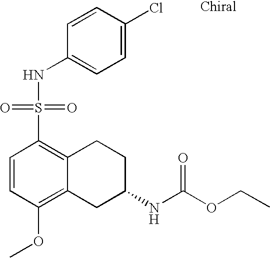

### (iv) Ethyl ((2S)-5-{[(4-chlorophenyl)amino]sulfonyl}-8-methoxy-1,2,3,4-tetrahydronaphthalen-2-yl)carbamate

(6S)-6-Amino-N-(4-chlorophenyl)-4-methoxy-5,6,7,8-tetrahydronaphthalene-1-sulfonamide (180 mg, 0.49 mmol) was dissolved in dichloromethane (5 ml) and ethylchloroformate (47 μl, 0.49 mmol) and triethylamine (171 μl, 1.203 mmol) were added. The mixture was stirred at ambient temperature for 20 min. Dichloromethane (25 ml) was added and the mixture was washed with 1M hydrochloric acid followed by saturated sodium hydrogen carbonate solution. The organic phase was dried (Na₂SO₄) and is the solvent was evaporated. The residue was purified by chromatography on silica using a gradient of heptane/ethyl acetate reaching from 0-100% of ethyl acetate to give a solid (133 mg, 62%). ¹H NMR (400 MHz, CDCl₃) δ ppm 7.89 (d, 1H) 7.18 (d, 2H) 6.96 (d, 2H) 6.71 (d, 1H) 4.62-4.71 (m, 1H) 4.10-4.19 (m, 2H) 3.89-3.99 (m, 1H) 3.85 (s, 3H) 3.37-3.48 (m, 1H) 3.02-3.16 (m, 2H) 2.42 (dd, 1H) 2.05-2.14 (m, 1H) 1.68-1.80 (m, 1H) 1.27 (t, 3H). MS m/z M+H 439, 441, M−H 437, 439.

### Example 26

### (i) (6S)-4-methoxy-6-pyrrolidin-1-yl-N-[3-(trifluoromethyl)phenyl]-5,6,7,8-tetrahydronaphthalene-1-sulfonamide

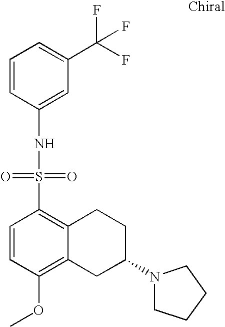

(6S)-6-amino-4-methoxy-N-[3-(trifluoromethyl)phenyl]-5,6,7,8-tetrahydronaphthalene-1-sulfonamide (138 mg, 0.35 mmol), 1,4-dibromobutane (112 mg, 0.52 mmol), DIPEA (0.295 ml, 1.72 mmol) and potassium iodide (14 mg, 0.09 mmol) in toluene (2.5 ml) were heated to reflux for 20 hours. Dichloromethane (20 ml) was added and the organic phase was washed with citric acid (pH 4), water and dried over sodium sulfate. The solvent was removed under reduced pressure and the residue was purified by HPLC to give the title compound as a solid (10.2 mg, 15%).

¹H NMR (400 MHz, CDCl₃) δ ppm 7.90 (d, 1H) 7.33-7.36 (m, 1H) 7.18-7.26 (m, 3H) 6.68 (d, 1H) 3.83 (s, 3H) 3.47-3.57 (m, 1H) 3.00-3.09 (m, 1H) 2.86-2.97 (m, 1H) 2.67-2.76 (m, 4H) 2.32-2.53 (m, 2H) 2.16-2.25 (m, 1H) 1.79-1.87 (m, 4H) 1.57-1.69 (m, 1H). MS m/z M+H 455, M−H 453.

### (ii) (6S)-6-amino-4-methoxy-N-[3-(trifluoromethyl)phenyl]-5,6,7,8-tetrahydronaphthalene-1-sulfonamide

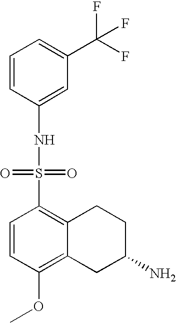

2,2,2-trifluoro-N-[(2S)-8-methoxy-5-({[3-(trifluoromethyl)phenyl]amino}sulfonyl)-1,2,3,4-tetrahydronaphthalen-2-yl]acetamide (279 mg, 0.562 mmol) were dissolved in methanol (2.5 ml). Aqueous sodium hydroxide solution (2 M, 1.5 ml) were added and the reaction mixture was stirred for 16 hours at RT. The mixture was made neutral by addition of hydrochloric acid (1 M) and the mixture was extracted with dichloromethane. The organic phase was dried (Na₂SO₄) and the solvent was evaporated to give the title compound (228 mg, 99%), which was used without further purification in the next reaction steps.

MS m/z M+H 401, M−H 399.

### (iii) 2,2,2-trifluoro-N-[(2S)-8-methoxy-5-({[3-(trifluoromethyl)phenyl]amino}sulfonyl)-1,2,3,4-tetrahydronaphthalen-2-yl]acetamide

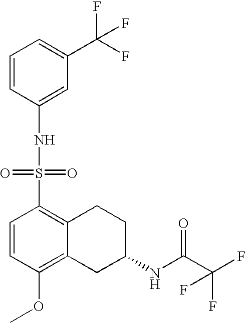

(6S)-4-methoxy-6-[(trifluoroacetyl)amino]-5,6,7,8-tetrahydronaphthalene-1-sulfonyl chloride (219 mg, 0.589 mmol) were dissolved in dichloromethane (2.5 ml). 3-Trifluoromethylaniline (104 mg, 0.648 mmol) and pyridine (0.072 ml, 0.884 mmol) were added and the reaction mixture was stirred for 16 hours. The organic phase was washed with hydrochloric acid (1 M), water, sat. aq. NaHCO₃ and dried (Na₂SO₄). The solvent was evaporated and the title compound (310 mg, 99%) was used in subsequent steps without further purification.

MS m/z M+H 497, M−H 495.

### Example 27

### (6S)-4-methoxy-N-phenyl-6-pyrrolidin-1-yl-5,6,7,8-tetrahydronaphthalene-1-sulfonamide

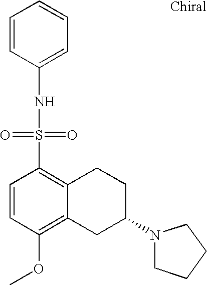

(6S)-6-Amino-4-methoxy-N-phenyl-5,6,7,8-tetrahydronaphthalene-1-sulfonamide (60 mg, 0.18 mmol), 1,4-dibromobutane (58 mg, 0.27 mmol), DIPEA (0.123 ml, 0.72 mmol) and potassium iodide (7.5 mg, 0.05 mmol) in toluene (2.5 ml) were heated to reflux for 20 hours. Dichloromethane (20 ml) were added and the organic phase was washed with citric acid (pH 4), water and dried over sodium sulfate. The solvent was removed under reduced pressure and the residue was purified by HPLC to give the title compound as a solid (10.2 mg, 15%).

¹H NMR (400 MHz, CDCl₃) δ ppm 7.90 (d, 1H) 7.16-7.23 (m, 2H) 7.07-7.13 (m, 2H) 7.00-7.06 (m, 1H) 6.70 (d, 1H) 3.84 (s, 3H) 3.62-3.73 (m, 1H) 3.09-3.36 (m, 6H) 2.97-3.09 (m, 1H) 2.77-2.92 (m, 1H) 2.35-2.45 (m, 1H) 1.92-2.15 (m, 5H). MS m/z M+H 387, M−H 385.

### Example 28

### (i) (6S)-6-[(2-fluoroethyl)amino]-4-methoxy-N-phenyl-5,6,7,8-tetrahydronaphthalene-1-sulfonamide

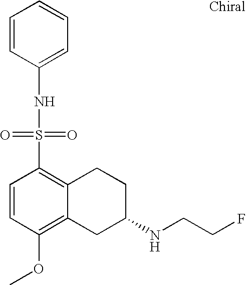

N-[(2S)-5-(anilinosulfonyl)-8-methoxy-1,2,3,4-tetrahydronaphthalen-2-yl]-2-fluoroacetamide (59 mg, 0.15 mmol) in THF (1 ml), was treated with borane tetrahydrofuran complex (0.6 ml, 1 M in THF, 0.6 mmol) under an atmosphere of argon. The reaction mixture was stirred at 50° C. for 16 hours. Another portion of borane tetrahydrofuran complex (0.6 ml, 1M in THF, 0.6 mmol) was added and the mixture was heated to reflux for 5 hours. The reaction mixture was cooled to room temperature and 5 M hydrochloric acid (0.72 ml) was carefully added. The reaction mixture was made basic by the addition of saturated aqueous NaHCO₃ solution, diluted with EtOAc and extracted with dichloromethane (3 times). The combined organic phase was dried (Na₂SO₄), filtered and the solvent was removed under reduced pressure. The residue was purified by HPLC to give the title compound (27 mg, 48%).

¹H NMR (400 MHz, CDCl₃) δ ppm 7.91 (d, 1H) 7.16-7.25 (m, 2H) 6.99-7.08 (m, 3H) 6.70 (d, 1H) 4.70 (t, 1H) 4.58 (t, 1H) 3.85 (s, 3H) 3.44-3.55 (m, 1H) 3.11-3.19 (m, 2H) 2.96-3.11 (m, 3H) 2.41 (dd, 1H) 2.11-2.19 (m, 1H) 1.60-1.73 (m, 1H). MS m/z M+H 379, M−H 377.

### (ii) N-[(2S)-5-(anilinosulfonyl)-8-methoxy-1,2,3,4-tetrahydronaphthalen-2-yl]-2-fluoroacetamide

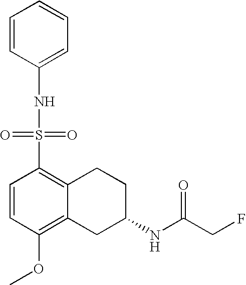

To a solution of fluoroacetic acid (15 mg, 0.19 mmol) in DMF (1 ml) were successively added hydroxybenzotriazole (25 mg, 0.19 mmol) diisopropylcarbodiimide (24 mg, 0.19 mmol), a solution of (6S)-6-Amino-4-methoxy-N-phenyl-5,6,7,8-tetrahydronaphthalene-1-sulfonamide (50 mg, 0.15 mmol) in DMF (2 ml) and DIPEA (0.099 ml, 0.6 mmol). The reaction mixture was stirred for 18 hours, the solvent was removed under reduced pressure and the residue was dissolved in dichloromethane. The organic phase was washed with hydrochloric acid (1M), water, sat. aqueous NaHCO₃ solution and dried (Na₂SO₄). The solvent was removed under reduced pressure and the residue purified by column chromatography on silica, using dichloromethane/methanol gradient mixtures (0-50% methanol) as eluent, to yield the title compound (65 mg, quant.).

¹H NMR (400 MHz, CDCl₃) δ ppm 7.94 (d, 1H) 7.19-7.26 (m, 2H) 7.05-7.11 (m, 1H) 6.97-7.03 (m, 2H) 6.72 (d, 1H) 4.88 (d, 1H) 4.76 (d, 1H) 4.21-4.32 (m, 1H) 3.86 (s, 3H) 3.40-3.52 (m, 1H) 3.04-3.21 (m, 2H) 2.47 (dd, 1H) 2.08-2.19 (m, 1H) 1.69-1.85 (m, 1H). MS m/z M+H 393, M−H 391.

### Example 29

### (i) (2S)-5-(2,3-dihydro-1H-indol-1-ylsulfonyl)-8-methoxy-N,N-dimethyl-1,2,3,4-tetrahydronaphthalen-2-amine

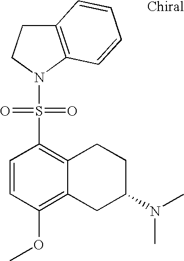

(2S)-5-(2,3-dihydro-1H-indol-1-ylsulfonyl)-8-methoxy-1,2,3,4-tetrahydronaphthalen-2-amine (107 mg, 0.299 mmol) in methanol (3 ml) was treated with paraformaldehyde (37% aq, 0.23 ml, 2.82 mmol), acetic acid (0.50 ml) and NaCNBH₃ (53 mg, 0.846 mmol). The reaction mixture was stirred for 10 hours. Dichloromethane was added and the organic layer was washed with sat. aq. NaHCO₃, dried (Na₂SO₄), filtered and concentrated. The residue was purified by HPLC to give the title compound as an acetate salt (41 mg, 36%). ¹H NMR (400 MHz, CDCl₃) δ ppm 7.88 (d, 1H) 7.24 (d, 1H) 7.15 (d, 1H) 7.09 (t, 1H) 6.95 (t, 1H) 6.75 (d, 1H) 3.96 (t, 2H) 3.87 (s, 3H) 3.58 (m, 1H) 3.17 (m, 1H) 3.00-3.10 and 3.06 (overlapping signals, m, 1H and t, 2H) 2.80-2.92 (m, 2H) 2.65 (s, 6H) 2.29 (m, 1H) 1.67 (m, 1H)

MS m/z M+H 387.

### (ii) (2S)-5-(2,3-dihydro-1H-indol-1-ylsulfonyl)-8-methoxy-1,2,3,4-tetrahydronaphthalen-2-amine

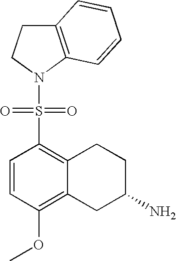

N-[(2S)-5-(2,3-dihydro-1H-indol-1-ylsulfonyl)-8-methoxy-1,2,3,4-tetrahydronaphthalen-2-yl]-2,2,2-trifluoroacetamide (136 mg, 0.299 mmol) in methanol (2 ml) and THF (2 ml) was treated with sodium hydroxide solution (1 ml, 2M). The reaction mixture was stirred for 16 hours. The pH was adjusted to 8 with hydrochloric acid (1 M) and sat. aq. NaHCO₃. The mixture was extracted with dichloromethane (5 times). The combined organic phase was dried (Na₂SO₄) and the solvent was removed under reduced pressure. The residue was used in the following reactions without further purification.

MS m/z M+H 359.

### (iii) N-[(2S)-5-(2,3-dihydro-1H-indol-1-ylsulfonyl)-8-methoxy-1,2,3,4-tetrahydronaphthalen-2-yl]-2,2,2-trifluoroacetamide

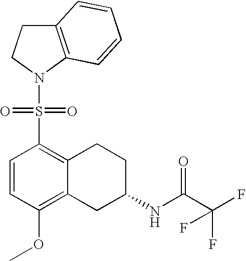

(6S)-4-methoxy-6-[(trifluoroacetyl)amino]-5,6,7,8-tetrahydronaphthalene-1-sulfonyl chloride (152 mg, 0.409 mmol) was dissolved in dichloromethane (2.5 ml). Indoline (54 mg, 0.449 mmol) and pyridine (0.83 ml, 1.02 mmol) were added and the reaction mixture was stirred for 16 hours. The organic phase was washed with hydrochloric acid (1 M), water, sat. aq. NaHCO₃ and dried (Na₂SO₄). The solvent was evaporated and the residue was crystallized from EtOAc/dichloromethane to give the title compound as a solid (139 mg, 75%).

MS m/z M+H 455, M−H 453.

### Example 30

### (i) (6S)—N-(5-chloro-2-methoxyphenyl)-4-methoxy-6-(methylamino)-5,6,7,8-tetrahydronaphthalene-1-sulfonamide

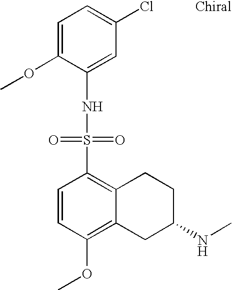

A solution of ethyl ((2S)-5-{[(5-chloro-2-methoxyphenyl)amino]sulfonyl}-8-methoxy-1,2,3,4-tetrahydronaphthalen-2-yl)carbamate (103 mg, 0.22 mmol) in THF (1 ml) was added dropwise to a suspension of LAH (26 mg, 0.66 mmol) in THF (1.5 ml). The resulting mixture was stirred for 3 hours at RT and then heated to reflux for 30 min. The reaction was quenched by careful addition of sat. aq. Na₂SO₄ (0.3 ml). The mixture was filtered and the solvent was removed under reduced pressure. The residue was purified by HPLC to give the title compound (54%).

¹H NMR (400 MHz, CDCl₃) δ ppm 7.93 (d, 1H) 7.31 (d, 1H) 6.93 (dd, 1H) 6.68-6.76 (m, 2H) 3.86 (s, 3H) 3.81 (s, 3H) 3.48-3.58 (m, 1H) 3.13-3.23 (m, 1H) 2.95-3.09 (m, 2H) 2.63 (br. s., 3H) 2.52 (dd, 1H) 2.22-2.32 (m, 1H) 1.66-1.80 (m, 1H). MS m/z M+H 411, 413, M−H 409, 411.

### (ii) ethyl ((2S)-5-{[(5-chloro-2-methoxyphenyl)amino]sulfonyl}-8-methoxy-1,2,3,4-tetrahydronaphthalen-2-yl)carbamate

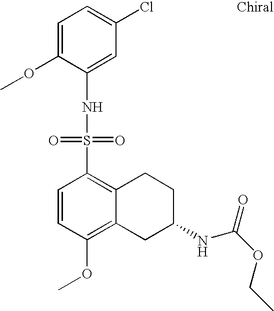

A suspension of (6S)-6-amino-N-(5-chloro-2-methoxyphenyl)-4-methoxy-5,6,7,8-tetrahydronaphthalene-1-sulfonamide (273 mg, 0.688 mmol) in dichloromethane (5 ml) was successively treated with triethylamine (0.24 ml) and ethyl chloroformate (0.069 ml, 0.722 mmol) and stirred for 1 hr. The mixture was diluted with dichloromethane, washed with hydrochloric acid (1 M), sat. aq. NaHCO₃ solution and dried (Na₂SO₄). The solvent was removed under reduced pressure and the residue was purified by chromatography on silica, using heptane/EtOAc gradient mixtures (0-100% EtOAc) as eluent to give the title compound as a solid (106 mg, 33%).

¹H NMR (400 MHz, CDCl₃) δ ppm 7.95 (d, 1H) 7.33-7.34 (m, 1H) 7.13 (s, 1H) 6.93 (dd, 1H) 6.72 (d, 1H) 6.71 (d, 1H) 4.07-4.19 (m, 2H) 3.88-3.98 (m, 1H) 3.36-3.50 (m, 1H) 3.04-3.18 (m, 2H) 2.42 (dd, 1H) 2.05-2.14 (m, 1H) 1.65-1.78 (m, 1H) 1.22-1.29 (m, 3H). MS m/z M+H 469, 471, M−H 467, 469.

### Example 31

### (i) (6S)—N-(4-chlorophenyl)-6-(dimethylamino)-4-methoxy-5,6,7,8-tetrahydronaphthalene-1-sulfonamide

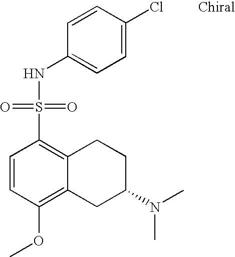

A solution of (6S)-6-amino-N-(4-chlorophenyl)-4-methoxy-5,6,7,8-tetrahydronaphthalene-1-sulfonamide (62 mg, 0.17 mmol) in methanol (5 ml) was successively treated with formaldehyde (37% aq., 136 mg, 1.67 mmol), acetic acid (151 mg) and NaCNBH₃ (32 mg, 0.51 mmol) and the reaction mixture was stirred for 10 hr. The solvent was removed under reduced pressure and the residue was dissolved in dichloromethane (10 ml). The organic phase was washed with sat. aq. NaHCO₃ solution (5 times), dried (Na₂SO₄) and the solvent was removed under reduced pressure.

The residue was purified by HPLC to give the title compound (31 mg, 47%).

¹H NMR (400 MHz, CDCl₃) δ ppm 7.90 (d, 1H) 7.10 (s, 4H) 6.71 (d, 1H) 3.86 (s, 3H) 3.77-3.86 (m, 1H) 3.36 (br. s., 1H) 3.05-3.13 (m, 1H) 2.97-3.05 (m, 1H) 2.70 (s, 6H) 2.44-2.59 (m, 2H) 1.67-1.81 (m, 1H). MS m/z M+H 395, 397, M−H 393, 395.

### (ii) (6S)-6-amino-N-(4-chlorophenyl)-4-methoxy-5,6,7,8-tetrahydronaphthalene-1-sulfonamide

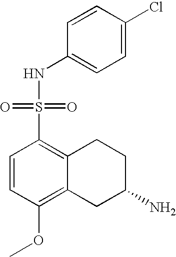

The method described in Example 29 (ii) was used to give the title compound (95%). MS m/z M+H 367, 369, M−H 365, 367.

### (iii) N-((2S)-5-{[(4-chlorophenyl)amino]sulfonyl}-8-methoxy-1,2,3,4-tetrahydronaphthalen-2-yl)-2,2,2-trifluoroacetamide

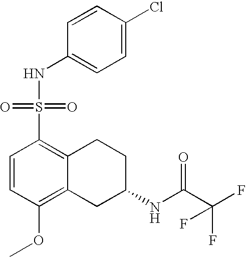

The method described in Example 29 (iii) was used to give the title compound (96%). MS m/z M+H 463, M−H 461, 463.

### Example 32

### (i) 2-{[(6S)-4-methoxy-6-pyrrolidin-1-yl-5,6,7,8-tetrahydronaphthalen-1-yl]sulfonyl}-1,2,3,4-tetrahydroisoquinoline-7-carbonitrile

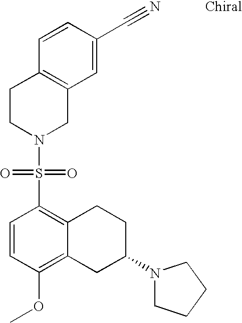

2-{[(6S)-6-amino-4-methoxy-5,6,7,8-tetrahydronaphthalen-1-yl]sulfonyl}-1,2,3,4-tetrahydroisoquinoline-7-carbonitrile (48 mg, 0.12 mmol), 1,4-dibromobutane (39 mg, 0.179 mmol), DIPEA (62 mg, 0.476 mmol) and potassium iodide (4 mg, 0.024 mmol) were suspended in 0.9 ml toluene and 0.1 ml N-methylpyrrolidine (NMP) and heated in a microwave oven for 40 min at 150° C. Ethyl acetate (10 ml) was added and the organic layer was successively washed with aqueous citric acid (pH 4), water, and sat. aqueous NaHCO₃ solution. The organic phase was dried (Na₂SO₄) and the solvent was removed under reduced pressure. The residue was purified by HPLC to give the product (20%).

¹H NMR (400 MHz, CDCl₃) δ ppm 7.95 (d, 1H) 7.46 (dd, 1H) 7.36 (br. s., 1H) 7.24 (d, 1H) 6.80 (d, 1H) 4.26-4.41 (m, 2H) 3.89 (s, 3H) 3.43-3.57 (m, 4H) 3.29-3.39 (m, 1H) 3.22-3.30 (m, 2H) 3.05-3.15 (m, 1H) 2.86-3.05 (m, 5H) 2.27-2.36 (m, 1H) 2.04-2.17 (m, 5H). MS m/z M+H 452.

### (ii) 2-{[(6S)-6-amino-4-methoxy-5,6,7,8-tetrahydronaphthalen-1-yl]sulfonyl}-1,2,3,4-tetrahydroisoquinoline-7-carbonitrile

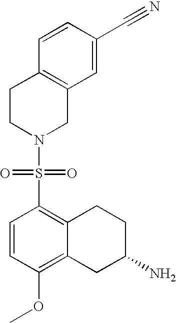

The method described in Example 29 (ii) was used to give the title compound (81%). MS m/z M+H 398.

### (iii) N-{(2S)-5-[(7-cyano-3,4-dihydroisoquinolin-2(1H)-yl)sulfonyl]-8-methoxy-1,2,3,4-tetrahydronaphthalen-2-yl}-2,2,2-trifluoroacetamide

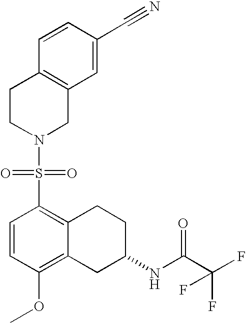

The method described in Example 29 (iii) was used to give the title compound (99%). MS m/z M+H 494, M−H 492.

### Example 33

### (6S)—N-(4-chlorophenyl)-4-methoxy-6-pyrrolidin-1-yl-5,6,7,8-tetrahydronaphthalene-1-sulfonamide

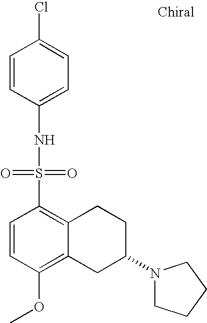

The method described in Example was used to give the title compound (58%).

¹H NMR (400 MHz, CDCl₃) δ ppm 7.87 (d, 1H) 7.01-7.13 (m, 4H) 6.69 (d, 1H) 3.78-3.84 (s, 3H) 3.62-3.76 (m, 1H) 3.22-3.36 (m, 4H) 3.09-3.21 (m, 2H) 2.96-3.10 (m, 1H) 2.69-2.82 (m, 1H) 2.36-2.45 (m, 1H) 2.04-2.11 (m, 4H) 1.88-2.00 (m, 1H). MS m/z M+H 421, 423, M−H 419, 421.

### Example 34

### (i) (6S)—N-(3,4-dichlorophenyl)-4-methoxy-6-pyrrolidin-1-yl-5,6,7,8-tetrahydronaphthalene-1-sulfonamide

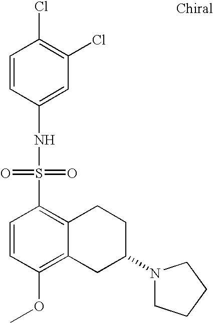

(6S)-6-amino-N-(3,4-dichlorophenyl)-4-methoxy-5,6,7,8-tetrahydronaphthalene-1-sulfonamide (78 mg, 0.194 mmol), 1,4-dibromobutane and DIPEA (0.096 ml, 0.582 mmol) in acetonitril (0.5 ml) were heated in a microwave oven for 15 min at 130° C. The reaction mixture was purified by HPLC to give the title compound (8 mg, 9%).

¹H NMR (400 MHz, CDCl₃) δ ppm 7.92 (d, 1H) 7.37 (d, 1H) 7.20 (d, 1H) 7.10 (dd, 1H) 6.72 (d, 1H) 3.82-3.87 (m, 3H) 3.70-3.80 (m, 1H) 2.96-3.44 (m, 7H) 2.69-2.83 (m, 1H) 2.41-2.52 (m, 1H) 2.05-2.16 (m, 4H) 1.87-2.00 (m, 1H). MS m/z M+H 455, 457, 459, M−H 453, 455, 457.

### (ii) (6S)-6-amino-N-(3,4-dichlorophenyl)-4-methoxy-5,6,7,8-tetrahydronaphthalene-1-sulfonamide

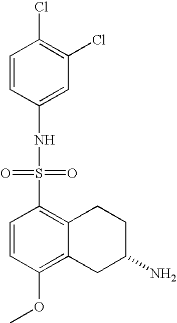

(6S)-4-methoxy-6-[(trifluoroacetyl)amino]-5,6,7,8-tetrahydronaphthalene-1-sulfonyl chloride (103 mg, 0.277 mmol), 3,4-dichloroaniline (46 mg, 0.284 mmol) and pyridine (0.056 ml) were dissolved in THF (0.5 ml) and dichloromethane (1 ml). The reaction mixture was stirred for 2 hours. Sodium hydroxide (2 M, 0.663 ml) were added and the reaction mixture was stirred for 10 hours. Sat. aqueous NH₄Cl solution was added until pH ˜9 was reached. A precipitate formed which was dissolved in THF. The aqueous layer was extracted with THF and dichloromethane. The combined organic phase was dried (Na₂SO₄), and the solvent was removed under reduced pressure to give the title compound (121 mg, 99%), which was used without further purification in subsequent reaction steps. MS m/z M+H 401, 403, M−H 399, 401.

### Example 35

### (i) (6S)—N-(3,4-difluorophenyl)-4-methoxy-6-pyrrolidin-1-yl-5,6,7,8-tetrahydronaphthalene-1-sulfonamide

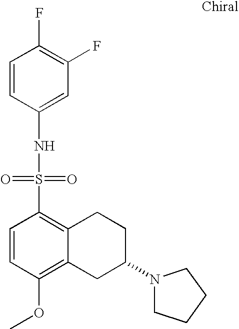

(6S)-6-amino-N-(3,4-difluorophenyl)-4-methoxy-5,6,7,8-tetrahydronaphthalene-1-sulfonamide (63 mg, 0.171 mmol), 1,4-dibromobutane and DIPEA (0.021 ml, 0.180 mmol) in acetonitril (0.5 ml) were heated in a microwave oven for 10 min at 130° C. The reaction mixture was purified by HPLC to give the title compound (38 mg, 53%).

¹H NMR (400 MHz, CDCl₃) δ ppm 7.87 (d, 1H) 7.15 (m, 1H) 6.87-7.00 (m, 2H) 6.70 (d, 1H) 3.80-3.90 and 3.84 (overlapping signals, m, 1H and s, 3H) 3.37-3.56 (m, 4H) 3.18 (m, 1H) 3.02-3.14 (m, 1H) 2.83 (dd, 1H) 2.51-2.60 (m, 1H) 2.17 ((m, 4H) 1.98-2.07 (m, 1H)

MS m/z M+H 423, M−H 421.

### (ii) (6S)-6-amino-N-(3,4-difluorophenyl)-4-methoxy-5,6,7,8-tetrahydronaphthalene-1-sulfonamide

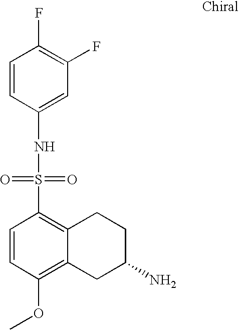

The method described in Example 34 (ii) was used to give the title compound (50%).

MS m/z M+H 369, M−H 367.

### Example 36

### (i) (6S)—N-(5-chloropyridin-2-yl)-6-(dimethylamino)-4-methoxy-5,6,7,8-tetrahydronaphthalene-1-sulfonamide

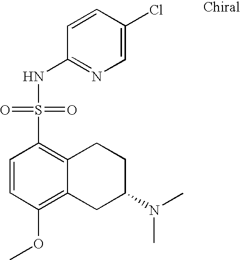

The method described in Example 19 (i) was used to give the title compound (13%).

¹H NMR (400 MHz, DMSO-d₆) δ ppm 8.08-8.16 (m, 1H) 7.82-7.87 (d, 1H) 7.61-7.70 (m, 1H) 6.88-6.99 (m, 2H) 3.81-3.85 (br s, 3H) 3.43-3.53 (m, 1H) 2.75-2.88 (m, 2H) 2.45-2.60 (m, 2H) 2.23-2.29 (br s, 6H) 1.93-2.04 (m, 1H) 1.37-1.55 (m, 1H). MS m/z M+H 396.2, 398.1.

### (ii) N-((2S)-5-{[(5-chloropyridin-2-yl)amino]sulfonyl}-8-methoxy-1,2,3,4-tetrahydronaphthalen-2-yl)-2,2,2-trifluoroacetamide

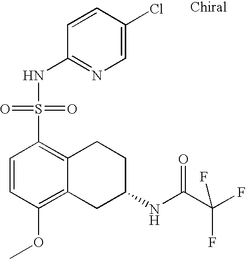

The method described in Example 19 (ii) was used to give the title compound (17%).

MS m/z M+H 463.

### Example 37

### (i) (6S)-6-(dimethylamino)-4-methoxy-N-pyridin-3-yl-5,6,7,8-tetrahydronaphthalene-1-sulfonamide

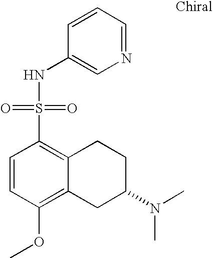

The method described in Example 19 (i) was used to give the title compound (6%).

¹H NMR (400 MHz, CD₃OD) δ ppm 8.20-8.25 (m, 1H) 8.13-8.18 (m, 1H) 7.89-7.94 (d, 1H) 7.52-7.56 (m, 1H) 7.23-7.29 (m, 1H) 6.90-6.95 (d, 1H) 3.87-3.92 (br s, 3H) 3.63-3.73 (m, 1H) 3.12-3.25 (m, 2H) 2.91-3.03 (m, 1H) 2.69-2.74 (br s, 6H) 2.58-2.68 (m, 1H) 2.27-2.36 (m, 1H) 1.65-1.78 (m, 1H).

MS m/z M+H 362.2.

### (ii) 2,2,2-trifluoro-N-{(2S)-8-methoxy-5-[(pyridin-3-ylamino)sulfonyl]-1,2,3,4-tetrahydronaphthalen-2-yl}acetamide

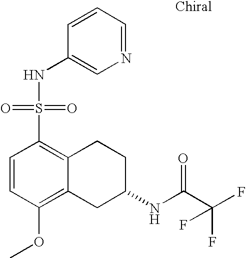

The method described in Example 19 (ii) was used to give the title compound (78%).

MS m/z M+H 430.2.

### Example 38

### (i) (6S)—N-1,3-benzodioxol-5-yl-4-methoxy-6-pyrrolidin-1-yl-5,6,7,8-tetrahydronaphthalene-1-sulfonamide

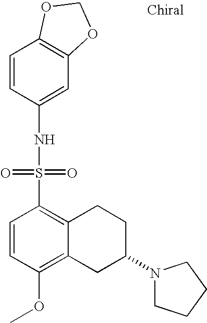

The method described in Example 35 (i) was used give the title compound (27%).

¹H NMR (400 MHz, CDCl₃) δ ppm 7.79 (d, 1H) 6.65-6.68 (m, 2H) 6.57 (d, 1H) 6.46 (dd, 1H) 5.88 (s, 2H) 3.84 (s, 3H) 3.57-3.67 (m, 1H) 3.21-3.29 (m, 4H) 2.95-3.21 (m, 3H) 2.77 (dd, 1H) 2.31-2.39 (m, 1H) 2.02-2.07 (m, 4H) 1.81-1.98 (m, 1H). MS m/z M+H 430.9, M−H 429.0.

### (ii) (6S)-6-amino-N-1,3-benzodioxol-5-yl-4-methoxy-5,6,7,8-tetrahydronaphthalene-1-sulfonamide

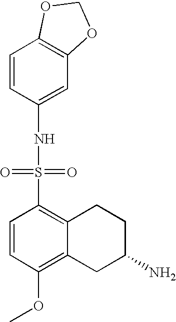

The method described in Example 34 (ii was used to give the title compound (90%)

MS m/z M+H 377, M−H 375.

### Example 39

### (i) (6S)—N-(5-chloro-2-methoxyphenyl)-6-[(2-fluoroethyl)amino]-4-methoxy-5,6,7,8-tetrahydronaphthalene-1-sulfonamide

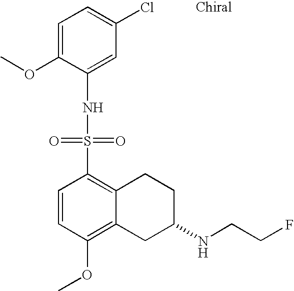

The method described in Example 28 (i) was used to give the title compound (96%).

¹H NMR (400 MHz, CDCl₃) δ ppm 7.91 (d, 1H) 7.31-7.36 (m, 1H) 6.93 (dd, 1H) 6.67-6.74 (m, 2H) 4.67 (t, 1H) 4.55 (t, 1H) 3.85 (s, 3H) 3.81 (s, 3H) 3.40-3.51 (m, 1H) 2.89-3.15 (m, 5H) 2.35 (dd, 1H) 2.08-2.17 (m, 1H) 1.55-1.68 (m, 1H). MS m/z M+H 442.9, 444.9, M−H 440.9, 442.9.

### (ii) N-((2S)-5-{[(5-chloro-2-methoxyphenyl)amino]sulfonyl}-8-methoxy-1,2,3,4-tetrahydronaphthalen-2-yl)-2-fluoroacetamide

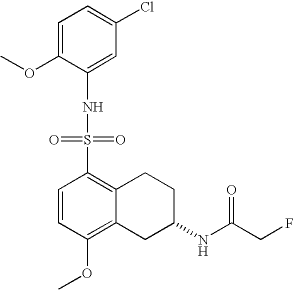

The method described in Example 28 (ii) was used to give the title compound (90%).

¹H NMR (400 MHz, CDCl₃) δ ppm 7.97 (d, 1H) 7.32 (d, 1H) 7.14 (s, 1H) 6.93 (dd, 1H) 6.75 (d, 1H) 6.71 (d, 1H) 6.23-6.34 (m, 1H) 4.80 (d, 2H) 4.20-4.31 (m, 1H) 3.86 (s, 3H) 3.83 (s, 3H) 3.43-3.53 (m, 1H) 3.05-3.20 (m, 2H) 2.45 (dd, 1H) 2.10-2.20 (m, 1H) 1.69-1.83 (m, 1H). MS m/z M+H 456.9, 457.9, M−H 454.9, 456.9.

### Example 40

### (6S)—N-(5-chloro-2-methoxyphenyl)-6-[(2-fluoroethyl)(ethyl)amino]-4-methoxy-5,6,7,8-tetrahydronaphthalene-1-sulfonamide

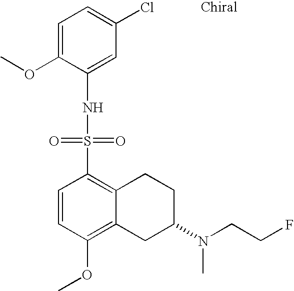

(6S)—N-(5-chloro-2-methoxyphenyl)-6-[(2-fluoroethyl)amino]-4-methoxy-5,6,7,8-tetrahydronaphthalene-1-sulfonamide (167 mg, 0.377 mmol) were dissolved in methanol (5 ml). Formaldehyde (37% aq., 306 mg, 3.77 mmol), acetic acid (0.108 ml) and NaCNBH₃ (71 mg, 1.13 mmol) were added and the reaction mixture was stirred for 30 min. The solvent was removed under reduced pressure. The residue was dissolved in dichloromethane and washed with sat. aqueous NaHCO₃ solution. The organic phase was dried (Na₂SO₄) and the solvent was removed under reduced pressure. The residue was purified by column chromatography on silica, using dichloromethane/methanol gradient mixtures (0-20% methanol) to elute the title compound (96 mg, 56%).

¹H NMR (400 MHz, CDCl₃) δ ppm 7.92 (d, 1H) 7.32 (d, 1H) 7.14 (s, 1H) 6.92 (dd, 1H) 6.68-6.74 (m, 2H) 4.59-4.67 (m, 1H) 4.48-4.56 (m, 1H) 3.96-4.08 (m, 1H) 3.86 (s, 3H) 3.82-3.86 (m, 1H) 3.81 (s, 3H) 3.52-3.62 (m, 1H) 2.78-3.03 (m, 4H) 2.45 (s, 3H) 2.08-2.19 (m, 1H) 1.53-1.66 (m, 1H). MS m/z M+H 456.9, 458.9, M−H 454.8, 456.8.

### Example 41

### (i) (6S)-4-methoxy-6-(methylamino)-N-[4-(trifluoromethyl)phenyl]-5,6,7,8-tetrahydronaphthalene-1-sulfonamide

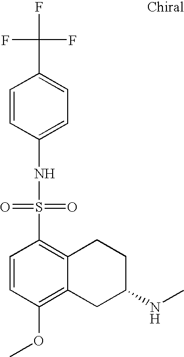

The method described in Example 16 (i) was used to give the title compound (55%).

¹H NMR (400 MHz, CDCl₃) δ ppm 7.95 (d, 1H) 7.46 (d, 2H) 7.11 (d, 2H) 6.73 (d, 1H) is 3.87 (s, 3H) 3.40-3.51 (m, 1H) 2.96-3.09 (m, 2H) 2.78-2.88 (m, 1H) 2.54 (s, 3H) 2.30-2.40 (m, 1H) 2.07-2.15 (m, 1H) 1.53-1.67 (m, 1H). MS m/z M+H 414.8, M−H 412.8.

### (ii) ethyl [(2S)-8-methoxy-5-({[4-(trifluoromethyl)phenyl]amino}sulfonyl)-1,2,3,4-tetrahydronaphthalen-2-yl]carbamate

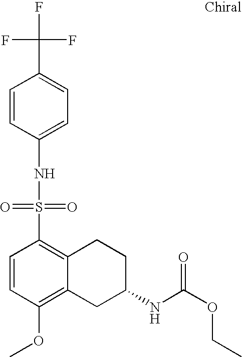

(6S)-6-amino-4-methoxy-N-[4-(trifluoromethyl)phenyl]-5,6,7,8-tetrahydronaphthalene-1-sulfonamide (174 mg, 0.436 mmol), ethyl chloroformate (0.42 ml, 0.440 mmol) and pyridine (0.052 ml, 0.654 mmol) were stirred in dichloromethane (1.5 ml) for 24 hours. Additional dichloromethane (10 ml) was added and the reaction mixture was successively washed with hydrochloric acid (1 M), water and sat. aqueous NaHCO₃ solution. The organic phase was dried (Na₂SO₄) and the solvent was removed under reduced pressure to give the title compound (179 mg, 87%), which was used in subsequent reaction step without further purification.

¹H NMR (400 MHz, CDCl₃) δ ppm 7.98 (d, 1H) 7.46 (d, 2H) 7.12 (d, 2H) 6.75 (d, 1H) 4.63-4.74 (m, 1H) 4.09-4.20 (m, 2H) 3.89-3.98 (m, 1H) 3.86 (s, 3H) 3.39-3.51 (m, 1H) 3.03-3.17 (m, 2H) 2.41 (dd, 1H) 2.06-2.16 (m, 1H) 1.67-1.79 (m, 1H) 1.22-1.32 (m, 3H). MS m/z M+NH₄ 489.9, M+H 472.8, M−H 470.8.

### (iii) (6S)-6-amino-4-methoxy-N-[4-(trifluoromethyl)phenyl]-5,6,7,8-tetrahydronaphthalene-1-sulfonamide

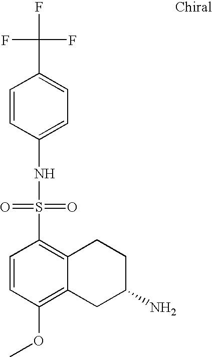

The method described in Example 34 (ii) was used to give the title compound (95%).

¹H NMR (400 MHz, CDCl₃) δ ppm 7.96 (d, 1H) 7.43 (d, 2H) 7.10 (d, 2H) 6.72 (d, 1H) 3.84 (s, 3H) 3.41-3.53 (m, 1H) 3.09-3.21 (m, 1H) 2.97-3.09 (m, 2H) 2.31 (dd, 1H) 1.99-2.12 (m, 1H) 1.52-1.69 (m, 1H). MS m/z M+H 400.9, M−H 398.9.

### Example 42

### (i) (6S)—N-(4-chlorophenyl)-4-methoxy-N-methyl-6-(methylamino)-5,6,7,8-tetrahydronaphthalene-1-sulfonamide

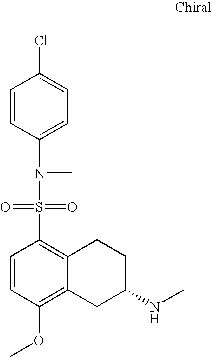

N-((2S)-5-{[(4-chlorophenyl)(methyl)amino]sulfonyl}-8-methoxy-1,2,3,4-tetrahydronaphthalen-2-yl)-2,2,2-trifluoro-N-methylacetamide (93 mg, 0.19 mmol), ammonia in methanol (7 M, 0.270 ml), water (0.06 ml) and methanol (1 ml) were heated in a microwave oven at 140° C. for 1.5 hr. The solvent was removed under reduced pressure and the residue was purified by column chromatography on silica using dichloromethane/methanol gradient mixtures (0-20% methanol) as eluent to yield the title compound (69 mg, 92%).

¹H NMR (400 MHz, CDCl₃) δ ppm 7.76 (d, 1H) 7.23-7.29 (m, 2H) 7.08-7.13 (m, 2H) 6.74 (d, 1H) 3.86 (s, 3H) 3.30 (dd, 1H) 3.16 (s, 3H) 3.12-3.25 (m, 2H) 2.75 (s, 3H) 2.61-2.81 (m, 2H) 2.15-2.25 (m, 1H) 1.62-1.77 (m, 1H)

MS m/z M+H 394.8, 396.8.

### (ii) N-((2S)-5-{[(4-chlorophenyl)(methyl)amino]sulfonyl}-8-methoxy-1,2,3,4-tetrahydronaphthalen-2-yl)-2,2,2-trifluoro-N-methylacetamide

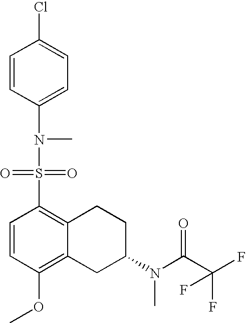

(6S)-4-methoxy-6-[methyl(trifluoroacetyl)amino]-5,6,7,8-tetrahydronaphthalene-1-sulfonyl chloride (304 mg, 0.787 mmol), N-methyl-p-chloroaniline (223 mg, 1.575 mmol) and pyridine (0.064 ml, 0.787 mmol) in dichloromethane (5 ml) were stirred for 10 hours. The solvent was removed under reduced pressure and the residue was purified using a SCX-2 column eluting the product with dichloromethane, dichloromethane/methanol (2%), and dichloromethane/methanol (4%) to give the title compound (393 mg, 99%).

MS m/z M+H 490.7, 492.7.

### (iii) (6S)-4-methoxy-6-[methyl(trifluoroacetyl)amino]-5,6,7,8-tetrahydronaphthalene-1-sulfonyl chloride

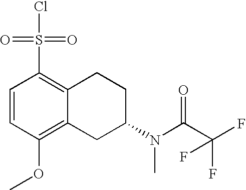

A solution of chlorosulfonic acid (4.93 ml, 73.84 mmol) in dichloromethane (20 ml) was added to a solution of (2,2,2-trifluoro-N-[(2S)-8-methoxy-1,2,3,4-tetrahydronaphthalen-2-yl]-N-methylacetamide (5.3 g, 18.5 mmol) in dichloromethane (100 ml). The mixture was stirred for 10 hours at RT. The reaction mixture was poured on ice and the product was extracted with dichloromethane. The organic phase was washed with sat. aqueous NaHCO₃ solution and dried (Na₂SO₄). The solvent was removed to give the title compound, which was used without further purification.

¹H NMR (400 MHz, CDCl₃) mixture of rotamers δ ppm 8.04 (m, 1H) 6.85 (m, 1H) 4.68-4.78 and 4.17-4.26 (m, 1H) 3.96 and 3.94 (s and s, 3H) 3.72-3.83 (m, 1H) 3.12-3.27 (m, 1H) 3.09-3.11 and 3.03 (m and m, 3H) 2.99-3.09 (m, 1H) 2.80 and 2.63 (m and m, 1H) 2.04-2.18 (m, 1H) 1.91-2.04 (m, 1H)

MS m/z M+H 384.9

### (iv) 2,2,2-trifluoro-N-[(2S)-8-methoxy-1,2,3,4-tetrahydronaphthalen-2-yl]-N-methylacetamide

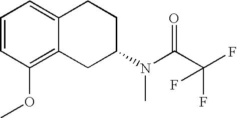

Trifluoroacetic anhydride (3.23 ml, 22.9 mmol) was slowly added to a stirred solution of (2S)-8-methoxy-N-methyl-1,2,3,4-tetrahydronaphthalen-2-amine (4.17 g, 21.78 mmol) and pyridine (2.64 ml, 32.7 mmol) in dichloromethane (50 ml). The mixture was stirred for 30 min after addition, then diluted with dichloromethane (100 ml) and successively washed with hydrochloric acid (1 M), water and sat. aqueous NaHCO₃. The organic phase was dried (Na₂SO₄) and the solvent was removed under reduced pressure. The residue was purified by chromatography on silica using a gradient of heptane/ethyl acetate reaching from 0-100% of ethyl acetate. The title compound was isolated as oil (5.3 g, 85%).

¹H NMR (400 MHz, CDCl₃) mixture of rotamers δ ppm 7.15 (m, 1H) 6.66-6.79 (m, 2H) 4.77 and 4.20 (m, 1H), 3.84 and 3.83 (s, 3H) 2.89-3.10 (m, 6H) 2.76 and 2.60 (m, 1H) 1.84-2.03 (m, 2H)

### (v) (2S)-8-methoxy-N-methyl-1,2,3,4-tetrahydronaphthalen-2-amine

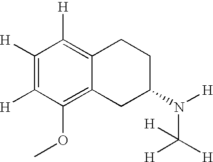

The method described in Example 16 (i) was used to give the title compound (99%)

¹H NMR (400 MHz, CDCl₃) δ ppm 7.10 (t, 1H) 6.73 (d, 1H) 6.67 (d, 1H) 3.81-3.84 (m, 3H) 3.08 (dd, 1H) 2.76-2.94 (m, 3H) 2.55 (s, 3H) 2.33 (dd, 1H) 2.01-2.10 (m, 1H) 1.56 (m, 1H)

MS m/z M+H 192.2.

### Example 43

### (2S)-5-(1H-indol-1-ylsulfonyl)-8-methoxy-N-methyl-1,2,3,4-tetrahydronaphthalen-2-amine

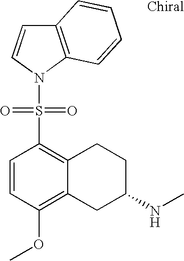

DMF (1 ml) was added to indole (17 mg, 0.142 mmol) and NaH (10 mg, 0.416 mmol) and the mixture was stirred for 15 min. A solution of (6S)-4-methoxy-6-[methyl(trifluoroacetyl)amino]-5,6,7,8-tetrahydronaphthalene-1-sulfonyl chloride (50 mg, 0.13 mmol) in DMF (1 ml) was added and the resulting reaction mixture was stirred for 10 hours. Excess of NaH was destroyed by addition of 0.1 ml of water. The solvent was removed under reduced pressure and the residue was purified by HPLC to give the title compound (22 mg, 46%).

¹H NMR (400 MHz, CDCl₃) δ ppm 7.85 (d, 1H) 7.62-7.70 (m, 2H) 7.56-7.61 (m, 1H) 7.19-7.26 (m, 2H) 6.74 (d, 1H) 6.67 (d, 1H) 3.84 (s, 3H) 3.28-3.38 (m, 1H) 3.02 (dd, 1H) 2.63-2.78 (m, 2H) 2.51 (s, 3H) 2.32 (dd, 1H) 2.01-2.10 (m, 1H) 1.43-1.58 (m, 1H).

MS m/z M+H 370.9, M−H 369.

### Example 44

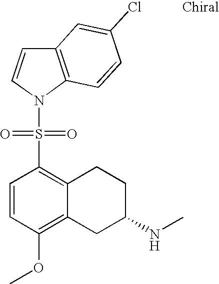

### (2S)-5-[(5-chloro-1H-indol-1-yl)sulfonyl]-8-methoxy-N-methyl-1,2,3,4-tetrahydronaphthalen-2-amine

DMF (1 ml) was added to 5-chloroindole (44 mg, 0.28 mmol) and NaH (10 mg, 0.416 mmol) and the mixture was stirred for 15 min. A solution of (6S)-4-methoxy-6-[methyl(trifluoroacetyl)amino]-5,6,7,8-tetrahydronaphthalene-1-sulfonyl chloride (100 mg, 0.26 mmol) in DMF (1 ml) was added and the resulting reaction mixture was stirred for 10 hours.

Ammonia in methanol (7 M, 1 ml) was added and the reaction mixture was stirred for another 10 hours. The solvent was removed under reduced pressure and the residue was purified by HPLC to give the title compound (50 mg, 47%).

¹H NMR (400 MHz, CDCl₃) □ ppm 7.82 (d, 1H) 7.65 (d, 1H) 7.60 (d, 1H) 7.55 (d, 1H) 7.18 (dd, 1H) 6.74 (d, 1H) 6.60 (d, 1H) 3.86 (s, 3H) 3.22-3.31 (m, 1H) 2.97 (dd, 1H) 2.61-2.72 (m, 2H) 2.46 (s, 3H) 2.25 (dd, 1H) 1.98 (dd, 1H) 1.37-1.53 (m, 1H). MS m/z M+H 404.7, 406.7, M−H 402.8, 404.8.

### Example 45

### (2S)-8-methoxy-N-methyl-5-{[6-(trifluoromethyl)-1H-indol-1-yl]sulfonyl}-1,2,3,4-tetrahydronaphthalen-2-anine

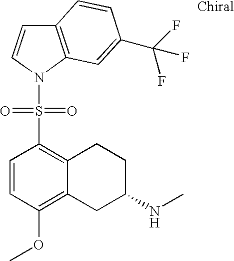

The method described in Example 44 was used to give the title compound (46%)

¹H NMR (400 MHz, CDCl₃) δ ppm 7.99 (s, 1H) 7.91 (d, 1H) 7.77 (d, 1H) 7.68 (d, 1H) 7.47 (dd, 1H) 6.78 (d, 1H) 6.72 (dd, 1H) 3.87 (s, 3H) 3.24-3.33 (m, 1H) 3.00 (dd, 1H) 2.65-2.77 (m, 2H) 2.48 (s, 3H) 2.29 (dd, 1H) 1.97-2.06 (m, 1H) 1.41-1.54 (m, 1H) MS m/z M+H 438.7, M−H 436.7.

### Example 46

### 1-{[(6S)-4-methoxy-6-(methylamino)-5,6,7,8-tetrahydronaphthalen-1-yl]sulfonyl}-1H-indole-6-carbonitrile

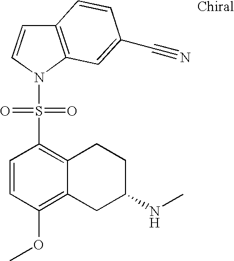

The method described in Example 44 was used to give the title compound (36%).

¹H NMR (400 MHz, CDCl₃) δ ppm 7.95-8.00 (m, 2H) 7.84 (d, 1H) 7.67 (d, 1H) 7.47 (dd, 1H) 6.83 (d, 1H) 6.73 (d, 1H) 3.91 (s, 3H) 3.18-3.27 (m, 1H) 2.99 (dd, 1H) 2.60-2.72 (m, 2H) 2.47 (s, 3H) 2.27 (dd, 1H) 1.94-2.02 (m, 1H) 1.39-1.51 (m, 1H). MS m/z M+H 395.8, M−H 393.9.

### Example 47

### (2S)-5-[(7-fluoro-1H-indol-1-yl)sulfonyl]-8-methoxy-N-methyl-1,2,3,4-tetrahydronaphthalen-2-amine

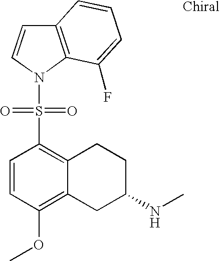

The method described in Example 44 was used to give the title compound (40%).

¹H NMR (400 MHz, CDCl₃) δ ppm 8.15 (dd, 1H) 7.83 (d, 1H) 7.34 (d, 1H) 7.08-7.15 (m, 1H) 6.90 (m, 1H) 6.80 (d, 1H) 6.67 (dd, 1H) 3.89 (s, 3H) 3.17-3.27 (m, 1H) 3.00 (dd, 1H) 2.56-2.70 (m, 2H) 2.46 (s, 3H) 2.25 (dd, 1H) 1.94-2.03 (m, 1H) 1.37-1.50 (m, 1H). MS m/z M+H 388.8, M−H 386.9.

### Example 48

### (2S)-5-[(4-fluoro-1H-indol-1-yl)sulfonyl]-8-methoxy-N-methyl-1,2,3,4-tetrahydronaphthalen-2-amine

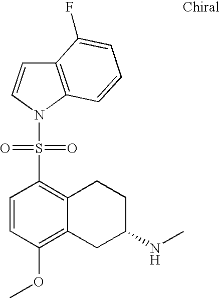

The method described in Example 44 was used to give the title compound (47%).

¹H NMR (400 MHz, CDCl₃) δ ppm 7.87 (d, 1H) 7.62 (d, 1H) 7.46 (d, 1H) 7.12-7.20 (m, 1H) 6.87-6.93 (m, 1H) 6.73-6.78 (m, 2H) 3.87 (s, 3H) 3.23-3.33 (m, 1H) 2.98 (dd, 1H) 2.62-2.73 (m, 2H) 2.47 (s, 3H) 2.26 (dd, 1H) 1.95-2.04 (m, 1H) 1.39-1.51 (m, 1H). MS m/z M+H 388.8, M−H 386.9

### Example 49

### (2S)-8-methoxy-5-[(4-methoxy-1H-indol-1-yl)sulfonyl]-N-methyl-1,2,3,4-tetrahydronaphthalen-2-amine

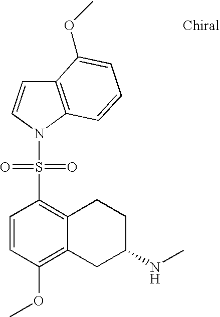

The method described in Example 44 was used to give the title compound (27%).

¹H NMR (400 MHz, CDCl₃) δ ppm 7.82 (d, 1H) 7.54 (d, 1H) 7.27 (d, 1H) 7.15 (t, 1H) 6.78 (d, 1H) 6.73 (d, 1H) 6.64 (d, 1H) 3.93 (s, 3H) 3.84 (s, 3H) 3.28-3.38 (m, 1H) 3.02 (dd, 1H) 2.61-2.76 (m, 2H) 2.50 (s, 3H) 2.31 (dd, 1H) 2.00-2.09 (m, 1H) 1.43-1.55 (m, 1H)

MS m/z M+H 400.7, M−H 398.7.

### Example 50

### (2S)-5-(5H-[1,3]dioxolo[4,5-f]indol-5-ylsulfonyl)-8-methoxy-N-methyl-1,2,3,4-tetrahydronaphthalen-2-amine

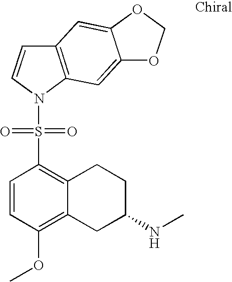

The method described in Example 44 was used to give the title compound (11%).

¹H NMR (400 MHz, CDCl₃) δ ppm 7.75 (d, 1H) 7.49 (d, 1H) 7.15 (s, 1H) 6.93 (s, 1H) 6.74 (d, 1H) 6.53 (d, 1H) 5.95 (s, 2H) 3.85 (s, 3H) 3.29-3.39 (m, 1H) 3.07 (dd, 1H) 2.75-2.85 (m, 1H) 2.63-2.75 (m, 1H) 2.54 (s, 3H) 2.39 (dd, 1H) 2.07-2.16 (m, 1H) 1.50-1.64 (m, 1H)

MS m/z M+H 414.6, M−H 412.8.

### Example 51

### (2S)-5-[(7-chloro-1H-indol-1-yl)sulfonyl]-8-methoxy-N-methyl-1,2,3,4-tetrahydronaphthalen-2-amine

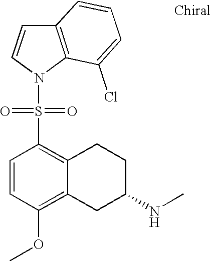

The method described in Example 44 was used to give the title compound (46%).

¹H NMR (400 MHz, CDCl₃) δ ppm 7.89 (d, 1H) 7.79 (d, 1H) 7.50 (dd, 1H) 7.20-7.24 (m, 1H) 7.14 (t, 1H) 6.72 (d, 1H) 6.70 (d, 1H) 3.87 (s, 3H) 3.22-3.31 (m, 1H) 3.08 (dd, 1H) 2.63-2.79 (m, 2H) 2.51 (s, 3H) 2.33 (dd, 1H) 2.02-2.11 (m, 1H) 1.47-1.59 (m, 1H). MS m/z M+H 404.7, 406.7, M−H 402.8, 404.8.

### Example 52

### (i) (2S)-8-methoxy-N-methyl-5-(1H-pyrrolo[2,3-b]pyridin-1-ylsulfonyl)-1,2,3,4-tetrahydronaphthalen-2-amine

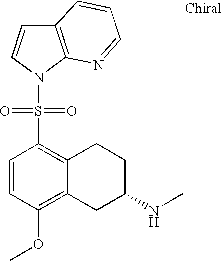

2,2,2-trifluoro-N-[(2S)-8-methoxy-5-(1H-pyrrolo[2,3-b]pyridin-1-ylsulfonyl)-1,2,3,4-tetrahydronaphthalen-2-yl]-N-methylacetamide (130 mg, 0.278 mmol) was mixed with ammonia in methanol (7 M, 2 ml) and the reaction mixture was stirred in a sealed vial for 10 hours. The solvent was removed under reduced pressure and the residue was purified by chromatography on silica using a gradient of CHCl₃/MeOH/NH₃ reaching from 0-10% of methanol containing ammonia (3%) to yield the title compound (41 mg, 40%). ¹H NMR (400 MHz, CDCl₃) δ ppm 8.26-8.34 (m, 2H) 7.86 (dd, 1H) 7.82 (d, 1H) 7.14 (dd, 1H) 6.81 (d, 1H) 6.60 (d, 1H) 3.87 (s, 3H) 3.23-3.34 (m, 1H) 2.98 (dd, 1H) 2.71-2.82 (m, 1H) 2.63-2.70 (m, 1H) 2.46 (s, 3H) 2.23 (dd, 1H) 1.93-2.02 (m, 1H) 1.35-1.49 (m, 1H)

MS m/z M+H 372, M−H 370.

### (ii) 2,2,2-trifluoro-N-[(2S)-8-methoxy-5-(1H-pyrrolo[2,3-b]pyridin-1-ylsulfonyl)-1,2,3,4-tetrahydronaphthalen-2-yl]-N-methylacetamide

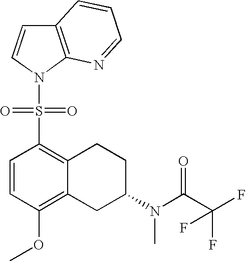

A solution of 7-azaindole in DMF (1 ml) was added to a suspension of NaH (14 mg, 0.591 mmol) in DMF (0.5 ml). The mixture was stirred for 15 min and then a solution of (6S)-4-methoxy-6-[methyl(trifluoroacetyl)amino]-5,6,7,8-tetrahydronaphthalene-1-sulfonyl chloride (152 mg, 0.394 mmol) in DMF (1 ml) was added and the reaction mixture was stirred for 10 hours. Water (0.05 ml) was added and the solvent was removed under reduced pressure. The residue was dissolved in dichloromethane and the organic layer was successively washed with sat. aqueous NaHCO₃, water and dried (Na₂SO₄). The solvent was removed under reduced pressure and the residue was purified by column chromatography on silica, using methanol in dichloromethane gradient mixtures (0-20% methanol) as eluent to yield the title compound (130 mg, 70%).

MS m/z M+H 467.7, M−H 465.8.

### Example 53

### (i) (6S)-6-(dimethylamino)-4-methoxy-N-quinolin-3-yl-5,6,7,8-tetrahydronaphthalene-1-sulfonamide

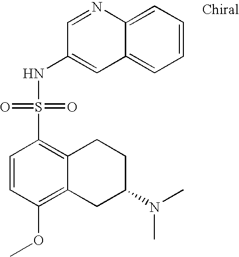

The method described in Example 31 (i) was used to give the title compound (31%).

¹H NMR (400 MHz, CDCl₃) δ ppm 9.22 (s, 1H) 8.07 (d, 1H) 7.91 (d, 1H) 7.69 (d, 1H) 7.58-7.64 (m, 1H) 7.55 (s, 1H) 7.41-7.47 (m, 1H) 6.70 (d, 1H) 3.82 (s, 3H) 3.55-3.64 (m, 1H) 2.93-3.02 (m, 1H) 2.87-2.94 (m, 1H) 2.32-2.48 (m, 2H) 2.30 (s, 6H) 1.89-1.97 (m, 1H) 1.34-1.48 (m, 1H). MS m/z M+H 412.3. M−H 410.3.

### (ii) (6S)-6-amino-4-methoxy-N-quinolin-3-yl-5,6,7,8-tetrahydronaphthalene-1-sulfonamide

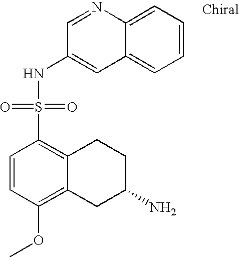

2,2,2-trifluoro-N-{(2S)-8-methoxy-5-[(quinolin-3-ylamino)sulfonyl]-1,2,3,4-tetrahydronaphthalen-2-yl}acetamide (170 mg, 0.355 mmol), ammonia in methanol (7M, 2 ml) and water (0.05 ml) were stirred in a sealed vial for 24 hours at 80° C. The solvent was removed under reduced to yield the title compound (170 mg, 0.355 mmol), which was used in subsequent reaction steps without further purification.

MS m/z M+H 384.4, M−H 382.4.

### (iii) 2,2,2-trifluoro-N-{(2S)-8-methoxy-5-[(quinolin-3-ylamino)sulfonyl]-1,2,3,4-tetrahydronaphthalen-2-yl}acetamide

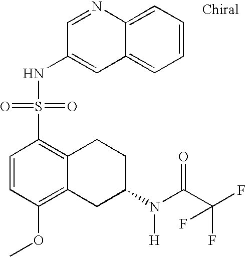

The method described in Example 42 (ii) was used to yield the title compound (98%).

¹H NMR (400 MHz, CDCl₃) δ ppm 8.56 (d, 1H) 7.99 (d, 1H) 7.95 (d, 1H) 7.92 (d, 1H) 7.71 (dd, 1H) 7.59-7.65 (m, 1H) 7.49-7.54 (m, 1H) 6.67 (d, 1H) 4.18-4.27 (m, 1H) 3.78 (s, 3H) 3.50-3.59 (m, 1H) 3.13-3.25 (m, 2H) 2.48 (dd, 1H) 2.08-2.16 (m, 1H) 1.75-1.87 (m, 1H) 1.72 (br. s., 1H)

MS m/z M+H 480.1, M−H 378.2.

### Example 54

### (i) (6S)-6-(dimethylamino)-N-isoquinolin-3-yl-4-methoxy-5,6,7,8-tetrahydronaphthalene-1-sulfonamide

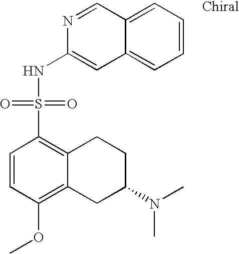

The method described in Example 31 (i) was used to give the title compound (97%).

¹H NMR (400 MHz, CDCl₃) δ ppm 8.60 (d, 1H) 7.98 (d, 1H) 7.89-7.94 (m, 2H) 7.69 (d, 1H) 7.56-7.62 (m, 1H) 7.46-7.52 (m, 1H) 6.64 (d, 1H) 3.79 (s, 3H) 3.58-3.68 (m, 1H) 2.97-3.05 (m, 1H) 2.94 (dd, 1H) 2.51-2.60 (m, 1H) 2.40-2.47 (m, 1H) 2.37 (s, 6H) 2.08-2.16 (m, 1H) 1.48-1.61 (m, 1H). MS m/z M+H 412.3. M−H 410.3.

### (ii) (6S)-6-amino-N-isoquinolin-3-yl-4-methoxy-5,6,7,8-tetrahydronaphthalene-1-sulfonamide

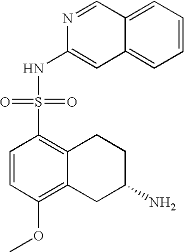

The method described in Example 53 (ii) was used to give the title compound (97%).

MS m/z M+H 382.4, M−H 382.4.

### (iii) 2,2,2-trifluoro-N-{(2S)-5-[(isoquinolin-3-ylamino)sulfonyl]-8-methoxy-1,2,3,4-tetrahydronaphthalen-2-yl}acetamide

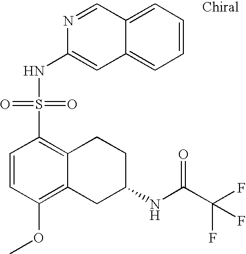

The method described in Example 42 (ii) was used to give the title compound (48%).

MS m/z M+H 480.2, M−H 478.3.

### Example 55

### (i) (6S)—N-1,3-benzothiazol-6-yl-6-(dimethylamino)-4-methoxy-5,6,7,8-tetrahydronaphthalene-1-sulfonamide

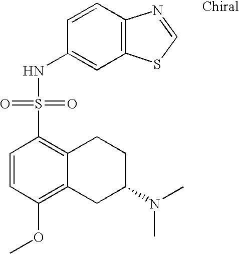

The method described in Example 31 (i) was used to give the title compound (45%).

¹H NMR (400 MHz, CDCl₃) δ ppm 8.90 (s, 1H) 7.83-7.99 (m, 2H) 7.68-7.76 (m, 1H) 7.15 (d, 1H) 6.67 (d, 1H) 3.79-3.87 (m, 3H) 2.88-3.04 (m, 2H) 2.39-2.57 (m, 2H) 2.34-2.37 (m, 6H) 2.07-2.18 (m, 1H) 1.48-1.62 (m, 1H) 1.41-1.46 (m, 1H).

MS m/z M+H 418.2, M−H 416.3.

### (ii) (6S)-6-amino-N-1,3-benzothiazol-6-yl-4-methoxy-5,6,7,8-tetrahydronaphthalene-1-sulfonamide

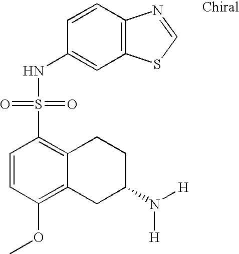

The method described in Example 53 (ii) was used to give the title compound (99%).

MS m/z M+H 390.3, M−H 388.4.

### (iii) N-{(2S)-5-[(1,3-benzothiazol-6-ylamino)sulfonyl]-8-methoxy-1,2,3,4-tetrahydronaphthalen-2-yl}-2,2,2-trifluoroacetamide

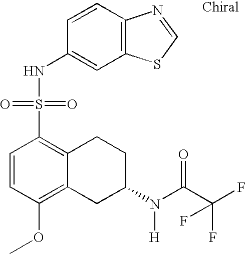

The method described in Example 42 (ii) was used to yield the title compound (92%).

¹H NMR (400 MHz, CDCl₃) δ ppm 8.90 (s, 1H) 7.93-7.98 (m, 2H) 7.72 (d, 1H) 7.13 (dd, 1H) 6.72 (d, 1H) 4.18-4.28 (m, 1H) 3.84 (s, 3H) 3.46-3.55 (m, 1H) 3.11-3.23 (m, 2H) 2.49 (dd, 1H) 2.10-2.19 (m, 1H) 1.75-1.87 (m, 1H). MS m/z M+H 485.7. M−H 483.7.

### Example 56

### (i) (2S)-5-[(3-chloro-1H-pyrrolo[2,3-b]pyridin-1-yl)sulfonyl]-8-methoxy-N,N-dimethyl-1,2,3,4-tetrahydronaphthalen-2-amine

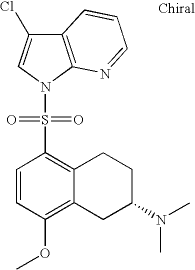

The method described in Example 31 (i) was used to give the title compound (45%).

¹H NMR (400 MHz, CDCl₃) δ ppm 8.37 (dd, 1H) 8.32 (d, 1H) 7.89 (dd, 1H) 7.81 (s, 1H) 7.23 (dd, 1H) 6.83 (d, 1H) 3.89 (s, 3H) 3.36-3.45 (m, 1H) 2.89-2.99 (m, 1H) 2.67-2.78 (m, 1H) 2.36-2.47 (m, 2H) 2.34 (s, 6H) 2.06-2.14 (m, 1H) 1.39-1.51 (m, 11H) MS m/z M+H 420.2, 422.1, M−H 418.2, 420.3.

### (ii) (2S)-5-[(3-chloro-1H-pyrrolo[2,3-b]pyridin-1-yl)sulfonyl]-8-methoxy-1,2,3,4-tetrahydronaphthalen-2-amine

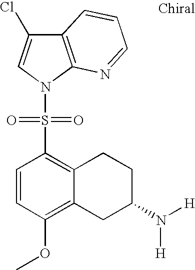

The method described in Example 53 (ii) was used to give the title compound (99%).

MS m/z M+H 392.2, 494.1.

### (iii) N-{(2S)-5-[(3-chloro-1H-pyrrolo[2,3-b]pyridin-1-yl)sulfonyl]-8-methoxy-1,2,3,4-tetrahydronaphthalen-2-yl}-2,2,2-trifluoroacetamide

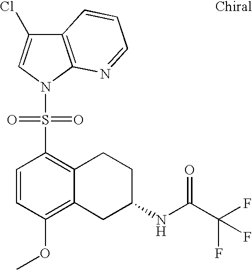

The method described in Example 52 (ii) was used to give the title compound (42%).

¹H NMR (400 MHz, CDCl₃) δ ppm 8.33-8.37 (m, 2H) 7.91 (dd, 1H) 7.79 (s, 1H) 7.25 (dd, 1H) 6.87 (d, 1H) 4.09-4.18 (m, 1H) 3.90 (s, 3H) 3.34-3.43 (m, 1H) 3.15 (dd, 1H) 2.83-2.94 (m, 1H) 2.43 (dd, 1H) 2.06-2.14 (m, 1H) 1.69-1.79 (m, 1H)

MS m/z M+H 488.2, 490.0, M−H 486.2, 488.2.

### Example 57

### (i) (2S)-5-(1H-benzimidazol-1-ylsulfonyl)-8-methoxy-N,N-dimethyl-1,2,3,4-tetrahydronaphthalen-2-amine

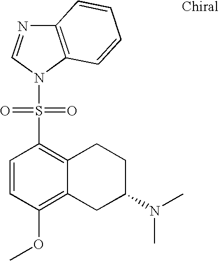

The method described in Example 31 (i) was used to give the title compound (13%).

¹H NMR (400 MHz, CDCl₃) δ ppm 8.46 (s, 1H) 8.16 (d, 1H) 7.80 (m, 1H) 7.54-7.58 (m, 1H) 7.28-7.38 (m, 2H) 6.85 (d, 1H) 3.91 (s, 3H) 3.35-3.44 (m, 1H) 2.88-2.95 (m, 1H) 2.51-2.63 (m, 1H) 2.33-2.45 (m, 2H) 2.32 (s, 6H) 2.03-2.11 (m, 1H) 1.39-1.51 (m, 1H).

MS m/z M+H 384.4, M−H 386.3.

### (ii) (2S)-5-(1H-benzimidazol-1-ylsulfonyl)-8-methoxy-1,2,3,4-tetrahydronaphthalen-2-amine

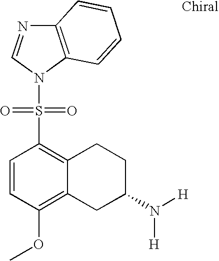

N-[(2S)-5-(1H-benzimidazol-1-ylsulfonyl)-8-methoxy-1,2,3,4-tetrahydronaphthalen-2-yl]-2,2,2-trifluoroacetamide (120 mg, 0.265 mmol), ammonia in methanol (7 M, 4 ml) and ammonia in water (33%, 1 ml) were heated under stirring for 1 day at 70° C. The solvent was removed under reduced pressure and the title compound (135 mg, 99%) was used in the subsequent reaction without further purification.

MS m/z M+H 358.1, M−H 356.2.

### (iii) N-[(2S)-5-(1H-benzimidazol-1-ylsulfonyl)-8-methoxy-1,2,3,4-tetrahydronaphthalen-2-yl]-2,2,2-trifluoroacetamide

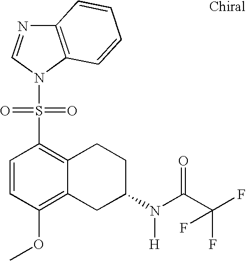

(6S)-4-Methoxy-6-[(trifluoroacetyl)amino]-5,6,7,8-tetrahydronaphthalene-1-sulfonyl chloride (99 mg, 0.266 mmol) and benzimidazole (94 mg, 0.799 mmol) were dissolved in dichloromethane (10 ml) and the reaction mixture was stirred for 10 hours. The mixture was diluted with dichloromethane and washed successively with water and sat. aqueous NaHCO₃ solution. The organic phase was dried (Na₂SO₄) and the solvent was removed under reduced pressure and the title compound was used without further purification in the subsequent reaction step.

MS m/z M+H 454.2, M−H 452.3.

### Example 58

### (i) (6S)—N-(4-cyanophenyl)-4-methoxy-6-(methylamino)-5,6,7,8-tetrahydronaphthalene-1-sulfonamide

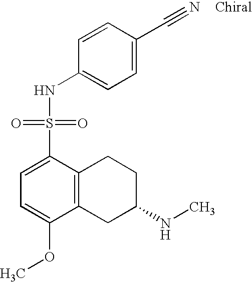

The method described in Example 57 (ii) was used to give the title compound (99%).

¹H NMR (400 MHz, DMSO-d₆) δ ppm 7.92 (d, 1H) 7.62 (d, 2H) 7.13 (d, 2H) 7.01 (d, 1H) 3.86 (s, 3H) 3.46-3.55 (m, 1H) 3.29-3.40 (m, 1H) 3.11 (dd, 1H) 2.83-2.95 (m, 1H) 2.63 (s, 3H) 2.42-2.50 (m, 1H) 2.18-2.26 (m, 1H) 1.57-1.69 (m, 1H). MS m/z M+H 372.2, M−H 370.3.

### (ii) N-((2S)-5-{[(4-cyanophenyl)amino]sulfonyl}-8-methoxy-1,2,3,4-tetrahydronaphthalen-2-yl)-2,2,2-trifluoro-N-methylacetamide

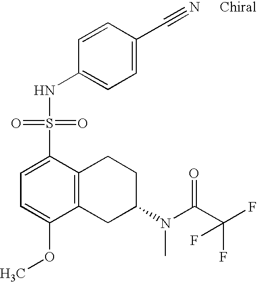

The method described in Example 29 (iii) was used to give the title compound (89%).

¹H NMR (400 MHz, CDCl₃) δ ppm 7.98-8.04 (m, 1H) 7.46-7.55 (m, 2H) 7.07-7.13 (m, 2H) 6.76-6.83 (m, 1H) 4.62-4.72 (m, 1H) 3.87-3.91 (m, 3H) 3.60-3.72 (m, 1H) 2.94-3.08 (m, 5H) 2.52-2.78 (m, 1H) 1.95-2.04 (m, 1H) 1.82-1.95 (m, 1H). MS m/z M+H 468.3, M+NH₄ 485.3, M−H 466.4.

### Example 59

### (i) (6S)-6-(methylamino)-N-[4-(trifluoromethyl)phenyl]-5,6,7,8-tetrahydronaphthalene-1-sulfonamide

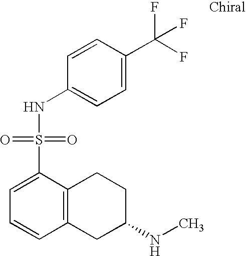

The method described in Example 57 (ii) was used to yield the title compound (46%).

¹H NMR (400 MHz, DMSO-d₆) δ ppm 7.72-7.79 (m, 1H) 7.30 (d, 2H) 7.18-7.23 (m, 2H) 6.96 (d, 2H) 3.52-3.62 (m, 1H) 3.04-3.13 (m, 2H) 2.91-3.03 (m, 1H) 2.62-2.72 (m, 1H) 2.48-2.50 (m, 3H) 2.06-2.15 (m, 1H) 1.49-1.61 (m, 1H). MS m/z M+H 385.2, M−H 383.2.

### (ii) 2,2,2-trifluoro-N-methyl-N-[(2S)-5-({[4-(trifluoromethyl)phenyl]amino}sulfonyl)-1,2,3,4-tetrahydronaphthalen-2-yl]acetamide

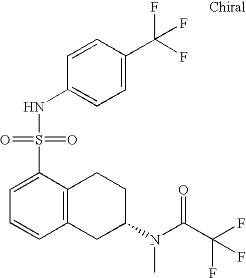

To a stirred solution of (7S)-7-[methyl(trifluoroacetyl)amino]-4-({[4-(trifluoromethyl)phenyl]amino}sulfonyl)-5,6,7,8-tetrahydronaphthalen-1-yl trifluoromethanesulfonate (120 mg, 0.191 mmol) in DMF (3 ml) under argon atmosphere were sequentially added triethylamine (0.2 ml, 1.5 mmol), formic acid (0.06 ml, 1.5 mmol), triphenylphosphine (20 mg, 0.08 mmol) and palladium diacetate (5 mg, 0.02 mmol). The mixture was heated to 80° C. for 10 hours. After cooling to RT the mixture was diluted with diethyl ether (40 ml) and dichloromethane (5 ml), washed successively with hydrochloric acid (1 M), water, aqueous K₂CO₃ (0.5 M) and water. The organic phase was dried (Na₂SO₄) and the solvent was removed under reduced pressure. The residue was purified by chromatography on silica using a gradient of heptane/ethyl acetate reaching from 0-100% of ethyl acetate as eluent to give the product (63 mg, 68%).

MS m/z M+H 481.2, M−H 479.2.

### (iii) (7S)-7-[methyl(trifluoroacetyl)amino]-4-({[4-(trifluoromethyl)phenyl]amino}sulfonyl)-5,6,7,8-tetrahydronaphthalen-1-yl trifluoromethanesulfonate

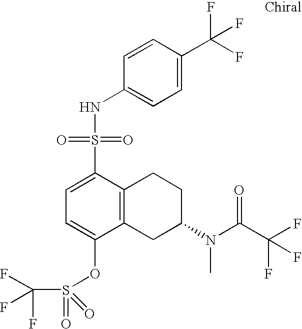

A solution of 2,2,2-trifluoro-N-[(2S)-8-hydroxy-5-({[4-(trifluoromethyl)phenyl]amino}sulfonyl)-1,2,3,4-tetrahydronaphthalen-2-yl]-N-methylacetamide (348 mg, 0.7 mmol) and triethyl amine (0.147 ml, 1.05 mmol) in dichloromethane (4 ml) was cooled to −10° C. and trifluoroacetic anhydride (0.128 ml, 0.77 mmol) was added dropwise. The mixture was stirred at −10° C. for 15 min and then kept at 0° C. for 72 hours. The reaction mixture was poured on water, the phases were separated and the organic phase was successively washed with hydrochloric acid (1 M), water and sat. aqueous NaHCO₃. The organic phase was dried (Na₂SO₄) and the solvent was removed under reduced pressure. The residue was purified by chromatography on silica using a gradient of heptane/ethyl acetate reaching from 0-100% of ethyl acetate as eluent to give the title compound (133 mg, 30%).

¹H NMR (400 MHz, CDCl₃) δ ppm 8.03 (d, 1H) 7.51 (d, 2H) 7.29 (d, 1H) 7.17 (d, 2H) 4.65-4.75 (m, 1H) 3.73-3.81 (m, 1H) 3.11-3.20 (m, 1H) 3.00-3.11 (m, 4H) 2.82 (dd, 1H) 2.04-2.13 (m, 1H) 1.91-2.03 (m, 1H). MS m/z M+N 646.2, M−H 627.3.

### (iv) 2,2,2-trifluoro-N-[(2S)-8-hydroxy-5-({[4-(trifluoromethyl)phenyl]amino}sulfonyl)-1,2,3,4-tetrahydronaphthalen-2-yl]-N-methylacetamide

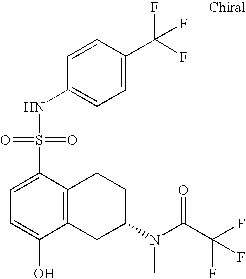

A solution of 2,2,2-trifluoro-N-[(2S)-8-methoxy-5-({[4-(trifluoromethyl)phenyl]amino}sulfonyl)-1,2,3,4-tetrahydronaphthalen-2-yl]-N-methylacetamide (350 mg, 0.686 mmol) in dichloromethane (5 ml) was cooled to 0° C. BBr₃ (0.324 ml, 3.43 mmol) was added and the reaction mixture was stirred for one hour allowing the reaction mixture to reach RT. Ice was added and the mixture was extracted with dichloromethane. The organic phase was washed with sat. aqueous NaHCO₃ solution and dried (Na₂SO₄). The solvent was removed under reduced pressure and the residue was purified by chromatography on silica, using dichloromethane/methanol gradient mixtures (0-20% methanol) as eluent to give the title compound (238 mg, 60%).

MS m/z M+H 497.3, M−H 495.3.

### (v) 2,2,2-trifluoro-N-[(2S)-8-methoxy-5-({[4-(trifluoromethyl)phenyl]amino}sulfonyl)-1,2,3,4-tetrahydronaphthalen-2-yl]-N-methylacetamide

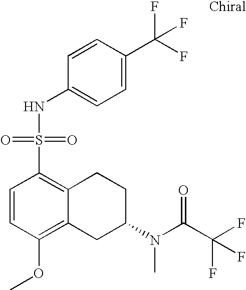

The method described in Example 29 (iii) was used to yield the title compound (99%).

MS m/z M+H 511.0, M−H 509.1.

### Example 60

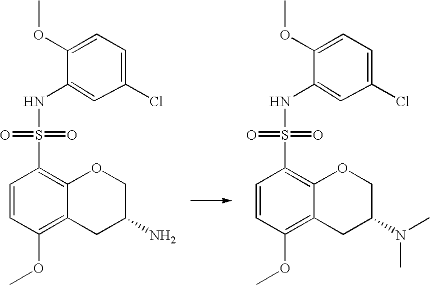

### (i) (3R)—N-(5-Chloro-2-methoxyphenyl)-3-(dimethylamino)-5-methoxychromane-8-sulfonamide

Formaldehyde (37% aqueous, 100 μl, 1.2 mmol) was added to a slurry of (3R)-3-Amino-N-(5-chloro-2-methoxyphenyl)-5-methoxychromane-8-sulfonamide (60 mg, 0.15 mmol) in methanol (1 ml). The reaction was stirred for 10 minutes followed by the portion wise addition of sodium cyanoborohydride (76 mg, 1.2 mmol). Acetic acid (1 drop) was added to the mixture and the reaction stirred at ambient temperature overnight. The solvent was evaporated, ethyl acetate and a solution of sodium bicarbonate was added and the phases were separated. The organic phase was dried (Na₂SO₄), filtered and evaporated to dryness. The crude was purified by preparative HPLC to give the title compound as a solid (44 mg, 69%). ¹H NMR (400 MHz, CDCl₃) δ ppm 7.75 (d, 1H) 7.57 (s, 1H) 7.52 (d, 1H) 6.90 (dd, 1H) 6.68 (d, 1H) 6.46 (d, 1H) 4.51-4.58 (m, 1H) 3.85 (s, 3H) 3.82 (s, 3H) 3.71-3.78 (m, 1H) 2.85-2.96 (m, 1H) 2.67 (s, 1H) 2.40-2.50 (bs, 1H) 2.40 (s, 6H); MS m/z M+H 427, M−H 425.

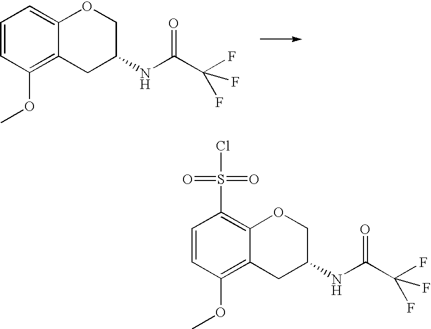

### (ii) (3R)-5-Methoxy-3-[(trifluoroacetyl)amino]chromane-8-sulfonyl chloride

(3R)-5-Methoxy-3-[(trifluoroacetyl)amino]chromane (1.0 g, 3.4 mmol) was dissolved in anhydrous chloroform (13 ml), cooled to −15° C. and thionyl chloride (795 μl, 10.2 mmol) was added. A solution of chlorosulfonic acid (490 μl, 6.8 mmol) in chloroform (13 ml) added dropwise under 10 minutes and the mixture was allowed to come to room temperature. Dimethylformamide (50 drops) was added to give a homogeneous clear solution and the reaction stirred for 3 hours at room temperature.

The reaction was poured out on ice, extracted with chloroform (×3), washed with a sodium bicarbonate solution, dried (MgSO₄), and evaporated to give the title compound as an oil (1.6 g, 90%). ¹H NMR (600 MHz, CDCl₃) δ ppm 7.88 (d, 1H) 6.75 (d, 1H) 6.61 (d, 1H) 4.58-4.71 (m, 1H) 4.44-4.55 (m, 1H) 4.33 (dd, 1H) 3.95 (s, 3H) 3.02 (dd, 1H) 2.84-2.89 (m, 1H); MS m/z M−H 372.

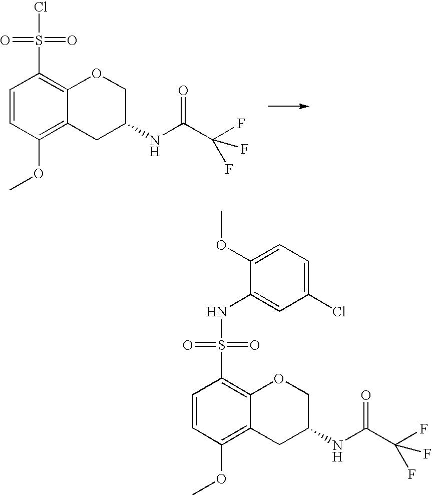

### (iii) N-((3R)-8-{[(5-Chloro-2-methoxyphenyl)amino]sulfonyl}-5-methoxy-3,4-dihydro-2H-chromen-3-yl)-2,2,2-trifluoroacetamide

To a solution of 5-chloro-2-methoxyaniline (270 mg, 1.72 mmol) and pyridine (255 μl, 3.3 mmol) in anhydrous chloroform (6 ml) was a solution of (3R)-5-methoxy-3-[(trifluoroacetyl)amino]chromane-8-sulfonyl chloride (535 mg, 1.43 mmol) in chloroform (3 ml) added dropwise and the reaction stirred at room temperature overnight.

The solvent was evaporated and ethyl acetate and saturated ammonium chloride solution was added. The phases were separated and the aqueous phase was extracted with ethyl acetate, dried (MgSO₄), filtered, and the solvent was removed by evaporation. The crude product was purified on silica (ethyl acetate/hexane 2:3) to give the title compound (430 mg, 61%). ¹H NMR (400 MHz, DMSO-d₆) δ ppm 9.57 (d, 1H) 8.70 (s, 1H) 7.57 (d, 1H) 7.28 (d, 1H) 7.08 (dd, 1H) 6.92-7.02 (m, 1H) 6.66 (d, 1H) 4.19 (d, 2H) 3.92-3.99 (m, 1H) 3.82 (s, 3H) 3.73 (s, 3H) 2.92 (dd, 1H) 2.62 (dd, 1H); MS m/z M+H 495, M−H 493.

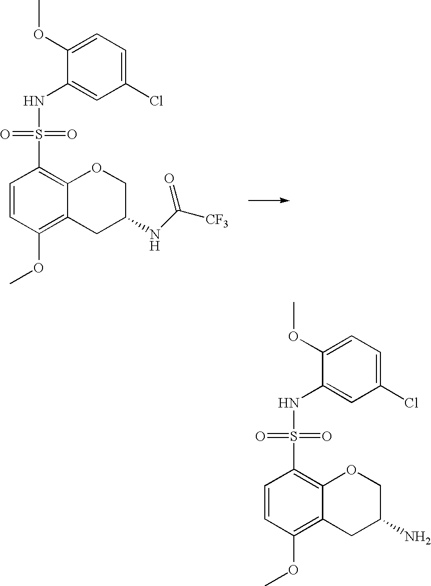

### (iv) (3R)-3-Amino-N-(5-chloro-2-methoxyphenyl)-5-methoxychromane-8-sulfonamide

N-((3R)-8-{[(5-Chloro-2-methoxyphenyl)amino]sulfonyl}-5-methoxy-3,4-dihydro-2H-chromen-3-yl)-2,2,2-trifluoroacetamide (430 mg, 0.87 mmol) was dissolved in chloroform (4 ml) and a 2 M sodium hydroxide solution (4 ml) was added. The reaction was allowed to stir at room temperature for 1 hour. The mixture was diluted with chloroform and water, the mixture was acidified with concentrated hydrochloric acid, made basic with a sodium bicarbonate solution. The mixture was stirred vigorously until the solid went into solution. The mixture was extracted with chloroform (×3), dried (Na₂SO₄) and the solvent was evaporated to give the title compound as a solid (345 mg, 100%). ¹H NMR (400 MHz, DMSO-d₆) δ ppm 8.31 (s, 1H) 7.53 (d, 1H) 7.28 (d, 1H) 7.02-7.12 (m, 1H) 6.96 (d, 1H) 6.60 (d, 1H) 4.09-4.25 (m, 1H) 3.93-4.08 (m, 2H) 3.80 (s, 3H) 3.73 (s, 3H) 3.51-3.64 (m, 1H) 3.02-3.15 (m, 1H) 2.78 (dd, 1H) 2.18 (dd, 1H); MS m/z M+H 399, M−H 397.

### Example 61

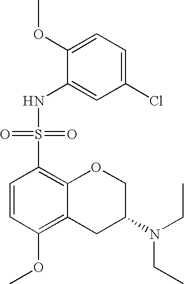

### (i) (3R)—N-(5-Chloro-2-methoxyphenyl)-3-(diethylamino)-5-methoxychromane-8-sulfonamide

The title compound was synthesized by the analogous preparation of Example 60 (i) and was obtained as a solid (34 mg, 60%). ¹H NMR (400 MHz, CDCl₃) δ ppm 7.75 (d, 1H) 7.58 (s, 1H) 7.52 (d, 1H) 6.89 (dd, 1H) 6.68 (d, 1H) 6.45 (d, 1H) 4.44-4.54 (m, 1H) 3.85 (s, 3H) 3.81 (s, 3H) 3.72 (t, 1H) 3.06-3.18 (m, 1H) 2.81-2.91 (m, 1H) 2.67 (q, 4H) 2.45 (dd, 1H) 1.06 (t, 6H); MS m/z M+H 455, M−H 453.

### Example 62

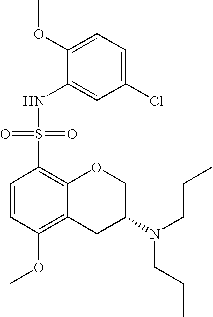

### (i) (3R)—N-(5-Chloro-2-methoxyphenyl)-3-(dipropylamino)-5-methoxychromane-8-sulfonamide

The title compound was synthesized by the analogous preparation of Example 60 (i) and was obtained as a solid (38 mg, 63%). ¹H NMR (400 MHz, CDCl₃) δ ppm 7.74 (d, 1H) 7.58 (s, 1H) 7.52 (d, 1H) 6.90 (dd, 1H) 6.69 (d, 1H) 6.45 (d, 1H) 4.45 (d, 1H) 3.85 (s, 3H) 3.81 (s, 3H) 3.70 (t, 1H) 3.03-3.18 (m, 1H) 2.81 (dd, 1H) 2.36-2.60 (m, 5H) 1.37-1.53 (m, 4H) 0.89 (t, 6H); MS m/z M+H 483, M−H 481.

### Example 63

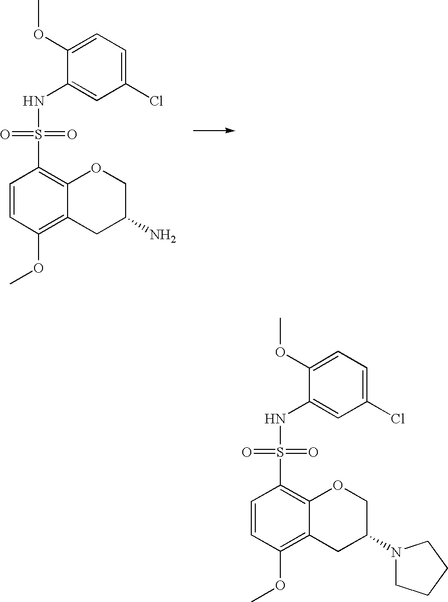

### (i) (3R)—N-(5-Chloro-2-methoxyphenyl)-5-methoxy-3-pyrrolidin-1-ylchromane-8-sulfonamide

(3R)-3-Amino-N-(5-chloro-2-methoxyphenyl)-5-methoxychromane-8-sulfonamide (123 mg, 0.31 mmol) was slurried in toluene (5 ml) and to the mixture were 1,4-dibromobutane (82 μl, 0.62 mmol), sodium bicarbonate (80 mg, 1.0 mmol) and a crystal of potassium iodide added. The reaction was refluxed for 1 week. The reaction was filtered, evaporated and purified by preparative HPLC to give the title compound as a solid (36 mg, 36%). ¹H NMR (400 MHz, CDCl₃) δ ppm 7.75 (d, 1H) 7.57 (s, 1H) 7.53 (d, 1H) 6.90 (dd, 1H) 6.68 (d, 1H) 6.45 (d, 1H) 4.58 (dd, 1H) 3.85 (s, 3H) 3.81 (s, 3H) 3.73 (s, 1H) 2.90-3.04 (m, 1H) 2.70 (s, 4H) 2.57-2.64 (m, 1H) 2.42 (s, 1H) 1.84 (s, 4H); MS m/z M+H 453, M−H 451.

### Example 64

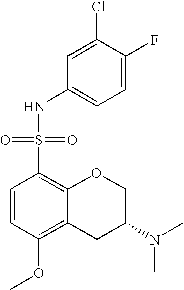

### (i) (3R)—N-(3-Chloro-4-fluorophenyl)-3-(dimethylamino)-5-methoxychromane-8-sulfonamide

The title compound was synthesized by the analogous preparation of Example 60 (i) and was obtained as a solid (38 mg, 88%). ¹H NMR (400 MHz, CD₃OD) δ ppm 7.61 (d, 1H) 7.21-7.27 (m, 1H) 7.03-7.08 (m, 2H) 6.59 (d, 1H) 4.47 (dd, 1H) 3.97 (dd, 1H) 3.86 (s, 3H) 2.89 (dd, 1H) 2.66-2.75 (m, 1H) 2.56 (dd, 1H) 2.39 (s, 6H); MS m/z M+H 415, M−H 413.

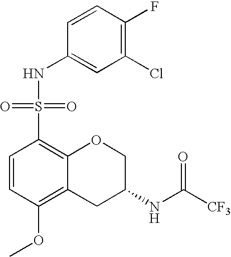

### (ii) N-((3R)-8-{[(3-Chloro-4-fluorophenyl)amino]sulfonyl}-5-ethoxy-3,4-dihydro-2H-chromen-3-yl)-2,2,2-trifluoroacetamide

The title compound was synthesized by the analogous preparation of Example 60 (iii) and was obtained as a solid (345 mg, 99%). ¹H NMR (400 MHz, CDCl₃) δ ppm 7.52 (d, 1H) 7.04 (dd, 1H) 6.69-6.83 (m, 2H) 6.51 (d, 1H) 6.32 (d, 1H) 4.37 (s, 1H) 4.23-4.33 (m, 1H) 4.06 (dd, 1H) 3.67 (s, 3H) 2.70-2.87 (m, 1H) 2.50-2.66 (m, 1H); MS m/z M+H 483, M−H 481.

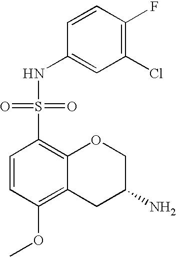

### (iii) (3R)-3-Amino-N-(3-chloro-4-fluorophenyl)-5-methoxychromane-8-sulfonamide

The title compound was synthesized by the analogous preparation Example 60 (iv) and was obtained as a solid (254 mg, 92%). ¹H NMR (400 MHz, CDCl₃) δ ppm 7.63 (d, 1H) 7.22 (dd, 1H) 6.99-7.07 (m, 1H) 6.97 (t, 1H) 6.44 (d, 1H) 4.30-4.38 (m, 1H) 3.98 (dd, 1H) 3.84 (s, 3H) 3.38-3.46 (m, 1H) 2.95 (dd, 1H) 2.41 (dd, 1H) 1.30-1.68 (m, 2H); MS m/z M+H 387, M−H 385.

### Example 65

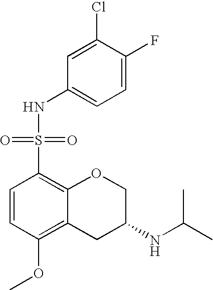

### (3R)—N-(3-Chloro-4-fluorophenyl)-3-(isopropylamino)-5-methoxychromane-8-sulfonamide

The title compound was synthesized by the analogous preparation of Example 60 (i) and is was obtained as an oil (24 mg, 67%). ¹H NMR (400 MHz, CD₃OD) δ ppm 7.63 (dd, 1H) 7.23 (d, 1H) 7.00-7.10 (m, 2H) 6.59 (dd, 1H) 4.36 (d, 1H) 3.75-3.91 (m, 4H) 3.13-3.23 (m, 1H) 3.01-3.11 (m, 1H) 2.92 (dd, 1H) 2.32-2.45 (m, 1H) 1.04-1.17 (m, 6H); MS m/z M+H 429, M−H 427.

### Example 66

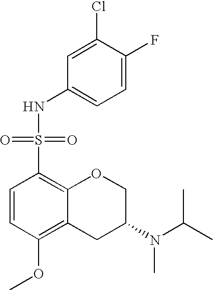

### (3R)—N-(3-Chloro-4-fluorophenyl)-3-[isopropyl(methyl)amino]-5-methoxychromane-8-sulfonamide

The title compound was synthesized by the analogous preparation of Example 60 (i) and was obtained as a solid (35 mg, 94%). ¹H NMR (400 MHz, CD₃OD) δ ppm 7.62 (d, 1H) 7.20-7.26 (m, 1H) 7.01-7.09 (m, 2H) 6.60 (d, 1H) 4.46-4.54 (m, 1H) 3.92 (dd, 1H) 3.86 (s, 3H) 3.17-3.27 (m, 1H) 3.04-3.15 (m, 1H) 2.83-2.94 (m, 1H) 2.59 (dd, 1H) 2.33 (s, 3H) 1.12 (dd, 6H); MS m/z M+H 443, M−H 441.

### Example 67

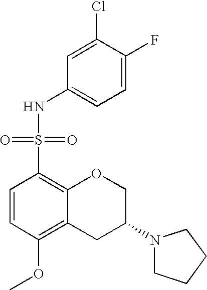

### (3R)—N-(3-Chloro-4-fluorophenyl)-5-methoxy-3-pyrrolidin-1-ylchromane-8-sulfonamide

The title compound was synthesized by the analogous preparation of Example 63 (i) and was obtained as a solid (100 mg, 66%). ¹H NMR (400 MHz, CD₃OD) δ ppm 7.61 (d, 1H) 7.23 (d, 1H) 6.97-7.13 (m, 2H) 6.50-6.65 (m, 1H) 4.51 (d, 1H) 3.87-4.00 (m, 1H) 3.85 (s, 3H) 2.87-3.00 (m, 1H) 2.74 (s, 4H) 2.61-2.69 (m, 1H) 2.52 (dd, 1H) 1.78-1.90 (m, 4H); MS m/z M+H 441, M−H 439.

### Example 68

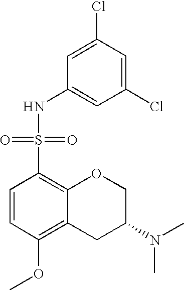

### (i) (3R)—N-(3,5-Dichlorophenyl)-3-(dimethylamino)-5-methoxychromane-8-sulfonamide

The title compound was synthesized by the analogous preparation of Example 60 (i) and was obtained as a solid (37 mg, 86%). ¹H NMR (400 MHz, CD₃OD) δ ppm 7.70 (d, 1H) 7.08 (d, 2H) 7.02 (t, 1H) 6.64 (d, 1H) 4.40-4.47 (m, 1H) 3.96 (dd, 1H) 3.88 (s, 3H) 2.88 (dd, 1H) 2.65-2.74 (m, 1H) 2.56 (dd, 1H) 2.37 (s, 6H); MS m/z M+H 431, M−H 429.

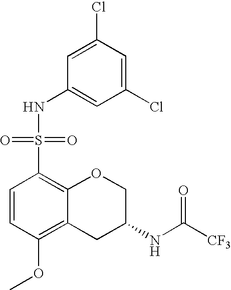

### (ii) N-((3R)-8-{[(3,5-Dichlorophenyl)amino]sulfonyl}-5-methoxy-3,4-dihydro-2H-chromen-3-yl)-2,2,2-trifluoroacetamide

The title compound was synthesized by the analogous preparation of Example 60 (iii) and was obtained as an oil (170 mg, 47%). ¹H NMR (600 MHz, CDCl₃) δ ppm 7.80 (d, 1H) 7.72 (d, 1H) 7.03 (d, 2H) 7.01 (t, 1H) 6.72-6.78 (m, 1H) 6.55 (d, 1H) 4.54 (s, 1H) 4.41-4.46 (m, 1H) 4.22 (dd, 1H) 3.87 (s, 3H) 2.96 (dd, 1H) 2.78 (dd, 1H); MS m/z M+H 499, M−H 497.

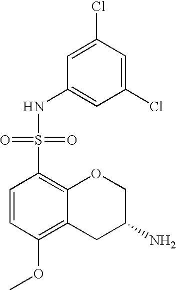

### (iii) (3R)-3-Amino-N-(3,5-dichlorophenyl)-5-methoxychromane-8-sulfonamide

The title compound was synthesized by the analogous preparation of Example 60 (iv) and was obtained as a white solid (117 mg, 85%). ¹H NMR (400 MHz, CDCl₃) δ ppm 7.72 (d, 1H) 7.05 (d, 2H) 7.02 (t, 1H) 6.48 (d, 1H) 4.28-4.36 (m, 1H) 3.96 (dd, 1H) 3.86 (s, 3H) 3.35-3.48 (m, 1H) 2.93 (dd, 1H) 2.40 (dd, 1H) 1.55 (bs, 2H); MS m/z M+H 403, M−H 401.

### Example 69

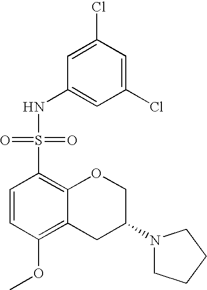

### (i) (3R)—N-(3,5-Dichlorophenyl)-5-methoxy-3-pyrrolidin-1-ylchromane-8-sulfonamide

The title compound was synthesized by the analogous preparation of Example 63 (i) and was obtained as solid (52 mg, 66%). ¹H NMR (400 MHz, CD₃OD) δ ppm 7.70 (d, 1H) 7.07 (d, 2H) 7.01 (t, 1H) 6.64 (d, 1H) 4.43-4.53 (m, 1H) 3.87 (s, 3H) 3.86 (dd, 1H) 2.89-2.99 (m, 1H) 2.72 (d, 4H) 2.61-2.69 (m, 1H) 2.51 (dd, 1H) 1.77-1.87 (m, 4H); MS m/z M+H 457, M−H 455.

### Example 70

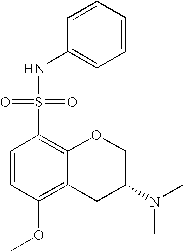

### (i) (3R)-3-(Dimethylamino)-5-methoxy-N-phenylchromane-8-sulfonamide

The title compound was synthesized by the analogous preparation of Example 60 (i) and was obtained as a solid (14 mg, 34%). ¹H NMR (400 MHz, CD₃OD) δ ppm 7.61 (d, 1H) 7.13-7.19 (m, 2H) 7.08-7.13 (m, 2H) 6.94-7.01 (m, 1H) 6.56 (d, 1H) 4.45-4.52 (m, 1H) 3.93 (dd, 1H) 3.84 (s, 3H) 2.85-2.94 (m, 1H) 2.68-2.78 (m, 1H) 2.52 (dd, 1H) 2.39 (s, 6H); MS m/z M+H 363, M−H 361.

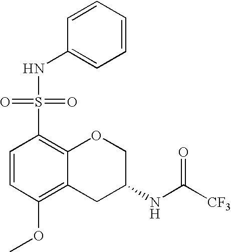

### (ii) N-[(3R)-8-(Anilinosulfonyl)-5-methoxy-3,4-dihydro-2H-chromen-3-yl]-2,2,2-trifluoroacetamide

The title compound was synthesized by the analogous preparation of Example 60 (iii) and was obtained as an oil (319 mg, 77%). ¹H NMR (400 MHz, CDCl₃) δ ppm 7.76 (d, 1H) 7.18-7.25 (m, 2H) 7.03-7.11 (m, 3H) 6.89 (s, 1H) 6.49 (d, 1H) 6.46 (d, 1H) 4.53-4.63 (m, 1H) 4.40-4.48 (m, 1H) 4.23 (dd, 1H) 3.84 (s, 3H) 2.95 (dd, 1H) 2.71-2.82 (m, 1H); MS m/z M+H 431, M−H 429.

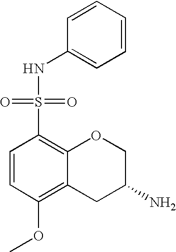

### (iii) (3R)-3-Amino-5-methoxy-N-phenylchromane-8-sulfonamide

The title compound was synthesized by the analogous preparation of Example 60 (iv) and was obtained as a white solid (247 mg, 99%). ¹H NMR (400 MHz, CDCl₃) δ ppm 7.66 (d, 1H) 7.17-7.25 (m, 2H) 7.10 (d, 2H) 7.05 (t, 1H) 6.89 (s, 1H) 6.42 (d, 1H) 4.28-4.37 (m, 1H) 3.95 (dd, 1H) 3.82 (s, 3H) 3.35-3.45 (m, 1H) 2.93 (dd, 1H) 2.38 (dd, 1H) 1.50 (bs, 2H); MS m/z M+H 335, M−H 333.

### Example 71

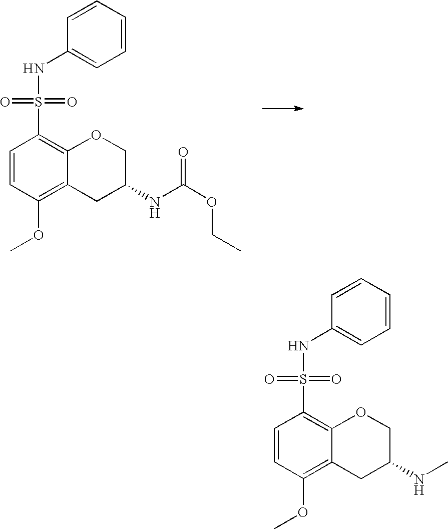

### (i) (3R)-5-Methoxy-3-(methylamino)-N-phenylchromane-8-sulfonamide

Ethyl [(3R)-8-(anilinosulfonyl)-5-methoxy-3,4-dihydro-2H-chromen-3-yl]carbamate (180 mg, 0.44 mmol) in tetrahydrofuran (4 ml) was added dropwise to a suspension of lithium aluminum hydride (51 mg, 1.3 mmol) in anhydrous tetrahydrofuran (1 ml). The reaction stirred for 30 minutes at room temperature and was then heated to reflux for 10 minutes. The reaction was carefully quenched with saturated sodium sulfate (255 μl), the reaction mixture was filtered, washed with tetrahydrofuran and the filtrate was evaporated. The remains were purified by preparative HPLC to give the title compound as an oil (27 mg, 18%). ¹H NMR (400 MHz, CDCl₃) δ ppm 7.67 (d, 1H) 7.20 (t, 2H) 7.11 (d, 2H) 7.04 (t, H) 6.42 (d, 1H) 4.33-4.40 (m, 1H) 4.18 (dd, 1H) 3.82 (s, 3H) 3.13-3.21 (m, 1H) 2.88 (dd, 1H) 2.51-2.61 (m, 1H) 2.56 (s, 3H); MS m/z M+H 349, M−H 347.

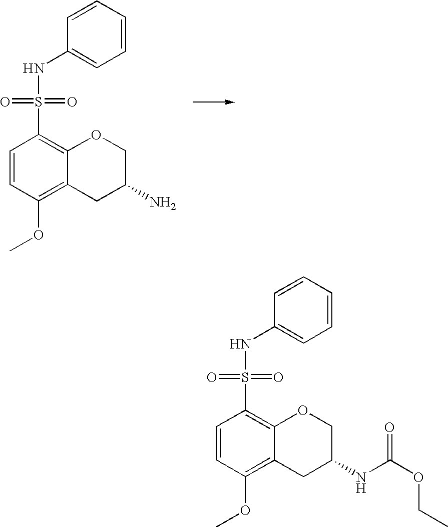

### (ii) Ethyl [(3R)-8-(Anilinosulfonyl)-5-methoxy-3,4-dihydro-2H-chromen-3-yl]carbamate

(3R)-3-Amino-5-methoxy-N-phenylchromane-8-sulfonamide Example 70 (iii) (208 mg, 0.62 mmol) was suspended in anhydrous dichloromethane (5 ml) and triethylamine (150 is 1, 1.05 mmol) and ethyl chloroformate (64 μl, 0.64 mmol) were added. The reaction was stirred for 15 minutes at room temperature. The solvent was evaporated, ethyl acetate and a sodium bicarbonate solution were added, the mixture was extracted with ethyl acetate (×2), dried (MgSO₄), filtered and evaporated. The remains were purified on silica (55% ethyl acetate/hexane) to give the title compound as an oil (180 mg, 71%). ¹H NMR (400 MHz, CDCl₃) δ ppm 7.69 (d, 1H) 7.31 (s, 1H) 7.20 (t, 2H) 7.09-7.15 (m, 2H) 7.03 (t, 1H) 6.41 (d, 1H) 4.92 (d, 1H) 4.37 (d, 1H) 4.18-4.30 (m, 2H) 4.06-4.17 (m, 2H) 3.79 (s, 3H) 2.83 (dd, 1H) 2.61-2.72 (m, 1H) 1.19-1.30 (m, 3H); MS m/z M+H 407, M−H 405.

### Example 72

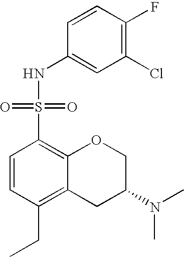

### (i) (3R)—N-(3-Chloro-4-fluorophenyl)-3-(dimethylamino)-5-ethylchromane-8-sulfonamide

The title compound was synthesized by the analogous preparation of Example 60 (i) and was obtained as an oil (40 mg, 62%). ¹H NMR (400 MHz, CD₃OD) δ ppm 7.55 (d, 1H) 7.24 (dd, 1H) 7.01-7.10 (m, 2H) 6.85 (d, 1H) 4.44-4.50 (m, 1H) 4.01 (dd, 1H) 2.96 (dd, 1H) 2.68-2.82 (m, 2H) 2.62 (q, 2H) 2.40 (s, 6H) 1.19 (t, 3H); MS m/z M+H 413, M−H 411.

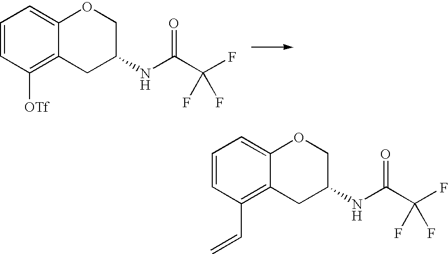

### (ii) 2,2,2-Trifluoro-N-[(3R)-5-vinyl-3,4-dihydro-2H-chromen-3-yl]acetamide

(3R)-3-[(2,2,2-trifluoroacetyl)amino]-3,4-dihydro-2H-chromen-5-yl trifluoromethanesulfonate (5.0 g, 12.7 mmol) was dissolved in dimethylformamide (80 ml) and the following were added in order under argon atmosphere: lithium chloride (1.62 g, 38.1 mmol), tributyl(vinyl)tin (4.09 ml, 14 mmol) and bis(triphenylphosphine)palladium (II) chloride (0.89 g, 1.27 mmol). The mixture was subjected to vacuum/argon (3 cycles) and was put in a pre-heated oil bath at 90° C. with stirring for 2 hours. The cooled reaction mixture was added to an ice/water mixture and was extracted with ethyl acetate (×2), washed with water and brine, dried (MgSO₄), filtered, and evaporated to dryness. The crude product was purified on silica (10-12.5% ethyl acetate in hexane) to give the title compound as a solid (2.9 g, 84%). ¹H NMR (400 MHz, CDCl₃) δ ppm 7.14-7.21 (m, 2H) 6.82-6.88 (m, 1H) 6.77 (dd, 1H) 6.67 (s, 1H) 5.68 (dd, 1H) 5.37 (dd, 1H) 4.55-4.65 (m, 1H) 4.19-4.26 (m, 1H) 4.10 (dd, 1H) 3.14 (dd, 1H) 2.83-2.92 (m, 1H); MS m/z M−H 270.

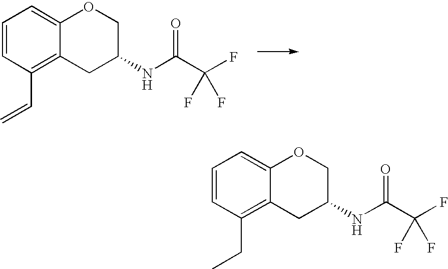

### (iii) N-[(3R)-5-Ethyl-3,4-dihydro-2H-chromen-3-yl]-2,2,2-trifluoroacetamide

2,2,2-Trifluoro-N-[(3R)-5-vinyl-3,4-dihydro-2H-chromen-3-yl]acetamide was dissolved in methanol (20 ml) and 10% palladium on carbon (300 mg) was added under nitrogen. The reaction mixture was subjected to hydrogen atmosphere for 30 minutes at room temperature.

The reaction mixture was filtered through diatomaceous earth and the solvent was evaporated to give the title compound as a solid (2.65 g, 95%). ¹H NMR (400 M, CDCl₃) δ ppm 7.14 (t, 1H) 6.87 (d, 1H) 6.78 (d, 1H) 6.68 (s, 1H) 4.55-4.65 (m, 1H) 4.18-4.26 (m, 1H) 4.10 (d, 1H) 3.08 (dd, 1H) 2.83 (d, 1H) 2.56 (q, 2H) 1.21 (t, 3H); MS m/z M−H 272.

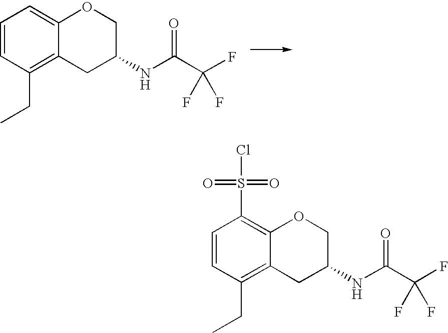

### (iv) (3R)-5-Ethyl-3-[(trifluoroacetyl)amino]chromane-8-sulfonyl chloride

N-[(3R)-5-Ethyl-3,4-dihydro-2H-chromen-3-yl]-2,2,2-trifluoroacetamide (0.4 g, 1.45 mmol) was dissolved in anhydrous chloroform (5 ml) and dimethylformamide (20 drops), cooled to −15° C. and thionyl chloride (215 μl, 2.9 mmol) was added. A solution of chlorosulfonic acid (295 μl, 3.8 mmol) in chloroform (5 ml) was added dropwise under 13 minutes to the stirred mixture, which was then allowed to come to room temperature. The reaction stirred for 5 days at room temperature. The reaction was poured on ice, extracted with chloroform (×3), washed with sodium bicarbonate solution, dried (MgSO₄), and evaporated to give the title compound (505 mg, 93%) as an oil. The title compound was the minor product (43% pure) in the mixture with the major being the regioisomer with the sulfonyl chloride in the 6 position. The crude was used without further purification, MS m/z M−H 370.

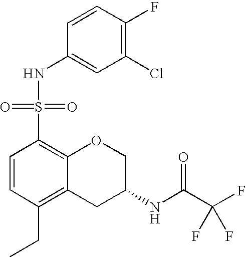

### (iv) N-((3R)-8-{[(3-Chloro-4-fluorophenyl)amino]sulfonyl}-5-ethyl-3,4-dihydro-2H-chromen-3-yl)-2,2,2-trifluoroacetamide

The title compound was synthesized by the analogous preparation of Example 60 (iii) and was obtained as an oil (145 mg, 22%). The product contained 40% of the regioisomer with the sulfonamide in the 6 position; MS m/z M+H 481, M−H 479.

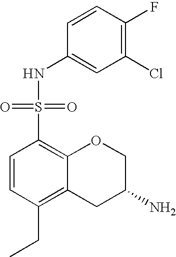

### (v) (3R)-3-Amino-N-(3-chloro-4-fluorophenyl)-5-ethylchromane-8-sulfonamide

The title compound was synthesized by the analogous preparation of Example 60 (iv) and was obtained as an oil (60 mg, 52%). ¹H NMR (400 MHz, CDCl₃) δ ppm 7.60 (d, 1H) 7.22 (dd, 1H) 7.00-7.06 (m, 1H) 6.96 (t, 1H) 6.82 (d, 1H) 4.39 (dd, 1H) 3.98 (dd, 1H) 3.42-3.51 (m, 1H) 3.00 (dd, 1H) 2.56 (q, 2H) 2.48 (dd, 1H) 1.19 (t, 3H); MS m/z M+H 385, M−H 383.

### Example 73

### (i) (3R)-6-Chloro-N-phenyl-3-pyrrolidin-1-ylchromane-8-sulfonamide

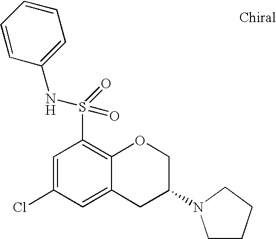

(3R)-3-Amino-6-chloro-N-phenylchromane-8-sulfonamide (40 mg, 0.12 mmol) was dissolved in toluene. 1,4-Dibromobutane (28 μl, 0.24 mmol), DIPEA (60 μl, 0.35 mmol) and a few crystals of potassium iodide were added. The mixture was heated to reflux for 16 hours. The solvent was evaporated and the residue was purified by preparative HPLC to give a solid (6 mg, 13%). ¹H NMR (400 MHz, DMSO-d₆) δ ppm 10.00 (s, 1H) 7.31-7.47 (m, 2H) 7.19 (t, 2H) 7.09 (d, 2H) 6.99 (t, 1H) 4.25-4.38 (m, 1H) 4.13-4.25 (m, 1H) 3.00 (dd, 1H) 2.82 (dd, 1H) 2.58 (dd, 5H) 1.66 (s, 4H); MS m/z M+H 393, 395, M−H 391, 393.

### (ii) N-[(3R)-3,4-Dihydro-2H-chromen-3-yl]-2,2,2-trifluoroacetamide

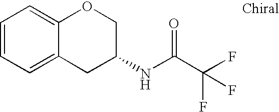

(3R)-3-[(2,2,2-Trifluoroacetyl)amino]-3,4-dihydro-2H-chromen-5-yl trifluoromethanesulfonate (2.0 g, 5.1 mmol) was dissolved in DMF (4 ml) and palladium (II) diacetate (6.2 mg, 0.25 mmol), triphenylphosphine (13.3 mg, 0.51 mmol), triethylamine (2.1 ml, 15 mmol) and formic acid (0.38 ml, 20 mmol) were added. The mixture was heated at 60° C. for 20 hours. Brine was added to the cooled mixture which was extracted with EtOAc (×3). The combined organic layers were washed with brine, dried (MgSO₄), filtered and the solvent was evaporated. The residue was purified by flash chromatography on silica eluting with heptane/EtOAc (gradient 5-30% EtOAc) to give a solid (1.1 g, 88%). ¹H NMR (400 MHz, CD₃OD) δ ppm 7.04-7.16 (m, 2H) 6.89 (t, 1H) 6.82 (d, 1H) 4.29-4.41 (m, 1H) 4.16-4.26 (m, 1H) 4.01 (dd, 1H) 3.13 (dd, 1H) 2.91 (dd, 1H); MS m/z M−H 244.

### (iii) N-[(3R)-6-Chloro-3,4-dihydro-2H-chromen-3-yl]-2,2,2-trifluoroacetamide

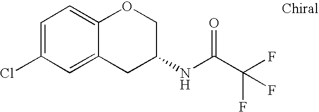

Sulfurylchloride (1M in dichloromethane, 0.30 ml, 0.30 mmol) was added dropwise to a solution of N-[(3R)-3,4-dihydro-2H-chromen-3-yl]-2,2,2-trifluoroacetamide (50 mg, 0.20 mmol) in acetic acid (0.30 ml). The mixture was stirred at ambient temperature for 1 hour. Sulfurylchloride (0.1 ml) was added and the mixture was stirred for an additional hour. Aqueous sodium hydrogen carbonate was added until pH 7-8 and the mixture was extracted with dichloromethane (×3). The combined organic layers were washed with brine, dried (MgSO₄) and the solvent was evaporated. The residue was purified by flash chromatography on silica eluting with heptane: EtOAc (gradient 5-20% EtOAc) to give a solid (50 mg, 93%), ¹H NMR (400 MHz, CD₃OD) δ ppm 7.04-7.16 (m, 2H) 6.81 (d, 1H) 4.28-4.41 (m, 1H) 4.16-4.26 (m, 1H) 4.04 (dd, 1H) 3.13 (dd, 1H) 2.89 (dd, 1H); MS m/z M−H 278, 280.

### (iv) (3R)-6-Chloro-3-[(trifluoroacetyl)amino]chromane-8-sulfonic acid,

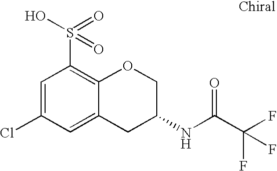

Chlorosulfonic acid (0.71 ml, 2.65 mmol) was added to N-[(3R)-6-chloro-3,4-dihydro-2H-chromen-3-yl]-2,2,2-trifluoroacetamide (0.74 g, 2.65 mmol) in chloroform (5 ml) at 0° C. The mixture was stirred at ambient temperature for 16 hours. The reaction mixture was poured onto ice. Water and dichloromethane was added and the layers were separated. The organic layer was extracted with water (×3). Sodium chloride was added to the aqueous layer until it was saturated. The aqueous layer was extracted with EtOAc (×3) and the combined organic layers were dried (MgSO₄) and the solvent was evaporated to give the crude title compound (0.98 g) that was used in the next step without further purification, MS m/z M−H 358, 360.

### (v) (3R)-6-chloro-3-[(trifluoroacetyl)amino]chromane-8-sulfonyl chloride

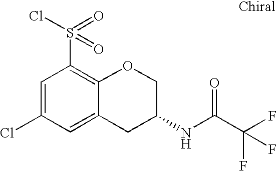

To 6-chloro-3-(2,2,2-trifluoro-acetylamino)-chroman-8-sulfonic acid (975 mg, 2.7 mmol) in dichloroethane (15 ml) and DMF (5 ml) was added thionyl chloride (4 ml). The reaction mixture was heated to reflux with a drying tube for 2 hours. The solvent was removed by evaporation. The crude was dissolved in dichloromethane and washed with saturated sodium hydrogen carbonate, which was reextracted with dichloromethane. The combined organic layers were dried (MgSO₄) and the solvent was removed by evaporation. The crude material was used directly in the next step. MS m/z M−H 376, 378.

### (vi) N-[(3R)-8-(Anilinosulfonyl)-6-chloro-3,4-dihydro-2H-chromen-3-yl]-2,2,2-trifluoroacetamide

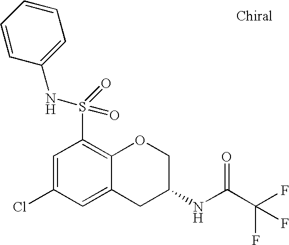

To 6-chloro-3-(2,2,2-trifluoro-acetylamino)-chroman-8-sulfonyl chloride (0.7 g, 1.8 mmol) in dichloromethane (10 ml) was added pyridine (165 μl, 2.05 mmol) followed by aniline (187 μl, 2.05 mmol). The reaction mixture was stirred at room temperature for 1 hour. The solvent was removed by evaporation and the crude mixture was purified by flash chromatography on silica (heptane:EtOAc, gradient; 80:20-50:50) to give the title compound (50.2 mg, 6.4%) as a solid. This solid was used directly in the next step. MS m/z M+H 435, 437, M−H 433, 435.

### (vii) (3R)-3-Amino-6-chloro-N-phenylchromane-8-sulfonamide

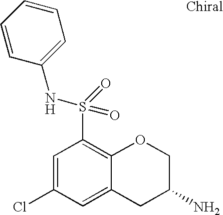

N-[(3R)-8-(Anilinosulfonyl)-6-chloro-3,4-dihydro-2H-chromen-3-yl]-2,2,2-trifluoroacetamide (50 mg, 0.115 mmol) was dissolved in chloroform (4 ml) and aqueous sodium hydroxide (2 M, 4 ml). The mixture was stirred at ambient temperature for 1.5 hours. Concentrated hydrochloric acid (800 μl) was added to reach acidic pH. Sodium hydrogen carbonate was added until basic pH was reached and the mixture was stirred for 16 hours. The layers were separated and the aqueous phase was extracted with dichloromethane (×2). The combined organic phases were dried (Na₂SO₄) and the solvents were evaporated. The crude (40 mg) was used in the next step without further purification. MS m/z M+H 339, 341, M−H 337, 339.

### Example 74

### (i) (3R)—N-(4-Chlorophenyl)-5-methoxy-3-(methylamino)chromane-8-sulfonamide

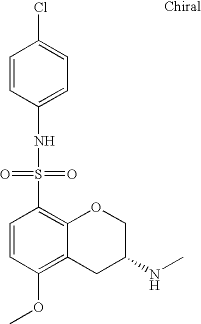

A solution of ethyl ((3R)-8-{[(4-chlorophenyl)amino]sulfonyl}-5-methoxy-3,4-dihydro-2H-chromen-3-yl)carbamate (85 mg, 0.193 mmol) in tetrahydrofuran (2 ml) was added to a suspension of lithium aluminumhydride (15 mg, 0.39 mmol) in tetrahydrofuran (1 ml) under and atmosphere of argon. The mixture was stirred at room temperature for 1 hr and then heated to reflux for 15 min. The reaction mixture was allowed to cool to RT and 2.5 ml sat. aqueous sodium sulfate solution was added. The resulting mixture was extracted with dichloromethane. The organic layer was dried over sodium sulfate, filtered and the solvent was removed under reduced pressure. The residue was purified by column chromatography on silica using gradient mixtures dichloromethane/methanol (0-20% methanol) as eluent. The title compound was isolated as a solid (24 mg, 32%).

¹H NMR (400 MHz, CDCl₃) δ ppm 7.63 (d, 1H) 7.12-7.18 (m, 2H) 7.04-7.10 (m, 2H) 6.41 (d, 1H) 4.34-4.41 (m, 1H) 4.04-4.12 (m, 1H) 3.83 (s, 3H) 3.02-3.10 (m, 1H) 2.83-2.92 (m, 1H) 2.54 (s, 3H) 2.47 (dd, 1H). MS m/z M+H 382.9, 384.9, M−H 381.1, 383.2.

### (ii) Ethyl ((3R)-8-{[(4-chlorophenyl)amino]sulfonyl}-5-methoxy-3,4-dihydro-2H-chromen-3-yl)carbamate

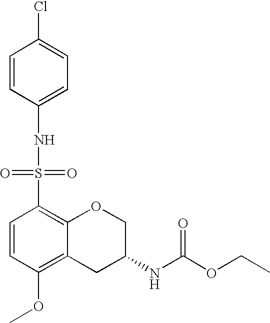

(3R)-3-amino-N-(4-chlorophenyl)-5-methoxychromane-8-sulfonamide (171 mg, 0.46 mmol) was dissolved in 5 ml dichloromethane. Ethylchloroformate (0.044 ml, 0.46 mmol) and pyridine (0.075 ml, 0.926 mmol) were added and the reaction mixture was stirred for 1 hour. Dichloromethane (20 ml) were added and the organic phase was washed with 1 M hydrochloric acid, water and sat. NaHCO₃ solution. The organic phase was dried (Na₂SO₄), filtered and the solvent was removed under reduced pressure. The crude product (90 mg, 44%) was used without further purification.

¹H NMR (400 MHz, CDCl₃) δ ppm 7.67 (d, 1H) 7.18 (d, 2H) 7.02-7.08 (m, 2H) 6.44 (d, 1H) 4.69-4.77 (m, 1H) 4.34-4.43 (m, 1H) 4.10-4.33 (m, 3H) 3.83 (s, 3H) 2.87 (dd, 1H) 2.63-2.72 (m, 1H) 1.21-1.31 (m, 3H). MS m/z M+H 440.7, 442.6, M+NH₄ 457.8, 459.7, M−H 438.8, 440.9.

### (iii) (3R)-3-amino-N-(4-chlorophenyl)-5-methoxychromane-8-sulfonamide

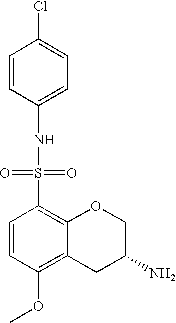

N-((3R)-8-{[(4-chlorophenyl)amino]sulfonyl}-5-methoxy-3,4-dihydro-2H-chromen-3-yl)-2,2,2-trifluoroacetamide (607 mg, 1.3 mmol) was stirred in 15 ml methanol. 2M sodium hydroxide (1.96 ml, 3.92 mmol) were added and the reaction mixture was stirred for 7 days. The pH was adjusted to ca. 6.5 by the addition of solid ammonium chloride. The methanol was removed under reduced pressure and the aqueous layer was made basic by addition of 1M sodium carbonate. The aqueous layer was extracted with dichloromethane twice, the combined extracts were dried (sodium sulfate), filtered and the solvent was removed under reduced pressure. The residue was purified using a SCX-2 column, washing with dichloromethane, methanol and eluting with 0.7M ammonia in methanol yielding the product (348 mg, 72%).

MS m/z M−H 367.

### (iv) N-((3R)-8-{[(4-chlorophenyl)amino]sulfonyl}-5-methoxy-3,4-dihydro-2H-chromen-3-yl)-2,2,2-trifluoroacetamide

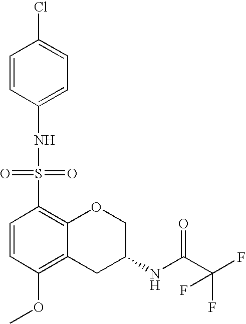

p-Chloroaniline (165 mg, 1.29 mmol) was added to a solution of (3R)-5-Methoxy-3-[(trifluoroacetyl)amino]chromane-8-sulfonyl chloride (460 mg, 1.23 mmol) in dichloromethane (5 ml) followed by the addition of pyridine (0.25 ml, 3.08 mmol) and the reaction mixture was stirred for 2 hours. Dichloromethane was added (20 ml) and the organic phase was washed with 1N hydrochloric acid, water, sat. aqueous sodium hydrogencarbonate solution and dried (sodium sulfate). The solvent was removed under reduced pressure and the product (613 mg, quant.) was used without further purification. MS m/z M+H 464.8 M−H 462.9, 464.9.

### Example 75

### (i) (3R)-5-Methoxy-3-(methylamino)-N-[4-(trifluoromethyl)phenyl]chromane-8-sulfonamide

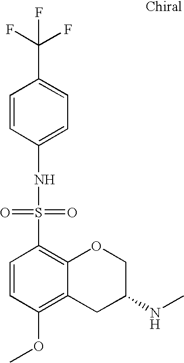

A solution of Ethyl [(3R)-5-methoxy-8-({[4-(trifluoromethyl)phenyl]amino}sulfonyl)-3,4-dihydro-2H-chromen-3-yl]carbamate (88 mg, 0.185 mmol) in anhydrous tetrahydrofuran (3 ml) was added to a suspension of lithium aluminumhydride (14 mg, 0.37 mmol) in anhydrous tetrahydrofuran (1 ml) under an atmosphere of argon. The mixture was stirred at room temperature for 10 min and then heated to reflux for 30 min. The mixture was cooled to room temperature and sat. sodium sulfate (0.5 ml) was carefully added. The mixture was diluted with dichloromethane (10 ml) and filtered, dried (sodium sulfate) and the solvent was removed. The product was purified using HPLC to yield 24 mg (31%) of product.

¹H NMR (400 MHz, CDCl₃) δ ppm 7.65 (d, 1H) 7.44 (d, 2H) 7.21 (d, 2H) 6.43 (d, 1H) δ 5.15-5.24 (m, 1H) 3.84 (s, 3H) 3.21 (dd, 1H) 2.75-2.89 (m, 3H) 2.50 (s, 3H). MS m/z M+H 416.6, M−H 414.6.

### (ii) Ethyl [(3R)-5-methoxy-8-({[4-(trifluoromethyl)phenyl]amino}sulfonyl)-3,4-dihydro-2H-chromen-3-yl]carbamate

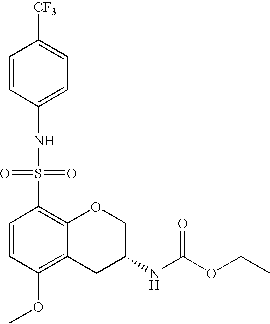

Ethyl chloroformate (0.029 ml, 0.31 mmol) and pyridine (0.049 ml, 0.61 mmol) were added to a solution of (3R)-3-amino-5-methoxy-N-[4-(trifluoromethyl)phenyl]chromane-8-sulfonamide (106 mg, 0.263 mmol) in dichloromethane (2 ml). After one hour more Ethyl chloroformate (0.049 ml) and pyridine (0.049 ml) were added and the reaction mixture was stirred for another hour. Dichloromethane was added (10 ml) and the organic phase was washed with 1 M hydrochloric acid and subsequently with sat. sodium hydrogencarbonate solution. The organic phase was dried (sodium sulfate), filtered and the solvent was removed under reduced pressure. The residue was purified by chromatography on silica using a gradient of heptane/ethyl acetate reaching from 0-100% of ethyl acetate to yield 93 mg (74%) of product.

¹H NMR (400 MHz, CDCl₃) δ ppm 7.75 (d, 1H) 7.47 (d, 2H) 7.21 (d, 2H) 6.47 (d, 1H) 4.70-4.76 (m, 1H) 4.33-4.41 (m, 1H) 4.09-4.25 (m, 3H) 3.83 (s, 3H) 2.86 (dd, 1H) 2.62-2.71 (m, 1H) 1.22-1.32 (m, 3H). MS m/z M+H 474.6, M+NH₄ 491.7, M−H 472.7.

### (iii) (3R)-3-amino-5-methoxy-N-[4-(trifluoromethyl)phenyl]chromane-8-sulfonamide

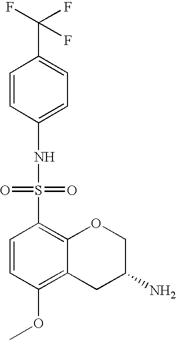

2,2,2-trifluoro-N-[(3R)-5-methoxy-8-({[4-(trifluoromethyl)phenyl]amino}sulfonyl)-3,4-dihydro-2H-chromen-3-yl]acetamide (152 mg, 0.31 mmol) and ammonia in methanol (7M, 0.436 ml), methanol (1 ml) and water (0.1 ml) were heated in a microwave oven at 140° C. for 20 min. The solvent was removed under reduced pressure and the product (136 mg, quant) was used without further purification.

MS m/z M+H 402.7, M−H 400.8.

### (iv) 2,2,2-trifluoro-N-[(3R)-2-methoxy-8-({[4-(trifluoromethyl)phenyl]amino}sulfonyl)-3,4-dihydro-2H-chromen-3-yl]acetamide

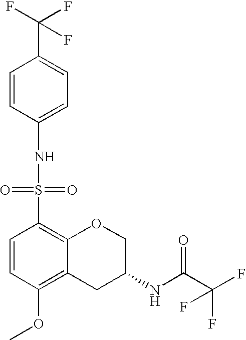

A solution of (3R)-5-Methoxy-3-[(trifluoroacetyl)amino]chromane-8-sulfonyl chloride (113 mg, 0.303 mmol) in dichloromethane (1 ml) and pyridine (0.061 ml, 0.76 mmol) was added to a solution of p-trifluoromethylaniline (59 mg, 0.364 mmol) in dichloromethane (0.5 ml). The reaction mixture was stirred for 8 hours and the solvent was removed under reduced pressure. The residue was purified by using a SCX-2 column washing with dichloromethane and eluting with 2% methanol in dichlormethane to yield the product (157 mg, quant.).

MS m/z M+H 498.6, M−H 496.7.

### Example 76

### (i) (3R)—N-(3,4-Dichlorophenyl)-5-methoxy-3-(methylamino)chromane-8-sulfonamide

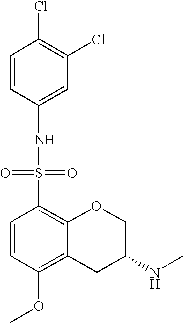

The title compound was prepared using the method described in Example 75 (i) (29 mg, 42%).

¹H NMR (400 MHz, CDCl₃) δ ppm 7.59 (d, 1H) 7.21-7.26 (m, 2H) 7.03 (dd, 1H) 6.42 (d, 1H) 5.15-5.25 (m, 1H) 3.84 (s, 3H) 3.23 (dd, 1H) 2.87 (d, 2H) 2.80 (dd, 1H) 2.53 (s, 3H). MS m/z M+H 416.6, 418.5, 420.5, M−H 414.6, 416.5, 418.6.

### (ii) ethyl ((3R)-8-{[(3,4-dichlorophenyl)amino]sulfonyl}-5-methoxy-3,4-dihydro-2H-chromen-3-yl)carbamate

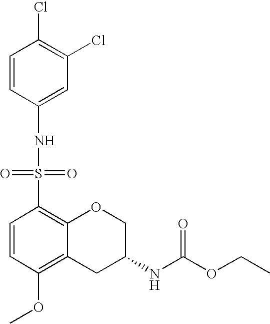

The title compound was prepared using the method described in Example 75 (ii) to give the product (68%).

MS m/z M+H 474.6, M−H 472.7, 474.6

### (iii) (3R)-3-amino-N-(3,4-dichlorophenyl)-5-methoxychromane-8-sulfonamide

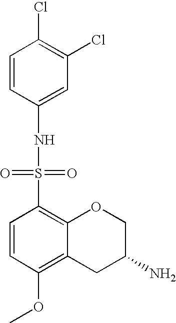

The title compound was prepared using the method described in Example 75 (iii) to give the product (quant.).

MS m/z M+H 402.7, 404.6, M−H 400.7, 402.7.

### (iv) N-((3R)-8-{[(3,4-dichlorophenyl)amino]sulfonyl}-5-methoxy-3,4-dihydro-2H-chromen-3-yl)-2,2,2-trifluoroacetamide

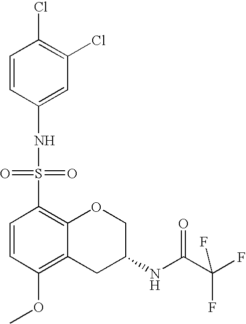

The title compound was prepared using the method described in Example 75 (iv) to yield the product (95%).

MS m/z M+H 498.6, 500.6 M−H 496.7, 498.6

### Example 77

### (i) (3R)-5-Methoxy-3-(methylamino)-N-[3-(trifluoromethyl)phenyl]chromane-8-sulfonamide

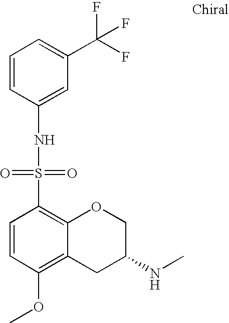

The title compound was prepared using the method described in Example 75 (i) to yield the product (40%).

¹H NMR (400 MHz, CDCl₃) δ ppm 7.61 (d, 1H) 7.22-7.43 (m, 4H) 6.40 (d, 1H) 5.13-5.23 (m, 1H) 3.83 (s, 3H) 3.20 (dd, 1H) 2.84-2.87 (m, 2H) 2.77 (dd, 1H) 2.51 (s, 3H). MS m/z M+H 416.7, M−H 414.7.

### (ii) ethyl [(3R)-5-methoxy-8-({[3-(trifluoromethyl)phenyl]amino}sulfonyl)-3,4-dihydro-2H-chromen-3-yl]carbamate

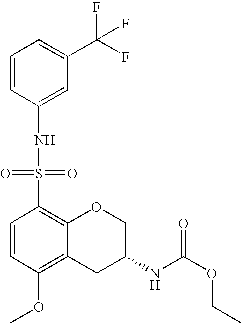

The title compound was prepared using the method described in Example 75 (ii) to give the product (79%).

MS m/z M+H 474.6, M−H 472.7.

### (iii) (3R)-3-amino-5-methoxy-N-[3-(trifluoromethyl)phenyl]chromane-8-sulfonamide

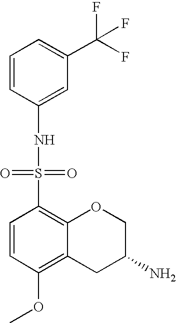

The title compound was prepared using the method described in Example 75 (iii) to yield the product (quant.).

MS m/z M+H 402.7, 420.5, M−H 400.8.

### (iv) 2,2,2-trifluoro-N-[(3R)-5-methoxy-8-({[3-(trifluoromethyl)phenyl]amino}sulfonyl)-3,4-dihydro-2H-chromen-3-yl]acetamide

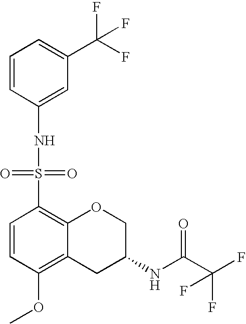

The title compound was prepared using the method described in Example 75 (iv) to yield the product (83%).

MS m/z M+H 498.7, M−H 496.8

### Example 78

### (i) (3R)-5-Methoxy-3-(methylamino)-N-quinolin-2-ylchromane-8-sulfonamide

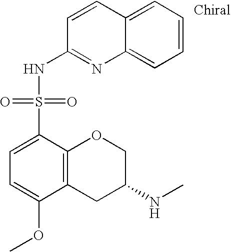

2,2,2-Trifluoro-N-{(3R)-5-methoxy-8-[(quinolin-2-ylamino)sulfonyl]-3,4-dihydro-2H-chromen-3-yl}-N-methylacetamide (90 mg) was dissolved in 3 ml tetrahydrofuran. 5N sodium hydroxide (2 ml) were added and the reaction mixture was stirred for 10 hours. 1N hydrochloric acid was added until neutral pH and sat. sodium hydrogencarbonate solution was added and the mixture was extracted with chloroform (twice). The combined organic phase was dried (sodium sulfate), filtered and the solvent was removed under reduced pressure. The residue was purified using a SCX column, washing with methanol and eluting with 0.7M ammonia in methanol. The product was then purified by chromatography on silica using a gradient of CHCl₃/MeOH/NH₃ reaching from 0-10% of methanol containing ammonia (3%) to yield the product (25 mg, 35%).

¹H NMR (400 MHz, CD₃OD) δ ppm 8.13 (d, 1H) 7.82 (d, 1H) 7.79 (m, 1H) 7.69 (m, 1H) 7.60 (d, 1H) 7.42 (m, 1H) 7.35 (m, 1H) 6.65 (d, 1H) 4.03 (m, 1H) 3.88 (s, 3H) 3.70 (m, 1H) 2.85-2.96 (m, 2H) 2.42 (m, 1H) 2.33 (m, 3H); MS m/z M+H 400.0, M−H 398.2.

### (ii) Ethyl [(3R)-5-methoxy-3,4-dihydro-2H-chromen-3-yl]carbamate

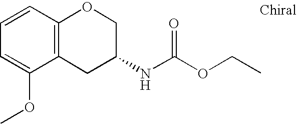

(3R)-5-methoxychroman-3-amine (14.7 g, 82 mmol) were dissolved in 200 ml dichloromethane and the solution was cooled to 0° C. Ethyl chloroformate (10 ml) and ethyldiisopropyl amine (21 ml, 128 mmol) were slowly added to the solution and stirring was continued for 10 min at 0° C. The reaction was allowed to warm up to room temperature and successively washed with sat. sodium hydrogencarbonate, 1N hydrochloric acid and again with sat sodium hydrogencarbonate solution. The organic phase was dried (sodium sulfate), filtered and the solvent was removed under reduced pressure. The product (19.7 g, 96%) was used without further purification.

MS m/z M+H 252.12

### (iii) (3R)-5-Methoxy-N-methylchroman-3-amine

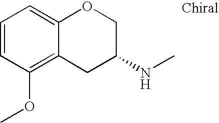

A solution of ethyl [(3R)-5-methoxy-3,4-dihydro-2H-chromen-3-yl]carbamate (10 g, 40 mmol) in tetrahydrofuran (50 ml), was slowly added to a suspension of lithium aluminum hydride (2.1 g, 55 mmol) in tetrahydrofuran (50 ml) under argon atmosphere. After completion of the addition and the evolution of hydrogen gas subsidized, the reaction mixture was heated to reflux for 24 hours. The reaction mixture was cooled to 5-10° C. with an ice bath and decomposed by cautious, dropwise addition of 2.1 ml of water, followed by 2.1 ml of 15% aq. sodium hydroxide and finally by 6.3 ml of water. The mixture was allowed to reach room temperature, stirred for an additional hour and filtered. The filtercake is washed with tetrahydrofuran and the combined filtrate was concentrated under reduced pressure to give the product, which was used without further purification. MS m/z M+H 194.02

### (iv) (3R)-5-Methoxy-3-[methyl(trifluoroacetyl)amino]chromate-8-sulfonyl chloride

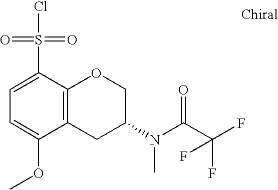

(3R)-5-Methoxy-N-methylchroman-3-amine (3.6 g, 18.5 mmol) was dissolved in chloroform (50 ml) and the mixture was cooled to 0° C. Trifluoroacetic anhydride (2.85 ml) and DIPEA (3.3 ml) was added slowly. The mixture was stirred at 0° C. for 5 min and then the mixture was allowed to reach room temperature and stirred for 2 hours. The mixture was washed with aqueous saturated sodium hydrogen carbonate, 1M hydrochloric acid and aqueous saturated sodium hydrogen carbonate. The organic layer was dried (sodium sulfate), filtered and the solvent was evaporated.

The residue (4.2 g) was dissolved in dichloromethane (10 ml) and the mixture was cooled to 0° C. Chlorosulfonic acid (1.9 ml, 28.2 mmol) in dichloromethane (10 ml) was added dropwise to the mixture. The mixture was stirred at 0° C. for 10 min and thionyl chloride (3.1 ml, 42.3 mmol) in dichloromethane (10 ml) was added dropwise. DMF (0.2 ml) was added and the mixture was stirred at RT for 20 hours. The mixture was poured onto ice and the phases were separated. The organic layer was washed with saturated aqueous sodium hydrogen carbonate (×2), dried (Na₂SO₄), filtered and the solvent was evaporated to give a solid (3.8 g). MS m/z M+H 388.

### (v) 2,2,2-Trifluoro-N-{(3R)-5-methoxy-8-[(quinolin-2-ylamino)sulfonyl]-3,4-dihydro-2H-chromen-3-yl}-N-methylacetamide

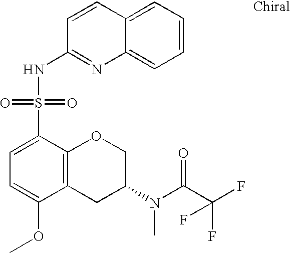

(3R)-5-Methoxy-3-[methyl(trifluoroacetyl)amino]chromane-8-sulfonyl chloride (790 mg, 2.0 mmol) and 2-aminoquinoline (340 mg, 2.4 mmol) were dissolved in chloroform (10 ml). DIPEA (0.9 ml) was added. The mixture was heated at 40° C. for 20 hours. Pyridine (0.6 ml) was added and the mixture was heated at 40° C. for 3 hours. The mixture was washed with 1M hydrochloric acid and saturated aqueous sodium hydrogen carbonate. The organic phase was dried (Na₂SO₄), filtered and the solvent was evaporated. The residue was purified by chromatography on silica using a gradient of CHCl₃/MeOH/NH₃ reaching from 0-10% of methanol containing ammonia (3%) to give the product (180 mg, 18%)

MS m/z M+H 496

### Example 79

### (i) (3R)—N-(3-Cyanophenyl)-5-methoxy-3-(methylamino)chromane-8-sulfonamide

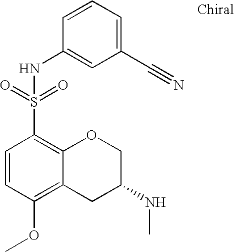

N-((3R)-8-{[(3-Cyanophenyl)amino]sulfonyl}-5-methoxy-3,4-dihydro-2H-chromen-3-yl)-2,2,2-trifluoro-N-methylacetamide (74 mg, 0.55 mmol) was dissolved in methanol (1.5 ml) and aqueous sodium hydroxide (2M, 0.75 ml) was added. The mixture was stirred at ambient temperature for 20 hours. The mixture was concentrated under reduced pressure, diluted with water and extracted with EtOAc (×3) and dichloromethane. The combined organic layers were dried (Na₂SO₄), filtered and evaporated. The residue was purified by chromatography on silica using a gradient of CHCl₃/MeOH/NH₃ reaching from 0-10% of methanol containing ammonia (3%) to give a solid (26 mg, 46%). ¹H NMR (400 MHz, CDCl₃) δ ppm 7.63 (1H, d) 7.46-7.49 (1H, m) 7.41-7.45 (1H, m) 7.31-7.36 (2H, m) 6.44 (1H, d) 4.36 (1H, d) 4.14-4.21 (1H, m) 3.84 (3H, s) 3.09-3.16 (1H, m) 2.89 (1H, dd) 2.59 (3H, s) 2.53 (1H, dd); MS m/z M+H 374.0, M−H 372.1

### (ii) N-((3R)-8-{[(3-Cyanophenyl)amino]sulfonyl}-5-methoxy-3,4-dihydro-2H-chromen-3-yl)-2,2,2-trifluoro-N-methylacetamide

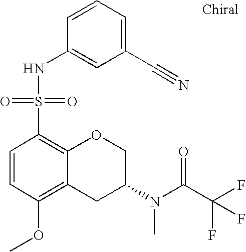

(3R)-5-Methoxy-3-[methyl(trifluoroacetyl)amino]chromane-8-sulfonyl chloride (194 mg, 0.5 mmol), 3-aminobenzonitrile (118 mg, 1.0 mmol) and pyridine (200 μl, 2.5 mmol) were dissolved in dichloromethane (3 ml). The mixture was stirred at ambient temperature for 20 hours. EtOAc was added and the mixture was washed with hydrochloric acid (1M), aqueous sodium hydroxide (1M) and water. The organic phase was dried (Na₂SO₄), filtered and the solvent was evaporated. The residue was purified by chromatography on silica using a gradient of heptane/ethyl acetate reaching from 0-100% of ethyl acetate to give a solid (51 mg, 21%).

MS m/z M−H 468.1

### Example 80

### (i) (3R)—N-(4-Cyanophenyl)-5-methoxy-3-(methylamino)chromane-8-sulfonamide

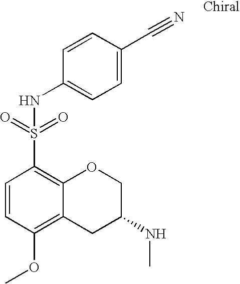

The title compound was prepared using the method described in Example 79 (i) to give a solid (29 mg, 45%), ¹H NMR (400 MHz, CDCl₃) δ ppm 7.72 (1H, d) 7.49 (2H, d) 7.20 (2H, d) 6.46 (1H, d) 4.32-4.38 (1H, m) 4.01-4.07 (1H, m) 3.84 (3H, s) 3.01-3.09 (1H, m) 2.87 (1H, dd) 2.54 (3H, s) 2.46 (1H, dd); MS m/z M+H 374.0, M−H 372.1

### (ii) N-((3R)-8-{[(4-Cyanophenyl)amino]sulfonyl}-5-methoxy-3,4-dihydro-2H-chromen-3-yl)-2,2,2-trifluoro-N-methylacetamide

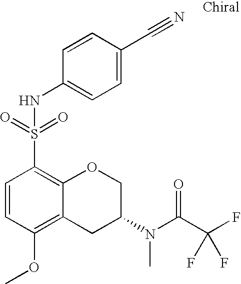

The title compound was prepared using the method described in Example 79 (ii) to give a solid (69 mg, 29%). MS m/z M−H 468.1

### Example 81

### (3R)—N-(4-Chlorophenyl)-3-(dimethylamino)-5-methoxychromane-8-sulfonamide

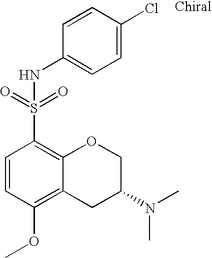

(3R)—N-(4-Chlorophenyl)-5-methoxy-3-(methylamino)chromane-8-sulfonamide (38 mg, 0.10 mmol) was dissolved in methanol (2 ml) and formaldehyde (33% aqueous solution, 50 μl, 0.50 mmol) and acetic acid (2 μl) were added. The mixture was stirred at ambient temperature for 1 hour. Sodium cyanoborohydride (32 mg, 0.50 mmol) was added and the mixture was stirred at ambient temperature for 20 hours. Hydrochloric acid (2M, 0.5 ml) was added and the mixture was concentrated under reduced pressure. Aqueous sodium hydroxide (1M) was added until pH 8-9. The mixture was extracted with EtOAc (×3) and the combined organic phases were dried (Na₂SO₄), filtered and the solvent was evaporated. The residue was purified by chromatography on silica using a gradient of CHCl₃/MeOH/NH₃ reaching from 0-10% of methanol containing ammonia (3%) to give a solid (39 mg, 99%).

¹H NMR (400 MHz, CDCl₃) δ ppm 7.62 (1H, d) 7.16 (2H, d) 7.07 (2H, d) 6.43 (1H, d) 4.56-4.63 (1H, m) 3.85 (3H, s) 2.87-2.97 (1H, m) 2.60 (4H, br. s.) 2.46 (4H, br. s.); MS m/z M+H 395.1, M−H 397.0

### Example 82

### (3R)—N-(3-Cyanophenyl)-3-(dimethylamino)-5-methoxychromane-8-sulfonamide

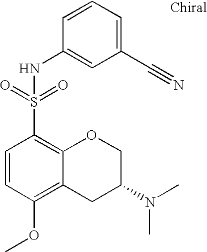

(3R)—N-(3-Cyanophenyl)-5-methoxy-3-(methylamino)chromane-8-sulfonamide (24 mg, 0.060 mmol) was dissolved in methanol (1 ml). Formaldehyde (33% aqueous solution, 55 μl, 0.60 mmol) and acetic acid (2 μl) were added and the mixture was stirred at ambient temperature for 1 hour. Sodium cyanoborohydride (19 mg, 0.30 mmol) was added and the mixture was stirred at ambient temperature for 20 hours. Hydrochloric acid (1M) was added to quench the reaction and the mixture was concentrated under reduced pressure. Aqueous sodium hydroxide (1M) was added until pH 8-9. The mixture was extracted with chloroform (×3) and the combined organic phases were dried (Na₂SO₄), filtered and the solvent was evaporated. The residue was purified by chromatography on silica using a gradient of CHCl₃/MeOH/NH₃ reaching from 0-10% of methanol containing ammonia (3%) to give a solid (16 mg, 70%). ¹H NMR (400 MHz, CDCl₃) δ ppm 7.64 (1H, d) 7.31-7.43 (4H, m) 6.45 (1H, d) 4.54 (1H, d) 4.11 (1H, br. s.) 3.85 (3H, s) 2.82-2.91 (1H, m) 2.60-2.78 (2H, m) 2.43 (6H, br. s.); MS m/z M+H 388.0, M−H 386.2.

### Example 83

### (3R)—N-(4Cyanophenyl)-3-(dimethylamino)-5-methoxychromane-8-sulfonamide

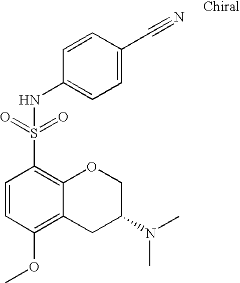

The title compound was prepared using the method described in Example 82 to give a solid (24 mg, 99%).

¹H NMR (400 MHz, CDCl₃) δ ppm 7.22 (1H, d) 7.49 (2H, m) 7.21 (2H, m) 6.49 (1H, d) 4.58 (1H, br. s.) 3.99 (1H, br. s.) 3.86 (3H, s) 2.83-2.95 (1H, m) 2.52-2.64 (2H, m) 2.48 (6H, br. s.);

MS m/z M+H 388.0, M−H 386.2

### Pharmacology

Method for [125I]SB258585 binding to rat striatal 5HT6 receptors

### Materials

[¹²⁵I]SB258585 (1) with specific radioactivity 2000 Ci/mmol was purchased from Amersham Biosciences Europe GmbH, Freiburg, Germany. Other chemicals were purchased from commercial sources and were of analytical grade.

### Preparation of Membranes:

Striatal tissue from adult rats (Sprague-Dawley, 320-370 g, B & K Sweden) were dissected out, weighed and homogenized in buffer containing 50 mM Tris-HCl, 4 mM MgCl2, 1 mM EDTA, 10 μM pargyline and protease inhibitor (Complete, Roche Diagnostics) pH 7.4 using an Ultra-Turrax T8 (IKA Labortechnik, Germany). The tissue homogenate was centrifuged at 48 000×g for 10 min and the pellet was resuspended and recentrifuged as above. The final membranes were diluted in buffer to a concentration of 60 mg original wet weight (w.w.) per ml and stored in aliquots at −70° C.

### Radioligand Binding Assays:

Saturation binding studies were carried out in duplicate with 1-3 mg w.w. per tube in 0.5 ml buffer (50 mM Tris, 4 mM MgCl2, 100 mM NaCl, 1 mM EDTA, 5 mM ascorbate and 10 μM pargyline at pH 7.4), 0.2 nM [¹²⁵I]SB258585 and unlabelled SB258585 to give a final concentration range of 0.23-20 nM (12 conc.). Non-specific binding was determined in the presence of 10 μM methiothepin. In the competition experiments 0.8-2 mg w.w. per tube and a radioligand concentration of 0.5-1 nM were used with 7 concentrations of the competing drug pre-dissolved in DMSO and diluted in buffer. The assays were incubated for 1-3 hours at room temperature, and terminated by rapid filtration through Whatman GF/B filters pretreated with 0.3% polyethyleneimine using a Brandel cell harvester. The radioactivity was determined in a Packard Tri-Carb 2900TR liquid scintillation counter. Data were analyzed by non-linear regression analyses using PRISM 4.00 (GraphPad Software Inc., San Diego, Calif.).

More information about the assay can be found in Hirst, W. D., Minton, J. A. L., Bromidge, S. M., Moss, S. F., Latter, A., Riley, G., Routledge, C., Middlemiss, D. N. & Price, G. W. (2000). Characterization of [¹²⁵I]-SB-258585 binding to human recombinant and native 5HT6 receptors in rat, pig and human brain tissue is described in Br. J. Pharmacol., 130, 1597-1605.

### Results

Typical IC₅₀ values as measured in the assays described above are 1 μM or less. In one aspect of the invention the IC₅₀ is below 500 nM. In another aspect of the invention the IC₅₀ is below 50 nM. In a further aspect of the invention the IC₅₀ is below 10 nM.

TABLE 1
Specimen results from assay.

| Example no | Kᵢ (nM) |
|---|---|
| 1 | 1.7 ± 0.6 |
| 21 | 4.2 ± 1.4 |
| 25 | 12 ± 4.2 |
| 18 | 17 ± 5.0 |
| 69 | 27 ± 16 |
| 60 | 2.3 ± 1.3 |
| 71 | 30 ± 7 |
| 72 | 290 ± 50 |

1. A compound having the formula I 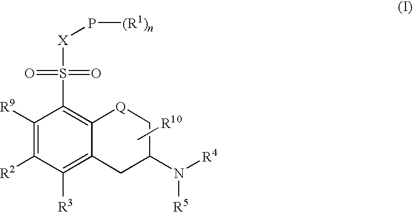
wherein:
P is C₆₋₁₀arylC₀₋₆alkyl, C₅₋₁₁heteroarylC₀₋₆alkyl, C₃₋₇cycloalkylC₀₋₆alkyl, C₃₋₇heterocycloalkylC₀₋₆alkyl or C₁₋₁₀alkyl;
R¹ is hydrogen, hydroxy, halogen, C₁₋₁₀alkyl, C₂₋₁₀alkenyl, C₂₋₁₀alkynyl, C₁₋₁₀alkoxy, N(R¹¹)₂, C₆₋₁₀arylC₀₋₆alkyl, C₅₋₆heteroarylC₀₋₆alkyl, C₁₋₆haloalkyl, C₁₋₆haloalkylO, R⁷OC₀₋₆alkyl, cyano, SR⁷, R⁷SO₂C₀₋₆alkyl, SOR⁷, R⁷CON(R⁸)C₀₋₆alkyl, NR⁸SO₂R⁷, COR⁷, COOR⁷, OSO₂R⁷, (R⁸)₂NCOC₀₋₆alkyl, SO₂N(R⁸)₂, N(R⁸)CON(R⁸)₂, NO₂ or oxo;
n is 0, 1, 2, 3 or 4;
X is a single bond, O, C₁₋₃alkyl or NR⁶, or X is N in a C₅₋₁₂heteroaryl;
Q is CH or O;
R² is hydrogen, hydroxy, halogen, C₀₋₁₀alkyl, C₂₋₁₀alkenyl, C₂₋₁₀alkynyl, C₁₋₁₀alkoxy, N(R¹¹)₂, C₆₋₁₀arylC₀₋₆alkyl, C₅₋₆heteroarylC₀₋₆alkyl, C₁₋₆haloalkyl, C₁₋₆haloalkylO, R⁷OC₀₋₆alkyl, cyano, SR⁷, SO₂R⁸, SOR⁷, N(R⁸)COR⁷, N(R⁸)SO₂R⁷, COR⁷, COOR⁷, OSO₂R⁷, CON(R⁸)₂ or SO₂N(R⁸)₂;
R³ is hydrogen, hydroxy, halogen, C₁₋₁₀alkyl, C₂₋₁₀alkenyl, C₂₋₁₀alkynyl, C₁₋₁₀alkoxy, N(R¹¹)₂, C₆₋₁₀arylC₀₋₆alkyl, C₅₋₆heteroarylC₀₋₆alkyl, C₁₋₆haloalkyl, C₁₋₆haloalkylO, R⁷OC₀₋₆alkyl, cyano, SR⁷, SO₂R⁷, SOR⁷, N(R⁸)COR⁷, N(R⁸)SO₂R⁷, COR⁷, COOR⁷, OSO₂R⁷, CON(R⁸)₂ or SO₂N(R⁸)₂;
R⁴ and R⁵ are selected independently from hydrogen, C₁₋₅alkyl, C₁₋₅haloalkyl, C₂₋₅alkenyl, C₂₋₅alkynyl, C₃₋₆cycloalkyl, C₅₋₆arylC₁₋₂alkyl and C₅₋₆heteroarylC₁₋₂alkyl, wherein R⁴ and R⁵ are optionally substituted by one or more groups selected independently from halogen, hydroxyl, cyano and C₁₋₅alkoxy, or
R⁴ and R⁵ form together a C₃₋₇heterocycloalkyl optionally substituted by one or more groups independently selected from hydrogen, halogen, C₁₋₆alkyl, C₁₋₆haloalkyl, COR¹², OR¹², SO₂R¹², SO₂N(R¹¹)₂, C₅₋₆aryl, C₅₋₆heteroaryl, cyano, and oxo substituted on β or γ position;
R⁶ is hydrogen, C₁₋₆alkyl, C₃₋₆cycloakyl, R⁷OC₁₋₆alkyl, C₁₋₆haloalkyl, C₁₋₆cyanoalkyl, (R¹¹)₂NCOC₀₋₆alkyl or R¹²SO₂C₁₋₆alkyl;
R⁷ is C₁₋₁₀alkyl, C₆₋₁₀arylC₀₋₆alkyl, C₅₋₆heteroarylC₀₋₆alkyl, C₃₋₇cycloalkylC₀₋₆alkyl or C₁₋₆haloalkyl;
R⁸ is hydrogen, C₁₋₁₀alkyl, C₁₋₆haloalkyl, C₃₋₇cycloalkylC₀₋₆alkyl, C₆₋₁₀arylC₀₋₆alkyl or C₅₋₆heteroarylC₀₋₆alkyl, or
R⁷ and R⁸ form together a C₅₋₆heteroaryl or C₃₋₇heterocycloalkyl,
wherein each R¹, R⁷ and R⁸ aryl and each R¹, R⁷ and R⁸ heteroaryl is optionally and independently substituted by one or more groups selected independently from hydrogen, halogen, hydroxy, C₁₋₆haloalkyl, cyano, OR¹², C₁₋₆alkyl, oxo, SR¹¹, CON(R¹¹)₂, N(R¹¹)COR¹², SO₂R¹², SOR¹², N(R¹¹)₂, and COR¹²;
R⁹ is hydrogen, halogen, hydroxy, C₁₋₆alkoxy, C₁₋₆haloalkoxy, C₁₋₆haloalkyl, C₁₋₆alkyl or COR¹²;
R¹⁰ is hydrogen, C₁₋₆alkyl, C₁₋₆alkoxy or C₁₋₆haloalkyl;
R¹¹ is hydrogen, C₁₋₆alkyl or C₁₋₆haloalkyl; and
R¹² is C₁₋₆alkyl or C₁₋₆haloalkyl, or
R¹¹ and R¹² form together a C₃₋₇cycloalkyl or C₃₋₇heterocycloalkyl, wherein R¹¹ and R¹² are optionally independently substituted by one or more groups independently selected from hydrogen, halogen, hydroxy, cyano, C₁₋₃alkyl, C₁₋₃alkoxy, and C₁₋₃haloalkyl,
or salts, solvates, or solvated salts thereof.

2. The compound according to claim 1, wherein:
P is C₆₋₁₀arylC₀₋₃alkyl, C₅₋₁₁heteroarylC₀₋₃alkyl or C₃₋₇cycloalkylC₀₋₃alkyl;
R¹ is hydrogen, halogen, C₁₋₁₀alkoxy, C₁₋₆haloalkyl or R⁷OC₀₋₆alkyl;
n is 0, 1, 2 or 3;
X is a single bond, O or NR⁶, or X is N in a C₅₋₁₂heteroaryl;
Q is CH or O;
R² is hydrogen or halogen;
R³ is hydrogen, C₁₋₁₀alkyl or C₁₋₁₀alkoxy;
R⁴ and R⁵ are independently selected from hydrogen, C₁₋₅alkyl, and C₁₋₅haloalkyl, or R⁴ and R⁵ form together a C₃₋₇heterocycloalkyl optionally substituted by one or more groups independently selected from hydrogen, C₅₋₆aryl, and C₅₋₆heteroaryl;
R⁵ is hydrogen or C₁₋₆cyanoalkyl;
R⁷ is C₁₋₁₀alkyl or C₃₋₇cycloalkylC₀₋₄alkyl;
R⁹ is hydrogen; and
R¹⁰ is hydrogen;
or salts, solvates, or solvated salts thereof.

3. The compound according to claim 1, wherein P is phenyl, naphtyl, pyridinyl, pyrimidyl, quinoline, iso-quinoline, cyclohexyl, 1,2-methylenedioxybenzene, or tetraline.

4. The compound according to claim 1, wherein R¹ is hydrogen, chloro, fluoro, bromo, methoxy, ethoxy, propoxy, fluoromethyl, difluoromethyl, trifluoromethyl, fluoromethoxy, difluoromethoxy, trifluoromethoxy, or cyano.

5. The compound according to claim 1, wherein R³ is methyl, ethyl, methoxy, ethoxy, propoxy, hydrogen, halogen, fluoromethyl, difluoromethyl, trifluoromethyl, fluoromethoxy, difluoromethoxy, or trifluoromethoxy.

6. The compound according to claim 1, wherein X is NR⁶ or O, or X is N in an indol, indoline, tetrahydroquinoline, tetrahydroisoquinoline, benzoxazepine, isoindoline, pyrrole, oxindole, or benzazepine.

7. The compound according to claim 1, wherein R⁴ and R⁵ are selected independently from hydrogen, methyl, ethyl, i-propyl, n-propyl, and fluoroethyl, or R⁴ and R⁵ form together pyrrolidine or morpholine.

8. The compound according to claim 1, wherein R⁶ is hydrogen, methyl, cyanomethyl, or fluoroethyl.

9. A compound according to claim 1, wherein said compound is selected from
(6S)—N-(5-Chloro-2-methoxyphenyl)-6-(dimethylamino)-4-methoxy-5,6,7,8-tetrahydronaphthalene-1-sulfonamide;
(6S)-6-(Dimethylamino)-4-methoxy-N-phenyl-5,6,7,8-tetrahydronaphthalene-1-sulfonamide;
(6S)—N-(3,5-Dichloro-2-methoxyphenyl)-6-(dimethylamino)-4-methoxy-5,6,7,8-tetrahydronaphthalene-1-sulfonamide;
(6S)-6-(Dimethylamino)-N-(3-fluorophenyl)-4-methoxy-5,6,7,8-tetrahydronaphthalene-1-sulfonamide;
(6R)-6-(Dimethylamino)-4-methoxy-N-phenyl-5,6,7,8-tetrahydronaphthalene-1-sulfonamide;
(6R)-6-(Dimethylamino)-N-(3-fluorophenyl)-4-methoxy-5,6,7,8-tetrahydronaphthalene-1-sulfonamide;
(6R)—N-(5-Chloro-2-methoxyphenyl)-6-(dimethylamino)-4-methoxy-5,6,7,8-tetrahydronaphthalene-1-sulfonamide;
(6S)—N-(3,5-Dichlorophenyl)-6-(dimethylamino)-4-methoxy-5,6,7,8-tetrahydronaphthalene-1-sulfonamide;
(6S)—N-(3-Chloro-4-fluorophenyl)-6-(dimethylamino)-4-methoxy-5,6,7,8-tetrahydronaphthalene-1-sulfonamide;
(6S)-6-(Dimethylamino)-N-(6-fluoropyridin-3-yl)-4-methoxy-5,6,7,8-tetrahydronaphthalene-1-sulfonamide;
(6S)-6-(Dimethylamino)-4-methoxy-N-[(2S)-8-methoxy-1,2,3,4-tetrahydronaphthalen-2-yl]-5,6,7,8-tetrahydronaphthalene-1-sulfonamide;
(6S)—N-(3,5-Dichlorophenyl)-6-[isopropyl(methyl)amino]-4-methoxy-5,6,7,8-tetrahydronaphthalene-1-sulfonamide;
(6S)—N-(5-Chloro-2-methoxyphenyl)-4-methoxy-6-pyrrolidin-1-yl-5,6,7,8-tetrahydronaphthalene-1-sulfonamide;
(6S)—N-(3,5-Dichlorophenyl)-4-methoxy-6-pyrrolidin-1-yl-5,6,7,8-tetrahydronaphthalene-1-sulfonamide;
(6S)—N-(3-Chloro-4-fluorophenyl)-4-methoxy-6-morpholin-4-yl-5,6,7,8-tetrahydronaphthalene-1-sulfonamide;
(6S)-4-Methoxy-6-(methylamino)-N-phenyl-5,6,7,8-tetrahydronaphthalene-1-sulfonamide;
(6S)-6-(Dimethylamino)-4-methoxy-N-pyrimidin-2-yl-5,6,7,8-tetrahydronaphthalene-1-sulfonamide;
(6S)-6-(Dimethylamino)-4-methoxy-N-pyridin-2-yl-5,6,7,8-tetrahydronaphthalene-1-sulfonamide;
(6S)-6-(Dimethylamino)-4-methoxy-N-quinolin-2-yl-5,6,7,8-tetrahydronaphthalene-1-sulfonamide;
4-Methoxy-6-pyrrolidin-1-yl-5,6,7,8-tetrahydro-naphthalene-1-sulfonic acid 3,4-dichloro-phenyl ester;
[5-(3,4-Dihydro-1H-isoquinoline-2-sulfonyl)-8-methoxy-1,2,3,4-tetrahydro-naphthalen-2-yl]-dimethyl-amine;
(6S)—N-cyclohexyl-6-(dimethylamino)-4-methoxy-5,6,7,8-tetrahydronaphthalene-1-sulfonamide;
(6S)—N-(3-Chloro-4-fluorophenyl)-4-methoxy-6-pyrrolidin-1-yl-5,6,7,8-tetrahydronaphthalene-1-sulfonamide;
(6S)—N-(5-chloro-2-methoxyphenyl)-N-(cyanomethyl)-4-methoxy-6-pyrrolidin-1-yl-5,6,7,8-tetrahydronaphthalene-1-sulfonamide;
(6S)—N-(4-chlorophenyl)-4-methoxy-6-(methylamino)-5,6,7,8-tetrahydronaphthalene-1-sulfonamide;
(6S)-4-methoxy-6-pyrrolidin-1-yl-N-[3-(trifluoromethyl)phenyl]-5,6,7,8-tetrahydronaphthalene-1-sulfonamide;
(6S)-4-methoxy-N-phenyl-6-pyrrolidin-1-yl-5,6,7,8-tetrahydronaphthalene-1-sulfonamide;
(6S)-6-[(2-fluoroethyl)amino]-4-methoxy-N-phenyl-5,6,7,8-tetrahydronaphthalene-1-sulfonamide;
(2S)-5-(2,3-dihydro-1H-indol-1-ylsulfonyl)-8-methoxy-N,N-dimethyl-1,2,3,4-tetrahydronaphthalen-2-amine;
(6S)—N-(5-chloro-2-methoxyphenyl)-4-methoxy-6-(methylamino)-5,6,7,8-tetrahydronaphthalene-1-sulfonamide;
(6S)—N-(4-chlorophenyl)-6-(dimethylamino)-4-methoxy-5,6,7,8-tetrahydronaphthalene-1-sulfonamide;
2-{[(6S)-4-methoxy-6-pyrrolidin-1-yl-5,6,7,8-tetrahydronaphthalen-1-yl]sulfonyl}-1,2,3,4-tetrahydroisoquinoline-7-carbonitrile;
(6S)—N-(4-chlorophenyl)-4-methoxy-6-pyrrolidin-1-yl-5,6,7,8-tetrahydronaphthalene-1-sulfonamide;
(6S)—N-(3,4-dichlorophenyl)-4-methoxy-6-pyrrolidin-1-yl-5,6,7,8-tetrahydronaphthalene-1-sulfonamide;
(6S)—N-(3,4-difluorophenyl)-4-methoxy-6-pyrrolidin-1-yl-5,6,7,8-tetrahydronaphthalene-1-sulfonamide;
(6S)—N-(5-chloropyridin-2-yl)-6-(dimethylamino)-4-methoxy-5,6,7,8-tetrahydronaphthalene-1-sulfonamide;
(6S)-6-(dimethylamino)-4-methoxy-N-pyridin-3-yl-5,6,7,8-tetrahydronaphthalene-1-sulfonamide;
(6S)—N-1,3-benzodioxol-5-yl-4-methoxy-6-pyrrolidin-1-yl-5,6,7,8-tetrahydronaphthalene-1-sulfonamide;
(6S)—N-(5-chloro-2-methoxyphenyl)-6-[(2-fluoroethyl)amino]-4-methoxy-5,6,7,8-tetrahydronaphthalene-1-sulfonamide;
(6S)—N-(5-chloro-2-methoxyphenyl)-6-[(2-fluoroethyl)(methyl)amino]-4-methoxy-5,6,7,8-tetrahydronaphthalene-1-sulfonamide;
(6S)-4-methoxy-6-(methylamino)-N-[4-(trifluoromethyl)phenyl]-5,6,7,8-tetrahydronaphthalene-1-sulfonamide;
(6S)—N-(4-chlorophenyl)-4-methoxy-N-methyl-6-(methylamino)-5,6,7,8-tetrahydronaphthalene-1-sulfonamide;
(2S)-5-(1H-indol-1-ylsulfonyl)-8-methoxy-N-methyl-1,2,3,4-tetrahydronaphthalen-2-amine;
(2S)-5-[(5-chloro-1H-indol-1-yl)sulfonyl]-8-methoxy-N-methyl-1,2,3,4-tetrahydronaphthalen-2-amine;
(2S)-8-methoxy-N-methyl-5-{[6-(trifluoromethyl)-1H-indol-1-yl]sulfonyl}-1,2,3,4-tetrahydronaphthalen-2-amine;
1-{[(6S)-4-methoxy-6-(methylamino)-5,6,7,8-tetrahydronaphthalen-1-yl]sulfonyl}-1H-indole-6-carbonitrile;
(2S)-5-[(7-fluoro-1H-indol-1-yl)sulfonyl]-8-methoxy-N-methyl-1,2,3,4-tetrahydronaphthalen-2-amine;
(2S)-5-[(4-fluoro-1H-indol-1-yl)sulfonyl]-8-methoxy-N-methyl-1,2,3,4-tetrahydronaphthalen-2-amine;
(2S)-8-methoxy-5-[(4-methoxy-1H-indol-1-yl)sulfonyl]-N-methyl-1,2,3,4-tetrahydronaphthalen-2-amine;
(2S)-5-(5H-[1,3]dioxolo[4,5-f]indol-5-ylsulfonyl)-8-methoxy-N-methyl-1,2,3,4-tetrahydronaphthalen-2-amine;
(2S)-5-[(7-chloro-1H-indol-1-yl)sulfonyl]-8-methoxy-N-methyl-1,2,3,4-tetrahydronaphthalen-2-amine;
(2S)-8-methoxy-N-methyl-5-(1H-pyrrolo[2,3-b]pyridin-1-ylsulfonyl)-1,2,3,4-tetrahydronaphthalen-2-amine;
(6S)-6-(dimethylamino)-4-methoxy-N-quinolin-3-yl-5,6,7,8-tetrahydronaphthalene-1-sulfonamide;
(6S)-6-(dimethylamino)-N-isoquinolin-3-yl-4-methoxy-5,6,7,8-tetrahydronaphthalene-1-sulfonamide;
(6S)—N-1,3-benzothiazol-6-yl-6-(dimethylamino)-4-methoxy-5,6,7,8-tetrahydronaphthalene-1-sulfonamide;
(2S)-5-[(3-chloro-1H-pyrrolo[2,3-b]pyridin-1-yl)sulfonyl]-8-methoxy-N,N-dimethyl-1,2,3,4-tetrahydronaphthalen-2-amine;
(2S)-5-(1H-benzimidazol-1-ylsulfonyl)-8-methoxy-N,N-dimethyl-1,2,3,4-tetrahydronaphthalen-2-amine;
(6S)—N-(4-cyanophenyl)-4-methoxy-6-(methylamino)-5,6,7,8-tetrahydronaphthalene-1-sulfonamide; and
(6S)-6-(methylamino)-N-[4-(trifluoromethyl)phenyl]-5,6,7,8-tetrahydronaphthalene-1-sulfonamide;
or salts, solvates, or solvated salts thereof.

10. A compound according to claim 1, wherein said compound is selected from
(3R)—N-(5-Chloro-2-methoxyphenyl)-3-(dimethylamino)-5-methoxychromane-8-sulfonamide;
(3R)—N-(5-Chloro-2-methoxyphenyl)-3-(diethylamino)-5-methoxychromane-8-sulfonamide;
(3R)—N-(5-Chloro-2-methoxyphenyl)-3-(dipropylamino)-5-methoxychromane-8-sulfonamide;
(3R)—N-(5-Chloro-2-methoxyphenyl)-5-methoxy-3-pyrrolidin-1-ylchromane-8-sulfonamide;
(3R)—N-(3-Chloro-4-fluorophenyl)-3-(dimethylamino)-5-methoxychromane-8-sulfonamide;
(3R)—N-(3-Chloro-4-fluorophenyl)-3-(isopropylamino)-5-methoxychromane-8-sulfonamide;
(3R)—N-(3-Chloro-4-fluorophenyl)-3-[isopropyl(methyl)amino]-5-methoxychromane-8-sulfonamide;
(3R)—N-(3-Chloro-4-fluorophenyl)-5-methoxy-3-pyrrolidin-1-ylchromane-8-sulfonamide;
(3R)—N-(3,5-Dichlorophenyl)-3-(dimethylamino)-5-methoxychromane-8-sulfonamide;
(3R)—N-(3,5-Dichlorophenyl)-5-methoxy-3-pyrrolidin-1-ylchromane-8-sulfonamide;
(3R)-3-(Dimethylamino)-5-methoxy-N-phenylchromane-8-sulfonamide;
(3R)-5-Methoxy-3-(methylamino)-N-phenylchromane-8-sulfonamide;
(3R)—N-(3-Chloro-4-fluorophenyl)-3-(dimethylamino)-5-ethylchromane-8-sulfonamide;
(3R)-6-Chloro-N-phenyl-3-pyrrolidin-1-ylchromane-8-sulfonamide;
(3R)—N-(4-Chlorophenyl)-5-methoxy-3-(methylamino)chromane-8-sulfonamide;
(3R)-5-Methoxy-3-(methylamino)-N-[4-(trifluoromethyl)phenyl]chromane-8-sulfonamide;
(3R)—N-(3,4-Dichlorophenyl)-5-methoxy-3-(methylamino)chromane-8-sulfonamide;
(3R)-5-Methoxy-3-(methylamino)-N-[3-(trifluoromethyl)phenyl]chromane-8-sulfonamide;
(3R)-5-Methoxy-3-(methylamino)-N-quinolin-2-ylchromane-8-sulfonamide;
(3R)—N-(3-Cyanophenyl)-5-methoxy-3-(methylamino)chromane-8-sulfonamide;
(3R)—N-(4-Cyanophenyl)-5-methoxy-3-(methylamino)chromane-8-sulfonamide;
(3R)—N-(4-Chlorophenyl)-3-(dimethylamino)-5-methoxychromane-8-sulfonamide;
(3R)—N-(3-Cyanophenyl)-3-(dimethylamino)-5-methoxychromane-8-sulfonamide; and
(3R)—N-(4-Cyanophenyl)-3-(dimethylamino)-5-methoxychromane-8-sulfonamide;
or salts, solvates, or solvated salts thereof.

11-13. (canceled)

14. A pharmaceutical composition comprising as active ingredient a therapeutically effective amount of a compound according to claim 1, in association with one or more pharmaceutically acceptable diluents, excipients, and/or inert carriers.

15. A method of treating a 5HT6 mediated disorder, Alzheimer's disease, cognitive impairment associated with schizophrenia, obesity, and/or Parkinson's disease comprising administering a pharmaceutical composition according to claim 14.

16. A method of treating a 5HT6 mediated disorder, Alzheimer's disease, cognitive impairment associated with schizophrenia, obesity, and/or Parkinson's disease comprising administering to a mammal in need of such treatments a therapeutically effective amount of a compound according to claim 1.

17. An agent for the prevention or treatment of Alzheimer's disease, cognitive impairment associated with schizophrenia, obesity, and/or Parkinson's disease comprising as active ingredient a compound according to claim 1.

18. A compound selected from
(6S)-6-(Dimethylamino)-4-methoxy-5,6,7,8-tetrahydronaphthalene-1-sulfonyl chloride,
(6R)-6-(dimethylamino)-4-methoxy-5,6,7,8-tetrahydronaphthalene-1-sulfonyl chloride,
(6S)-4-methoxy-6-[(trifluoroacetyl)amino]-5,6,7,8-tetrahydronaphthalene-s-sulfonyl chloride,
(3R)-5-methoxy-3-[(trifluoroacetyl)amino]chromane-8-sulfonyl chloride,
(3R)-5-ethyl-3-[(trifluoroacetyl)amino]chromane-8-sulfonyl chloride, and
(3R)-6-chloro-3-[(trifluoroacetyl)amino]chromane-8-sulfonyl chloride.

19. (canceled)

20. A method of treatment according to claim 16, wherein said mammal is man.

21. A pharmaceutical composition comprising as active ingredient a therapeutically effective amount of a compound according to claim 9, in association with one or more pharmaceutically acceptable diluents, excipients and/or inert carriers.

22. A pharmaceutical composition comprising as active ingredient a therapeutically effective amount of the compound according to claim 10, in association with one or more pharmaceutically acceptable diluents, excipients and/or inert carriers.

23. A method of treating a 5HT6 mediated disorder, Alzheimer's disease, cognitive impairment associated with schizophrenia, obesity and/or Parkinson's disease comprising administering to a mammal in need of such treatment a therapeutically effective amount of a compound according to claim 9.

24. A method of treating a 5HT6 mediated disorder, Alzheimer's disease, cognitive impairment associated with schizophrenia, obesity and/or Parkinson's disease comprising administering to a mammal in need of such treatment a therapeutically effective amount of a compound according to claim 10.
